(19) United States
(12) Patent Application Publication
(10) Pub. No.: US 2026/0299410 A1
(43) Pub. Date: Oct. 1, 2026

(54) RESIST COMPOSITION AND RESIST PATTERN FORMATION METHOD

(71) Applicant: TOKYO OHKA KOGYO CO., LTD., Kawasaki-shi (JP)

(72) Inventors: Mari Murata, Kawasaki-shi (JP); Tatsuya Fujii, Kawasaki-shi (JP); Yushi Inaba, Kawasaki-shi (JP)

(21) Appl. No.: 19/489,871

(22) PCT Filed: Jun. 6, 2024
(86) PCT No.: PCT/JP2024/020750

§ 371 Date: Dec. 3, 2025

(30) Foreign Application Priority Data
Jun. 6, 2023 (JP) 2023-093352

(51) Int. Cl. G03F 7/004; C08L 25/18; C08L 33/10; G03F 7/038; G03F 7/039; G03F 7/32; H10P 76/20

(52) U.S. Cl. CPC G03F 7/0046; C08L 25/18; C08L 33/10; G03F 7/0045; G03F 7/0382; G03F 7/0388; G03F 7/0395; G03F 7/322; H10P 76/2041

(57) ABSTRACT

A resist composition including a base material component, an acid generator component containing a compound represented by Formula (b0), and a fluorine additive component having a structural unit represented by Formula (f0). In the formulas, Rpg is an acid-decomposable group; R1⁰¹ and R1⁰² are a cyclic hydrocarbon group; L⁰¹ is a divalent linking group or a single bond; L⁰² and L⁰³ are a divalent linking group; R^{m1} is a substituent other than an iodine atom; Vb⁰ is an alkylene group, a fluorinated alkylene group or the like; R⁰ is a fluorinated alkyl group, a fluorine atom or the like; nb1 is an integer of 1 to 4; nb2 is 1 to 4; nb3 is an integer of 0 to 3; mb1 and mb2 are 0 or 1; M^{m+} is an m-valent organic cation; R is an alkyl group, a halogenated alkyl group or the like; X⁰ is a divalent linking group having no acid-dissociable moiety; and Rf⁰ is an organic group having a fluorine atom

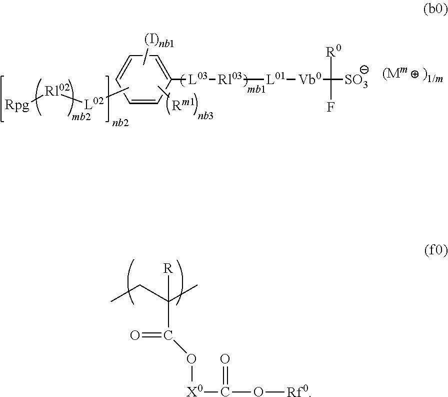

### TECHNICAL FIELD

The present invention relates to a resist composition, a resist pattern formation method, a compound and a polymer compound.

Priority is claimed on Japanese Patent Application No. 2023-093352, filed Jun. 6, 2023, the content of which is incorporated herein by reference.

### BACKGROUND ART

In recent years, in the production of semiconductor elements and liquid crystal display elements, advances in lithography technology have led to rapid progress in refinement of patterns. In refinement methods, exposure light sources with shorter wavelengths (higher energy) are generally used.

Resist materials are required to have lithography properties such as sensitivity to these exposure light sources and a resolution at which fine pattern dimensions can be reproduced.

As a resist material that satisfies such requirements, a chemically amplified resist composition that contains a base material component whose solubility in a development solution changes under the action of an acid and an acid generator component that generates an acid upon exposure has been conventionally used.

In addition, in order to improve lithography properties of the resist composition, it has been proposed to add a fluorine additive component that imparts water repellency to a resist film.

For example, Patent Document 1 describes a resist composition including a base material component whose solubility in a development solution changes under the action of an acid, an acid generator component that generates an acid upon exposure, and a fluorine additive component.

### CITATION LIST

### Patent Document

Patent Document 1: Japanese Unexamined Patent Application, First Publication No. 2018-022039

### SUMMARY OF INVENTION

### Technical Problem

As lithography technology continues to advance and resist patterns become increasingly finer, for example, in EUV or EB lithography, the object is to form fine patterns of several tens of nm. As resist pattern dimensions become smaller in this manner, it is required to further improve lithography properties such as sensitivity, resolution and CDU.

The present invention has been made in view of the above circumstances, and an object of the present invention is to provide a resist composition that exhibits favorable sensitivity, resolution, and CDU in all respects and a resist pattern formation method using the resist composition.

### Solution to Problem

In order to achieve the above object, the present invention provides the following aspects.

That is, a first aspect of the present invention is a resist composition that generates an acid upon exposure and whose solubility in a development solution changes under the action of the acid, including a base material component (A) whose solubility in a development solution changes under the action of an acid, an acid generator component (B) that generates an acid upon exposure, and a fluorine additive component (F) that is decomposable in an alkaline development solution, wherein the acid generator component (B) contains a compound (B0) represented by the following General Formula (b0), and the fluorine additive component (F) contains a fluorine resin component (F0) having a structural unit (f0) represented by the following General Formula (f0).

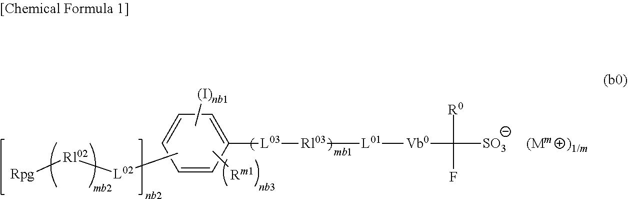

[in the formula, Rpg is an acid-decomposable group, R1⁰¹ and R1⁰² are each independently a cyclic hydrocarbon group which may have a substituent, L⁰¹ is a divalent linking group or a single bond, L⁰² and L⁰³ are each independently a divalent linking group, R^{m1} is a substituent other than an iodine atom, Vb⁰ is a single bond, an alkylene group or a fluorinated alkylene group, R⁰ is a hydrogen atom, a fluorinated alkyl group having 1 to 5 carbon atoms or a fluorine atom, nb1 is an integer of 1 to 4, nb2 is an integer of 1 to 4, nb3 is an integer of 0 to 3, and nb1+nb2+nb3≤5, mb1 and mb2 are each independently 0 or 1, and nb1 and mb2 are not both 0, M^{m+} is an m-valent organic cation, and m is an integer of 1 or more].

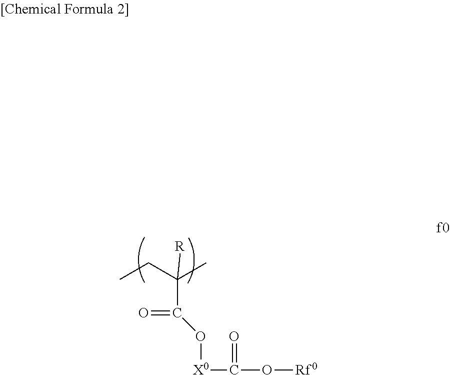

[in the formula, R is a hydrogen atom, an alkyl group having 1 to 5 carbon atoms or a halogenated alkyl group having 1 to 5 carbon atoms, X⁰ is a divalent linking group having no acid-dissociable moiety, and Rf⁰ is an organic group having a fluorine atom].

A second aspect of the present invention is a resist pattern formation method including a step of forming a resist film on a support using the resist composition according to the first aspect, a step of exposing the resist film, and a step of developing the exposed resist film to form a resist pattern.

### Advantageous Effects of Invention

According to the present invention, it is possible to provide a resist composition that exhibits favorable sensitivity, resolution, and CDU in all respects, a resist pattern formation method using the resist composition, a polymer compound that can be used to produce the resist composition, and a compound that can be used to synthesize the polymer compound.

### DESCRIPTION OF EMBODIMENTS

In the scope of this specification and claims, the term “aliphatic” is a relative concept with respect to the term “aromatic,” and defines a group, compound or the like which has no aromaticity.

Unless otherwise specified, the term “alkyl group” includes linear, branched and cyclic monovalent saturated hydrocarbon groups. The same applies to alkyl groups in alkoxy groups.

Unless otherwise specified, the term “alkylene group” includes linear, branched and cyclic divalent saturated hydrocarbon groups.

The term “halogen atom” includes a fluorine atom, a chlorine atom, a bromine atom, an iodine atom, and the like.

The term “structural unit” refers to a monomer unit (monomeric unit) constituting a polymer compound (a resin, a polymer, or a copolymer).

The description “may have a substituent” includes both a case in which a hydrogen atom (—H) is substituted with a monovalent group and a case in which a methylene group (—CH₂—) is substituted with a divalent group.

The term “exposure” is a concept that includes irradiation with any form of radiation.

The term “acid-decomposable group” refers to a group having acid-decomposability in which at least some bonds in the structure of the acid-decomposable group can be cleaved under the action of an acid.

Examples of acid-decomposable groups whose polarity increases under the action of an acid include groups that decompose under the action of an acid to generate a polar group.

Examples of polar groups include a carboxy group, a hydroxy group, an amino group, and a sulfo group (—SO₃H).

More specific examples of acid-decomposable groups include groups in which a polar group is protected with an acid-dissociable group (for example, a group in which a hydrogen atom of an OH-containing polar group is protected with an acid-dissociable group).

The term “acid-dissociable group” refers to both (i) a group having acid-dissociability in which the bond between the acid-dissociable group and an atom adjacent to the acid-dissociable group can be cleaved under the action of an acid and (ii) a group in which some bonds are cleaved under the action of an acid, then a decarboxylation reaction additionally occurs, and thus the bond between the acid-dissociable group and an atom adjacent to the acid-dissociable group can be cleaved.

The acid-dissociable group constituting the acid-decomposable group needs to be a group having a lower polarity than the polar group generated by dissociation of the acid-dissociable group, and accordingly, when the acid-dissociable group is dissociated under the action of an acid, a polar group having a higher polarity than the acid-dissociable group is generated and the polarity increases. As a result, the polarity of the entire component (A1) increases. When the polarity increases, the relative solubility in the development solution changes, when the development solution is an alkaline development solution, the solubility increases, and when the development solution is an organic development solution, the solubility decreases.

The term “base material component” is an organic compound having a film-forming ability. Organic compounds used as the base material component are broadly classified into non-polymers and polymers. Generally, regarding the non-polymer, one having a molecular weight of 500 or more and less than 4,000 is used (hereinafter referred to as a “low-molecular-weight compound”). Hereinafter, the “resin,” “polymer compound” or “polymer” refers to a polymer having a molecular weight of 1,000 or more. Regarding the molecular weight of the polymer, the weight average molecular weight in terms of polystyrene determined through gel permeation chromatography (GPC) is used.

The term “derived structural unit” refers to a structural unit formed by cleavage of multiple bonds between carbon atoms, for example, an ethylenic double bond.

In the “acrylic acid ester,” a hydrogen atom bonded to the carbon atom at the α-position may be substituted with a substituent. The substituent (R^{αx}) that substitutes a hydrogen atom bonded to the carbon atom at the α-position is an atom or group other than a hydrogen atom. In addition, it also includes an itaconic acid diester in which the substituent (R^{αx}) is substituted with a substituent containing an ester bond and an α-hydroxyacrylic ester in which the substituent (R^{αx}) is substituted with a hydroxyalkyl group or a group modified with a hydroxy group. Here, unless otherwise specified, the carbon atom at the α-position of the acrylic acid ester is a carbon atom to which a carbonyl group of acrylic acid is bonded.

Hereinafter, an acrylic acid ester in which a hydrogen atom bonded to the carbon atom at the α-position is substituted with a substituent will sometimes be referred to as an α-substituted acrylic acid ester.

The term “derivative” is a concept including a compound in which the hydrogen atom at the α-position of a target compound is substituted with another substituent such as an alkyl group or a halogenated alkyl group and derivatives thereof. Examples of such derivatives include those in which a hydrogen atom of a hydroxy group of a target compound in which the hydrogen atom at the α-position may be substituted with a substituent is substituted with an organic group; and those in which a substituent other than a hydroxy group is bonded to a target compound in which the hydrogen atom at the α-position may be substituted with a substituent. Here, unless otherwise specified, the carbon atom at the α-position refers to a first carbon atom adjacent to a functional group.

Examples of substituents that substitute the hydrogen atom at the α-position of hydroxystyrene include the same substituents as for R^{αx}.

In the scope of this specification and claims, some structures represented by chemical formulae may have asymmetric carbon atoms, and may have enantiomers or diastereomers. In such cases, one chemical formula represents the isomers. These isomers may be used alone or as a mixture.

### (Resist Composition)

The resist composition of the present embodiment generates an acid upon exposure and whose solubility in a development solution changes under the action of the acid.

The resist composition contains a base material component (A) whose solubility in a development solution changes under the action of an acid (hereinafter referred to as a “component (A)”), an acid generator component (B) that generates an acid upon exposure (hereinafter referred to as a “component (B)”), and a fluorine additive component (F) that is decomposable in an alkaline development solution (hereinafter referred to as a “component (F)”).

When a resist film is formed using the resist composition of the present embodiment and the resist film is subjected to selective exposure, in an exposed part of the resist film, an acid is generated from the component (B), and the solubility of the component (A) in a development solution changes under the action of the acid, but in an unexposed part of the resist film, the solubility of the component (A) in a development solution does not change. Therefore, a difference in solubility in a development solution occurs between the exposed part and the unexposed part. Therefore, when the resist film is developed, if the resist composition is of a positive type, the exposed part of the resist film is dissolved and removed to form a positive type resist pattern, and if the resist composition is of a negative type, the unexposed part of the resist film is dissolved and removed to form a negative type resist pattern.

The resist composition of the present embodiment may be a positive type resist composition or a negative type resist composition. In addition, the resist composition of the present embodiment may be used for an alkaline development process in which an alkaline development solution is used in a development treatment during resist pattern formation or for a solvent development process in which a development solution containing an organic solvent (organic development solution) is used in the development treatment.

### <Base Material Component (A)>

In the resist composition of the present embodiment, the component (A) containing a resin component (A1) whose solubility in a development solution changes under the action of the acid (hereinafter referred to as a “component (A1)”) is preferably used. When the component (A1) is used, since the polarity of the base material component changes between before and after exposure, a favorable development contrast can be obtained not only in the alkaline development process but also in the solvent development process.

As the component (A), other polymer compounds and/or low-molecular-weight compounds may be used in combination with the component (A1).

In the resist composition of the present embodiment, the components (A) may be used alone or two or more thereof may be used in combination.

### Component (A1)

The component (A1) is a resin component whose solubility in a development solution changes under the action of an acid.

The component (A1) preferably has a structural unit (a1) having an acid-decomposable group whose polarity increases under the action of an acid.

The component (A1) may have, as necessary, other structural units, in addition to the structural unit (a1).

### <<Structural Unit (A1)>>

The structural unit (a1) is a structural unit having an acid-decomposable group whose polarity increases under the action of an acid.

Examples of acid-dissociable groups include those that have been previously proposed as acid-dissociable groups in base resins for chemically amplified resist compositions.

Specific examples of those proposed as acid-dissociable groups in base resins for chemically amplified resist compositions include the following “acetal-type acid-dissociable group,” “tertiary alkyl ester type acid-dissociable group,” “tertiary alkyloxycarbonyl acid-dissociable group,” and “secondary alkyloxycarbonyl acid-dissociable group.”

### Acetal-Type Acid-Dissociable Group:

Examples of acid-dissociable groups that protect a carboxy group or a hydroxy group among polar groups include an acid-dissociable group represented by the following General Formula (a1-r-1) (hereinafter sometimes referred to as an “acetal-type acid-dissociable group”).

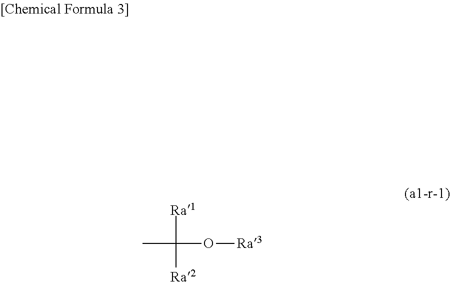

[in the formula, Ra′¹ and Ra′² are a hydrogen atom or an alkyl group, Ra′³ is a hydrocarbon group, and Ra′³ may be bonded to either Ra′¹ or Ra′² to form a ring].

In Formula (a1-r-1), it is preferable that at least one of Ra′¹ and Ra′² be a hydrogen atom, and it is more preferable that both of Ra′¹ and Ra′² be a hydrogen atom.

When Ra′¹ or Ra′² is an alkyl group, as the alkyl group, the same alkyl groups exemplified as the substituent that may be bonded to the carbon atom at the α-position in the description of the α-substituted acrylic acid ester may be exemplified, and an alkyl group having 1 to 5 carbon atoms is preferable. Specifically, linear or branched alkyl groups are preferably exemplified. More specific examples thereof include a methyl group, an ethyl group, a propyl group, an isopropyl group, an n-butyl group, an isobutyl group, a tert-butyl group, a pentyl group, an isopentyl group, and a neopentyl group. A methyl group or an ethyl group is more preferable, and a methyl group is particularly preferable.

In Formula (a1-r-1), examples of hydrocarbon groups for Ra′³ include a linear or branched alkyl group and a cyclic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among these, a methyl group, an ethyl group or an n-butyl group is preferable, and a methyl group or an ethyl group is more preferable.

The branched alkyl group preferably has 3 to 10 carbon atoms and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group, and an isopropyl group is preferable.

When Ra′³ is a cyclic hydrocarbon group, the hydrocarbon group may be an aliphatic hydrocarbon group or an aromatic hydrocarbon group, and may be a polycyclic group or a monocyclic group.

The aliphatic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom has been removed from a polycycloalkane, the polycycloalkane is preferably one having 7 to 12 carbon atoms, and specific examples thereof include adamantane, norbornane, isobornane, tricyclo[5.2.1.0^{2,6}]decane, and tetracyclododecane.

When the cyclic hydrocarbon group for Ra′³ is an aromatic hydrocarbon group, the aromatic hydrocarbon group is a hydrocarbon group having at least one aromatic ring.

The aromatic ring is not particularly limited as long as it is a cyclic conjugated system having 4n+2 π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms.

Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

Specific examples of aromatic hydrocarbon groups for Ra′³ include a group in which one hydrogen atom has been removed from the aromatic hydrocarbon ring or aromatic heterocycle (an aryl group or a heteroaryl group); a group in which one hydrogen atom has been removed from an aromatic compound containing two or more aromatic rings (for example, biphenyl, fluorine, etc.); and a group in which one hydrogen atom of the aromatic hydrocarbon ring or aromatic heterocycle is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group). The alkylene group bonded to the aromatic hydrocarbon ring or aromatic heterocycle preferably has 1 to 4 carbon atoms, more preferably has 1 or 2 carbon atoms, and particularly preferably has 1 carbon atom.

The cyclic hydrocarbon group for Ra′³ may have a substituent. Examples of substituents include —R^{P1}, —R^{P2}—O—R^{P1}, —R^{P2}—CO—R^{P1}, —R^{P2}—CO—OR^{P1}, —R^{P2}—O—CO-RPi, —R^{P2}—OH, —R^{P2}—CN and —R^{P2}—COOH (hereinafter these substituents are collectively referred to as “Ra^{x5}”).

Here, R^{P1} is a monovalent chain saturated hydrocarbon group having 1 to 10 carbon atoms, a monovalent aliphatic cyclic saturated hydrocarbon group having 3 to 20 carbon atoms or a monovalent aromatic hydrocarbon group having 6 to 30 carbon atoms. In addition, R^{P2} is a single bond, a divalent chain saturated hydrocarbon group having 1 to 10 carbon atoms, a divalent aliphatic cyclic saturated hydrocarbon group having 3 to 20 carbon atoms or a divalent aromatic hydrocarbon group having 6 to 30 carbon atoms. Here, some or all of the hydrogen atoms in the chain saturated hydrocarbon group, the aliphatic cyclic saturated hydrocarbon group and the aromatic hydrocarbon group for R^{P}, and R^{P2} may be substituted with fluorine atoms. The aliphatic cyclic hydrocarbon group may have one or more of one type of the substituents or may have one or more of each type of a plurality of substituents.

Examples of monovalent chain saturated hydrocarbon groups having 1 to 10 carbon atoms include a methyl group, an ethyl group, a propyl group, a butyl group, a pentyl group, a hexyl group, a heptyl group, an octyl group, and a decyl group.

Examples of monovalent aliphatic cyclic saturated hydrocarbon groups having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.0^{2,6}]decanyl group, a tricyclo[3.3.1.1^{3,7}]decanyl group, a tetracyclo[6.2.1.1^{3,6}.0^{2,7}]dodecanyl group, and an adamantyl group.

Examples of monovalent aromatic hydrocarbon groups having 6 to 30 carbon atoms include groups in which one hydrogen atom has been removed from an aromatic hydrocarbon ring such as benzene, biphenyl, fluorene, naphthalene, anthracene, and phenanthrene.

When Ra′³ is bonded to either Ra′¹ or Ra′² to form a ring, the cyclic group is preferably a 4- to 7-membered ring and more preferably a 4- to 6-membered ring. Specific examples of cyclic groups include a tetrahydropyranyl group and a tetrahydrofuranyl group.

### Tertiary Alkyl Ester Type Acid-Dissociable Group:

Examples of acid-dissociable groups that protect a carboxy group among the above polar groups include an acid-dissociable group represented by the following General Formula (a1-r-2).

Here, among the acid-dissociable groups represented by the following Formula (a1-r-2), one constituted by an alkyl group will sometimes be referred to hereinafter as a “tertiary alkyl ester type acid-dissociable group,” for convenience.

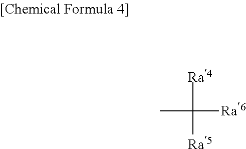

[in the formula, Ra′⁴ to Ra′⁶ are each a hydrocarbon group, and Ra′⁵ and Ra′⁶ may be bonded to each other to form a ring].

Examples of hydrocarbon groups for Ra′⁴ include a linear or branched alkyl group, a chain or cyclic alkenyl group, and a cyclic hydrocarbon group.

Examples of linear or branched alkyl groups and cyclic hydrocarbon groups (an aliphatic hydrocarbon group which is a monocyclic group, an aliphatic hydrocarbon group which is a polycyclic group, and an aromatic hydrocarbon group) for Ra′⁴ include the same groups as for Ra′³.

The chain or cyclic alkenyl group for Ra′⁴ is preferably an alkenyl group having 2 to 10 carbon atoms.

Examples of hydrocarbon groups for Ra′⁵ and Ra′⁶ include the same groups as for Ra′³.

When Ra′⁵ and Ra′⁶ are bonded to each other to form a ring, preferable examples thereof include a group represented by the following General Formula (a1-r2-1), a group represented by the following General Formula (a1-r2-2), and a group represented by the following General Formula (a1-r2-3).

On the other hand, when Ra′⁴ to Ra′⁶ are not bonded to each other and are independently a hydrocarbon group, preferable examples thereof include a group represented by the following General Formula (a1-r2-4).

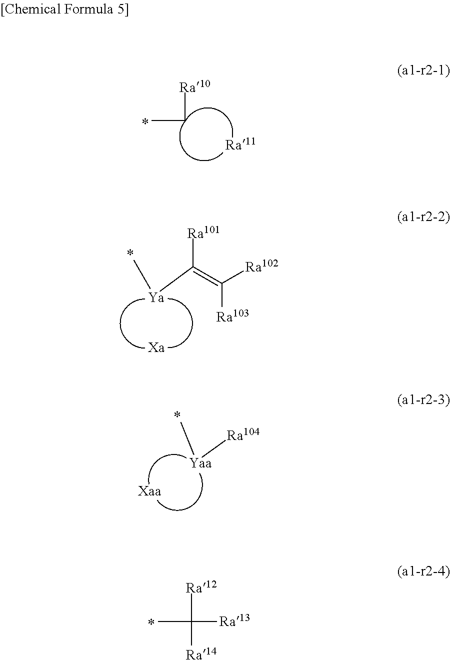

[in Formula (a1-r2-1), Ra′¹⁰ is a linear or branched alkyl group having 1 to 12 carbon atoms, some of which may be substituted with a halogen atom or a heteroatom-containing group, Ra′¹¹ is a group that forms an aliphatic cyclic group together with a carbon atom to which Ra′¹⁰ is bonded, in Formula (a1-r2-2), Ya is a carbon atom, Xa is a group which forms a cyclic hydrocarbon group together with Ya, some carbon atoms constituting the ring of the cyclic hydrocarbon group may be substituted with a heteroatom-containing group, Ra¹⁰¹ to Ra¹⁰³ are each independently a hydrogen atom, a monovalent chain saturated hydrocarbon group having 1 to 10 carbon atoms or a monovalent aliphatic cyclic saturated hydrocarbon group having 3 to 20 carbon atoms, some or all of the hydrogen atoms in the chain saturated hydrocarbon group and the aliphatic cyclic saturated hydrocarbon group may be substituted, two or more of Ra¹⁰¹ to Ra¹⁰³ may be bonded to each other to form a ring structure, in Formula (a1-r2-3), Yaa is a carbon atom, Xaa is a group which forms an aliphatic cyclic group together with Yaa, Ra¹⁰⁴ is an aromatic hydrocarbon group which may have a substituent, in Formula (a1-r2-4), Ra′¹² and Ra′¹³ are each independently a monovalent chain saturated hydrocarbon group having 1 to 10 carbon atoms, some or all of the hydrogen atoms in the chain saturated hydrocarbon group may be substituted, Ra′¹⁴ is a hydrocarbon group which may have a substituent, and * indicates a bond (the same applies hereinafter)].

In Formula (a1-r2-1), Ra′¹⁰ is a linear or branched alkyl group having 1 to 12 carbon atoms, some of which may be substituted with a halogen atom or a heteroatom-containing group.

The linear alkyl group for Ra′¹⁰ has 1 to 12 carbon atoms, preferably has 1 to 10 carbon atoms, and particularly preferably has 1 to 5 carbon atoms.

Examples of branched alkyl groups for Ra′¹⁰ include the same groups as for Ra′³.

Some carbon atoms in the alkyl group for Ra′¹⁰ may be substituted with a halogen atom or a heteroatom-containing group. For example, some hydrogen atoms constituting the alkyl group may be substituted with a halogen atom or a heteroatom-containing group. In addition, some carbon atoms (a methylene group, etc.) constituting the alkyl group may be substituted with a heteroatom-containing group.

Examples of heteroatoms referred to here include an oxygen atom, a sulfur atom, and a nitrogen atom. Examples of heteroatom-containing groups include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In Formula (a1-r2-1), Ra′¹¹ (an aliphatic cyclic group formed together with a carbon atom to which Ra′¹⁰ is bonded) is preferably a group exemplified as an aliphatic hydrocarbon group (alicyclic hydrocarbon group) which is a monocyclic group or a polycyclic group for Ra′³ in Formula (a1-r-1). Among these, a monocyclic alicyclic hydrocarbon group is preferable, and specifically, a cyclopentyl group or a cyclohexyl group is more preferable.

In Formula (a1-r2-2), examples of cyclic hydrocarbon groups formed by Xa together with Ya include groups in which one or more hydrogen atoms have been additionally removed from a cyclic monovalent hydrocarbon group (aliphatic hydrocarbon group) for Ra′³ in Formula (a1-r-1).

The cyclic hydrocarbon group formed by Xa together with Ya may have a substituent. Examples of substituents include the same substituents that the cyclic hydrocarbon group for Ra′³ may have.

In Formula (a1-r2-2), examples of monovalent chain saturated hydrocarbon groups having 1 to 10 carbon atoms for Ra¹⁰¹ to Ra¹⁰³ include a methyl group, an ethyl group, a propyl group, a butyl group, a pentyl group, a hexyl group, a heptyl group, an octyl group, and a decyl group.

Examples of monovalent aliphatic cyclic saturated hydrocarbon groups having 3 to 20 carbon atoms for Ra¹⁰¹ to Ra¹⁰³ include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.0^{2,6}]decanyl group, a tricyclo[3.3.1.1^{3,7}]decanyl group, a tetracyclo[6.2.1.1^{3,6}.0^{2,7}]dodecanyl group, and an adamantyl group.

Among these, in consideration of ease of synthesis, Ra¹⁰¹ to Ra¹⁰³ are preferably a hydrogen atom or a monovalent chain saturated hydrocarbon group having 1 to 10 carbon atoms, and among these, a hydrogen atom, a methyl group, and an ethyl group are more preferable, and a hydrogen atom is particularly preferable.

Examples of substituents that the chain saturated hydrocarbon groups represented by Ra¹⁰¹ to Ra¹⁰³ or the aliphatic cyclic saturated hydrocarbon group have include the same groups as for Ra^{x5}.

Examples of groups containing a carbon-carbon double bond generated when two or more of Ra¹⁰¹ to Ra¹⁰³ are bonded to each other to form a ring structure include a cyclopentenyl group, a cyclohexenyl group, a methylcyclopentenyl group, a methylcyclohexenyl group, a cyclopentylideneethenyl group, and a cyclohexylideneethenyl group. Among these, in consideration of ease of synthesis, a cyclopentenyl group, a cyclohexenyl group, or a cyclopentylideneethenyl group is preferable.

In Formula (a1-r2-3), the aliphatic cyclic group formed by Yaa together with Xaa is preferably a group exemplified as an aliphatic hydrocarbon group which is a monocyclic group or a polycyclic group for Ra′³ in Formula (a1-r-1).

In Formula (a1-r2-3), examples of aromatic hydrocarbon groups for Ra¹⁰⁴ include a group in which one or more hydrogen atoms have been removed from an aromatic hydrocarbon ring having 5 to 30 carbon atoms. Among these, Ra¹⁰⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from benzene or naphthalene, and most preferably a group in which one or more hydrogen atoms have been removed from benzene.

Examples of substituents that Ra¹⁰⁴ in Formula (a1-r2-3) may have include a methyl group, an ethyl group, a propyl group, a hydroxy group, a carboxy group, a halogen atom, an alkoxy group (a methoxy group, an ethoxy group, a propoxy group, a butoxy group, etc.), and an alkyloxycarbonyl group.

In Formula (a1-r2-4), Ra′¹² and Ra′¹³ are each independently a monovalent chain saturated hydrocarbon group having 1 to 10 carbon atoms. Examples of monovalent chain saturated hydrocarbon groups having 1 to 10 carbon atoms for Ra′¹² and Ra′¹³ include the same as the monovalent chain saturated hydrocarbon group having 1 to 10 carbon atoms for Ra¹⁰¹ to Ra¹⁰³. Some or all of the hydrogen atoms in the chain saturated hydrocarbon group may be substituted.

Among these, Ra′¹² and Ra′¹³ are preferably an alkyl group having 1 to 5 carbon atoms, more preferably an alkyl group having 1 to 5 carbon atoms, still more preferably a methyl group or an ethyl group, and particularly preferably a methyl group.

When the chain saturated hydrocarbon groups represented by Ra′¹² and Ra′¹³ are substituted, examples of substituents include the same groups as for Ra^{x5}.

In Formula (a1-r2-4), Ra′¹⁴ is a hydrocarbon group which may have a substituent. Examples of hydrocarbon groups for Ra′¹⁴ include a linear or branched alkyl group and a cyclic hydrocarbon group.

The linear alkyl group for Ra′¹⁴ is preferably 1 to 5 carbon atoms, more preferably 1 to 4 carbon atoms, and still more preferably 1 or 2 carbon atoms. Specific examples thereof include a methyl group, an ethyl group, an n-propyl group, an n-butyl group, and an n-pentyl group. Among these, a methyl group, an ethyl group or an n-butyl group is preferable, and a methyl group or an ethyl group is more preferable.

The branched alkyl group for Ra′¹⁴ is preferably 3 to 10 carbon atoms and more preferably 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group, and an isopropyl group is preferable.

When Ra′¹⁴ is a cyclic hydrocarbon group, the hydrocarbon group may be an aliphatic hydrocarbon group or an aromatic hydrocarbon group and may be a polycyclic group or a monocyclic group.

The aliphatic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom has been removed from a polycycloalkane, the polycycloalkane is preferably one having 7 to 12 carbon atoms, and specific examples thereof include adamantane, norbornane, isobornane, tricyclo[5.2.1.0^{2,6}]decane, and tetracyclododecane.

Examples of aromatic hydrocarbon groups for Ra′¹⁴ include the same aromatic hydrocarbon groups as for Ra¹⁰⁴. Among these, Ra′¹⁴ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from naphthalene or anthracene, and most preferably a group in which one or more hydrogen atoms have been removed from naphthalene.

Examples of substituents that Ra′¹⁴ may have include the same substituents that Ra¹⁰⁴ may have.

When Ra′¹⁴ in Formula (a1-r2-4) is a naphthyl group, the position at which it is bonded to the tertiary carbon atom in Formula (a1-r2-4) may be either the 1st or 2nd position of the naphthyl group.

When Ra′¹⁴ in Formula (a1-r2-4) is an anthryl group, the position at which it is bonded to the tertiary carbon atom in Formula (a1-r2-4) may be any of the 1st, 2nd, and 9th positions of the anthryl group.

Specific examples of groups represented by Formula (a1-r2-1) are shown below.

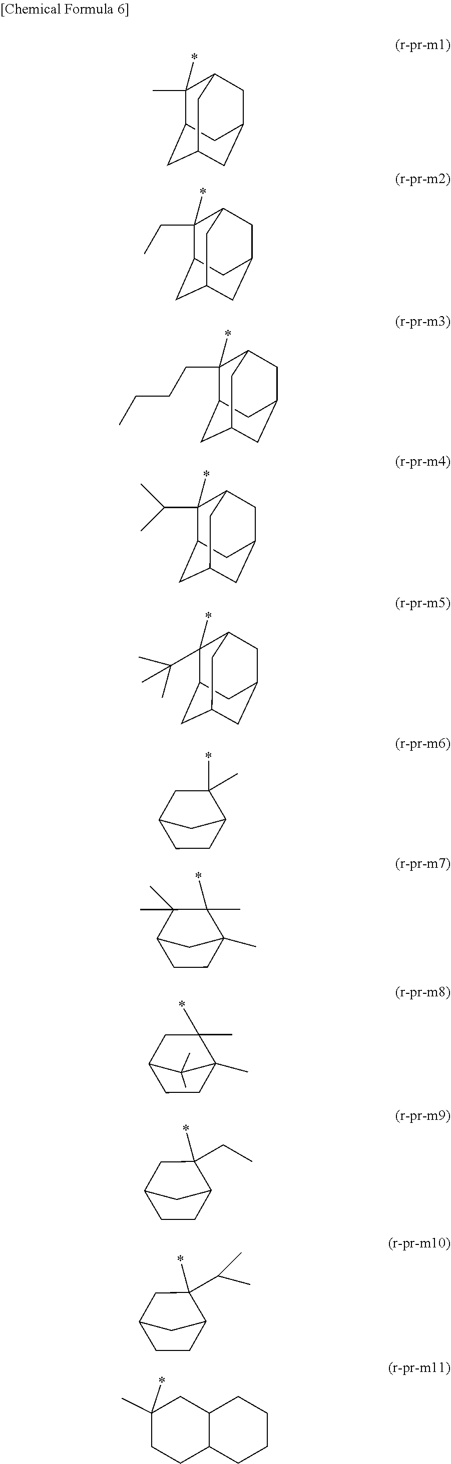

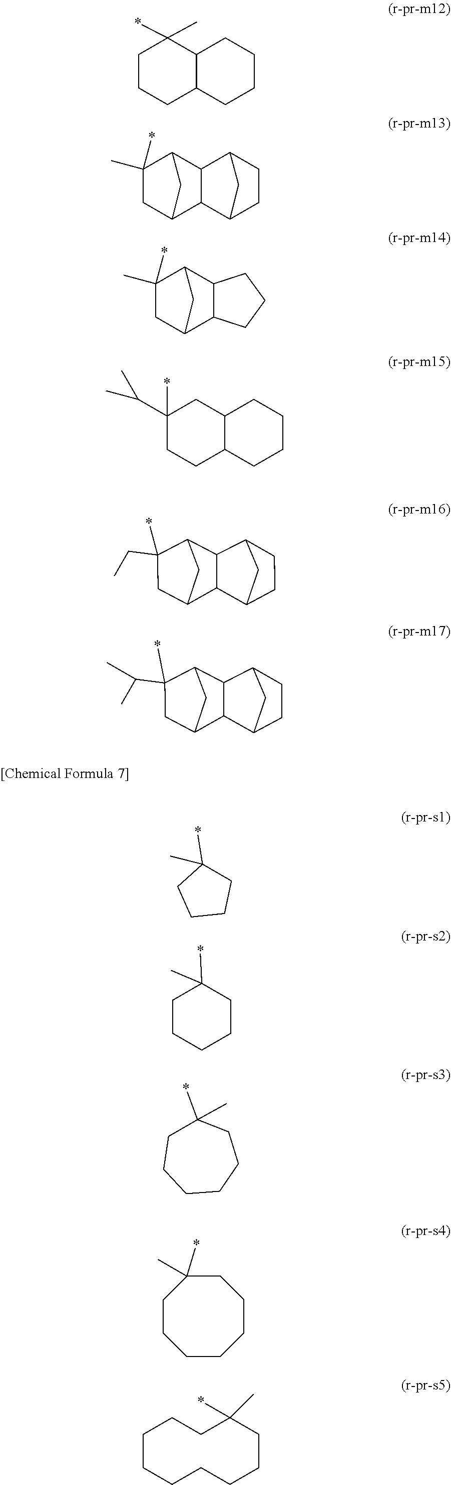

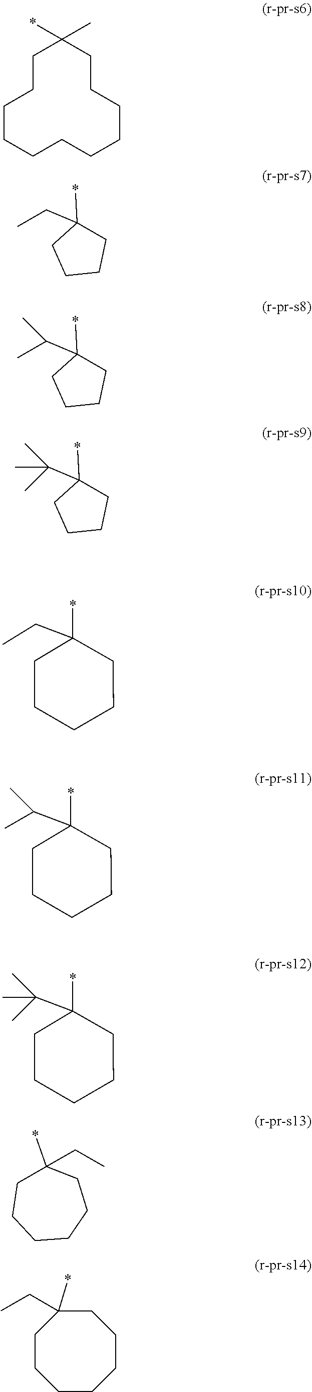

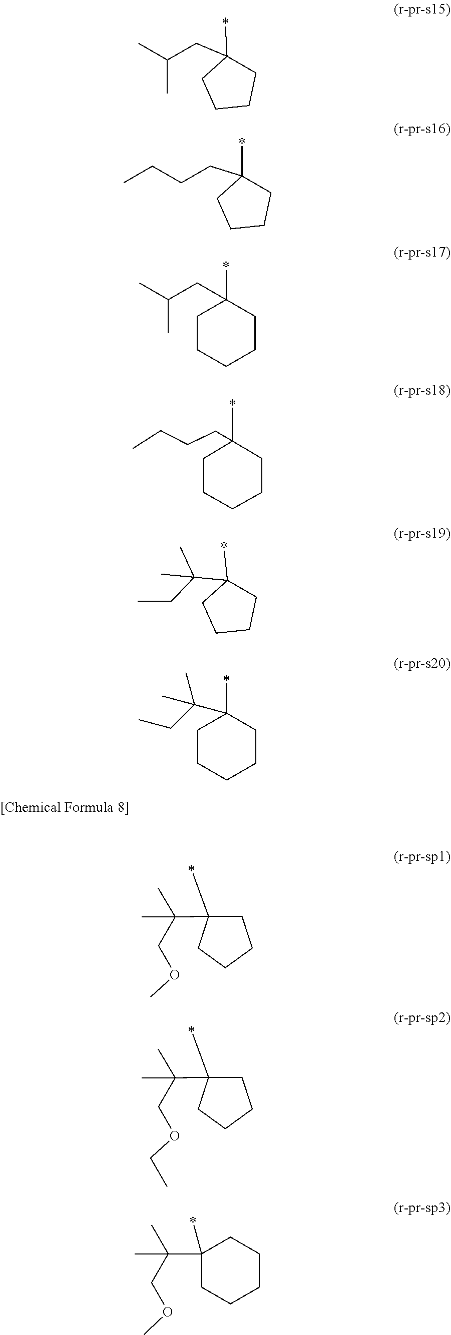

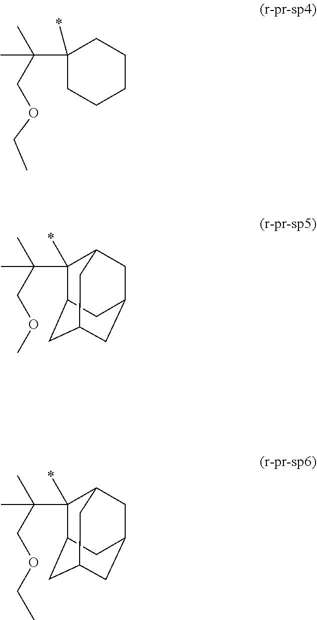 [SMILES: *C1(C(C)(C)COCC)CCCCC1 ; *C1(C(C)(C)COC)C2CC3CC(C2)CC1C3 ; *C1(C(C)(C)COCC)C2CC3CC(C2)CC1C3]

Specific examples of groups represented by Formula (a1-r2-2) are shown below.

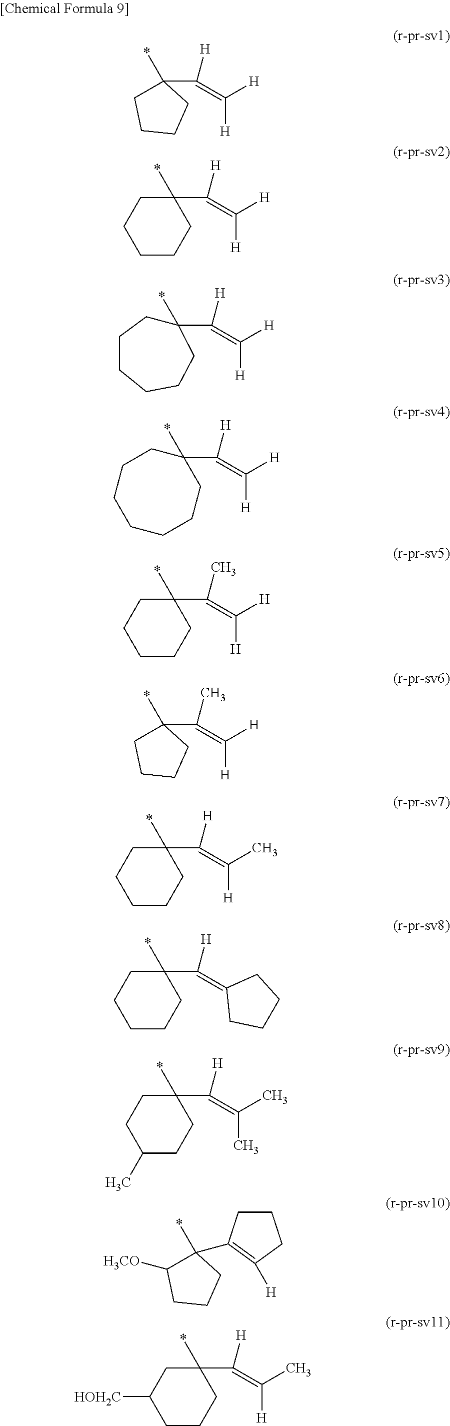

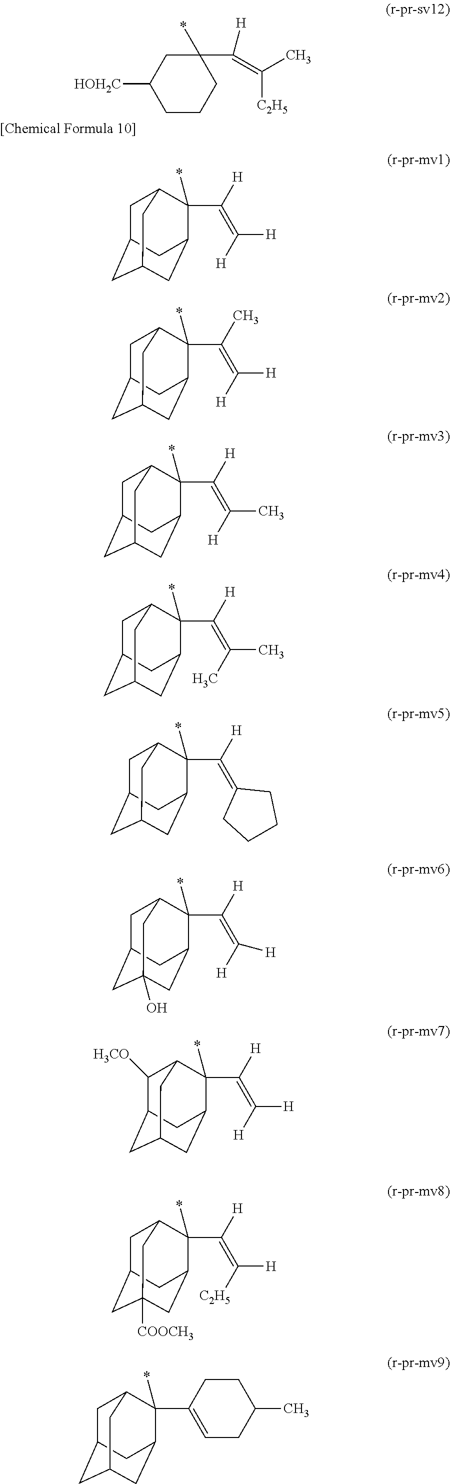

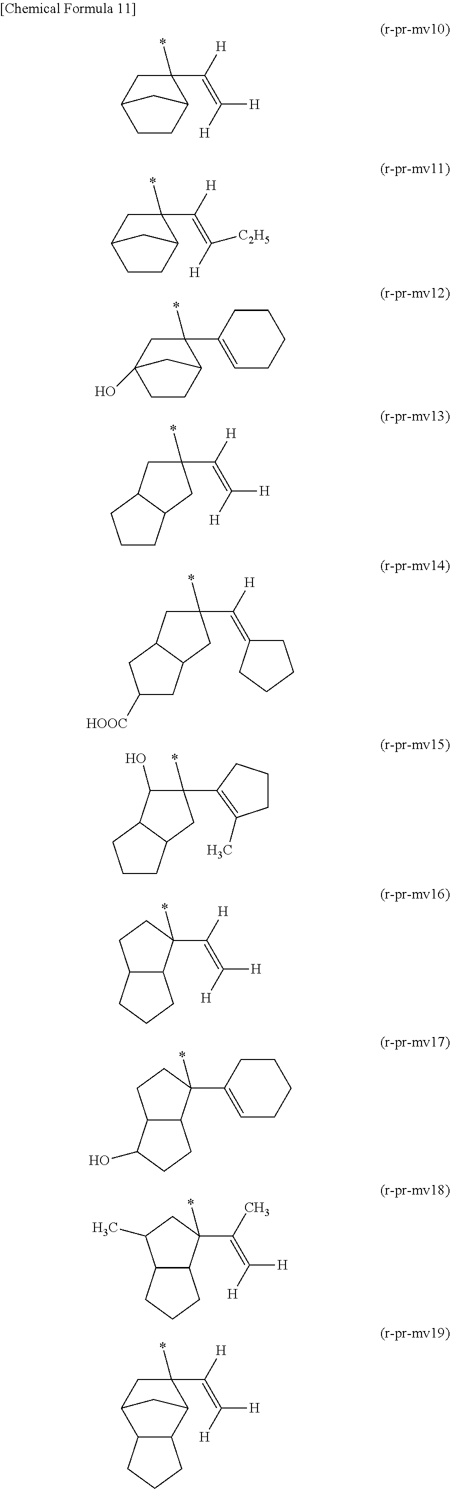

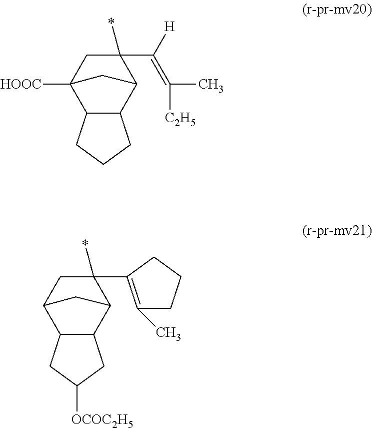

Specific examples of groups represented by Formula (a1-r2-3) are shown below.

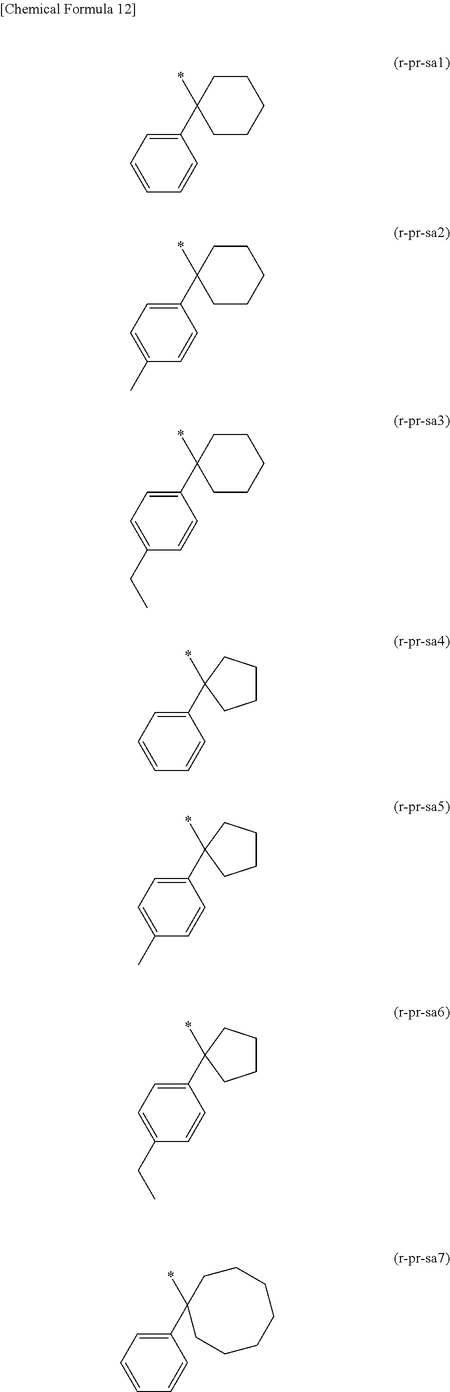

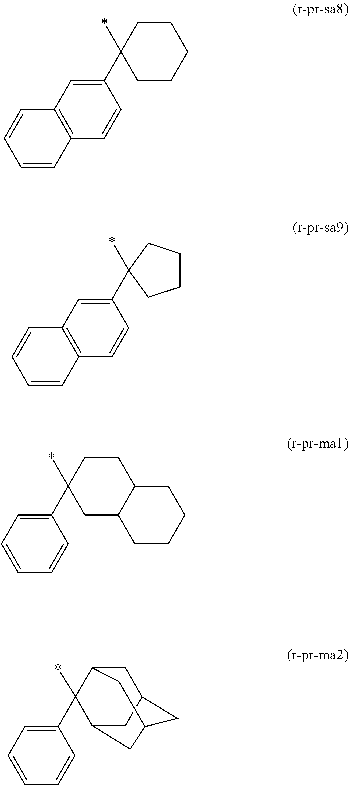 [SMILES: *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of groups represented by Formula (a1-r2-4) are shown below.

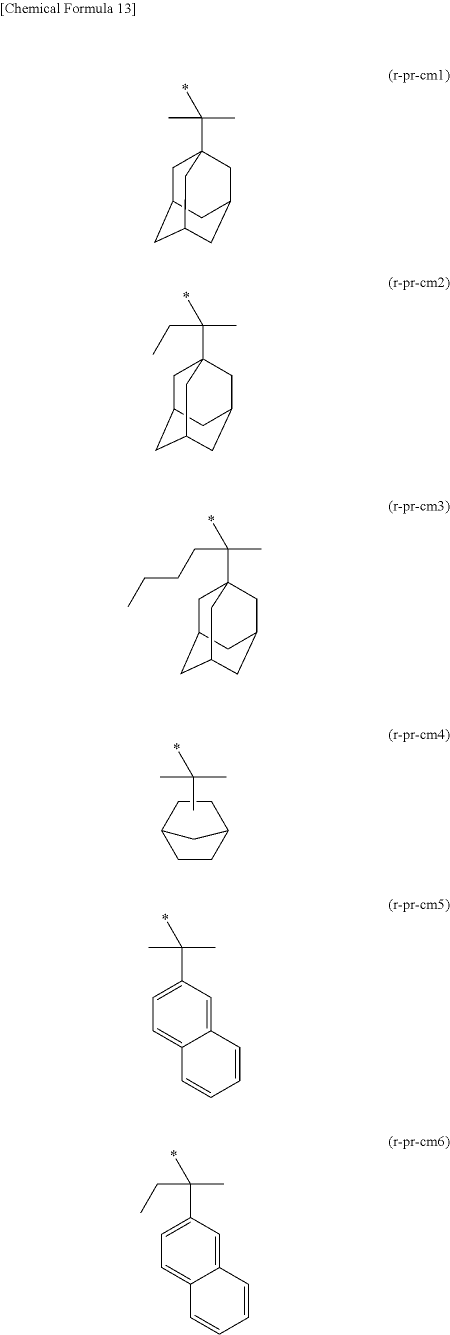

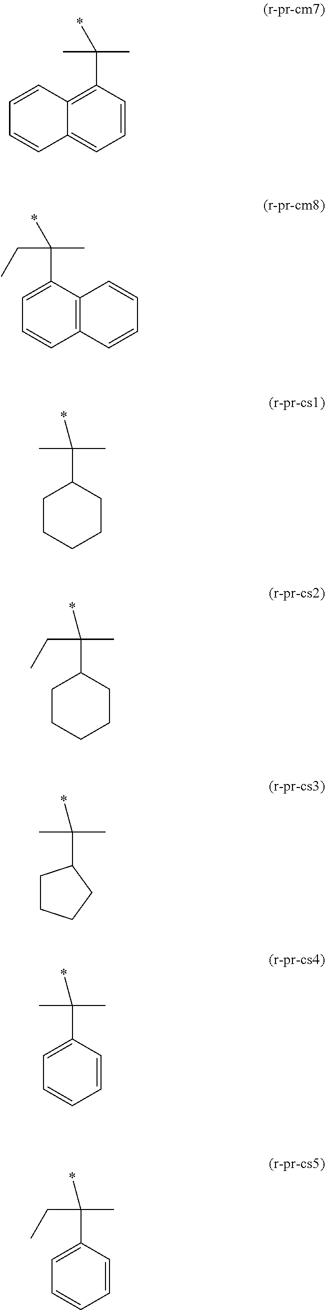

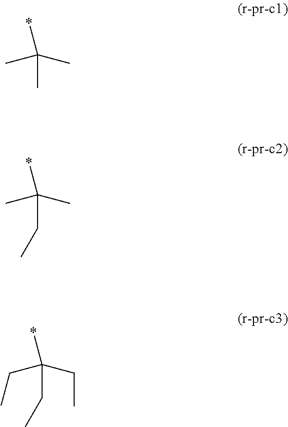

### Tertiary Alkyloxycarbonyl Acid-Dissociable Group:

Examples of acid-dissociable groups that protect a hydroxy group among the polar groups include an acid-dissociable group represented by the following General Formula (a1-r-3) (will sometimes be referred to hereinafter as a “tertiary alkyloxycarbonyl acid-dissociable group” for convenience).

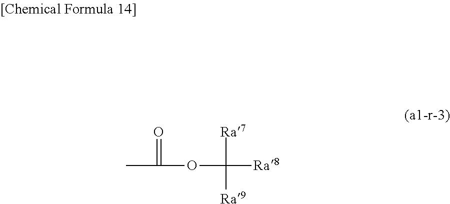

[in the formula, Ra′⁷ to Ra′⁹ are each an alkyl group].

In Formula (a1-r-3), Ra′⁷ to Ra′⁹ are each preferably an alkyl group having 1 to 5 carbon atoms, and more preferably an alkyl group having 1 to 3 carbon atoms.

In addition, each alkyl group preferably has a total number of carbon atoms of 3 to 7, more preferably has 3 to 5 carbon atoms, and most preferably has 3 and 4 carbon atoms.

### Secondary Alkyl Ester Type Acid-Dissociable Group:

Examples of acid-dissociable groups that protect a carboxy group among the above polar groups include an acid-dissociable group represented by the following General Formula (a1-r-4).

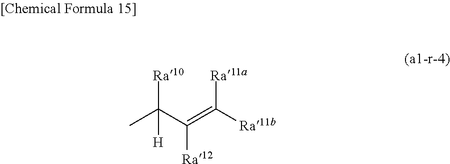

[in the formula, Ra′¹⁰ is a hydrocarbon group, Ra′^{11a} and Ra′^{11b} are each independently a hydrogen atom, a halogen atom or an alkyl group, Ra′¹² is a hydrogen atom or a hydrocarbon group, Ra′¹⁰ and Ra′^{11a} or Ra′^{11b} may be bonded to each other to form a ring, and Ra′^{11a} or Ra′^{11b} and Ra′¹² may be bonded to each other to form a ring].

In the formula, examples of hydrocarbon groups for Ra′¹⁰ and Ra′¹² include the same groups as for Ra′¹³.

In the formula, examples of alkyl groups for Ra′^{11a} and Ra′^{11b} include the same alkyl groups as for ra′¹.

In the formula, the hydrocarbon groups for Ra′¹⁰ and Ra′¹² and the alkyl groups for Ra′^{11a} and Ra′^{11b} may have a substituent. Examples of substituents include those exemplified for Ra^{x5}

Ra′¹⁰ and Ra′^{11a} or Ra′^{11b} may be bonded to each other to form a ring, and The ring may be a polycycle, a monocycle, an alicyclic ring, or an aromatic ring.

The alicyclic ring and aromatic ring may contain a heteroatom.

As the ring formed by bonding Ra′¹⁰ and Ra′^{11a} or Ra′^{11b}, among the above examples, a monocycloalkene, a ring in which some carbon atoms in a monocycloalkene are substituted with a heteroatom (an oxygen atom, a sulfur atom, etc.), and a monocycloalkadiene are preferable, a cycloalkene having 3 to 6 carbon atoms is preferable, and cyclopentene or cyclohexene is preferable.

The ring formed by bonding Ra′¹⁰ and Ra′^{11a} or Ra′^{11b} may be a fused ring. Specific examples of fused rings include indane.

The ring formed by bonding Ra′¹⁰ and Ra′^{11a} or Ra′^{11b} may have a substituent. Examples of substituents include those exemplified for Ra^{x5}.

Ra′^{11a} or Ra′^{11b} and Ra′¹² may be bonded to each other to form a ring, and examples of rings include the same rings formed by bonding Ra′¹⁰ and Ra′^{11a} or Ra′^{11b}.

Specific examples of groups represented by Formula (a1-r-4) are shown below.

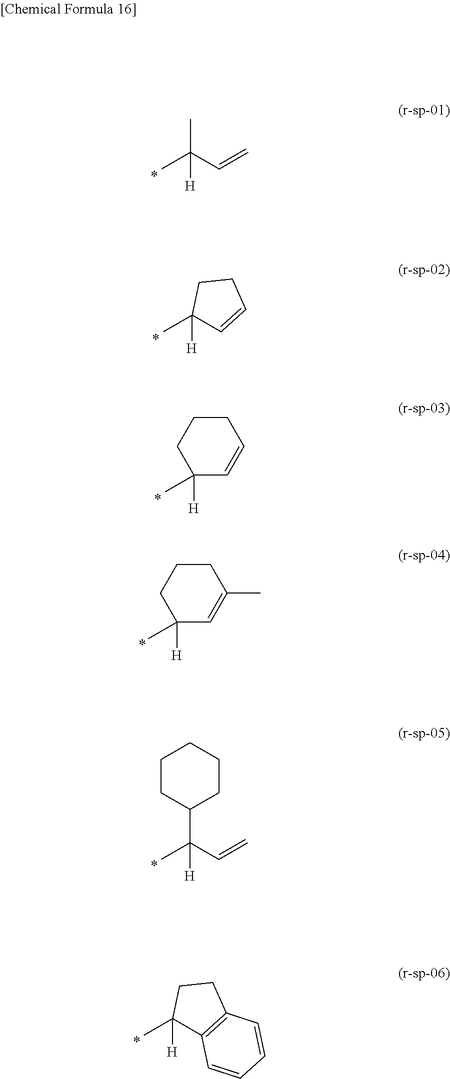

Examples of structural units (a1) include a structural unit derived from an acrylic acid ester in which the hydrogen atom bonded to the carbon atom at the α-position may be substituted with a substituent, a structural unit derived from an acrylamide, a structural unit in which at least some hydrogen atoms in the hydroxy group of a structural unit derived from hydroxystyrene or a hydroxystyrene derivative are protected by a substituent containing the acid-decomposable group, and a structural unit in which at least some hydrogen atoms in —C(═O)—OH of a structural unit derived from vinylbenzoic acid or a vinylbenzoic acid derivative are protected by a substituent containing the acid-decomposable group.

Among the above examples, the structural unit (a1) is preferably a structural unit derived from an acrylic acid ester in which the hydrogen atom bonded to the carbon atom at the α-position may be substituted with a substituent.

Preferable specific examples of such structural units (a1) include structural units represented by the following General Formula (a1-1), (a1-2) or (a1-3).

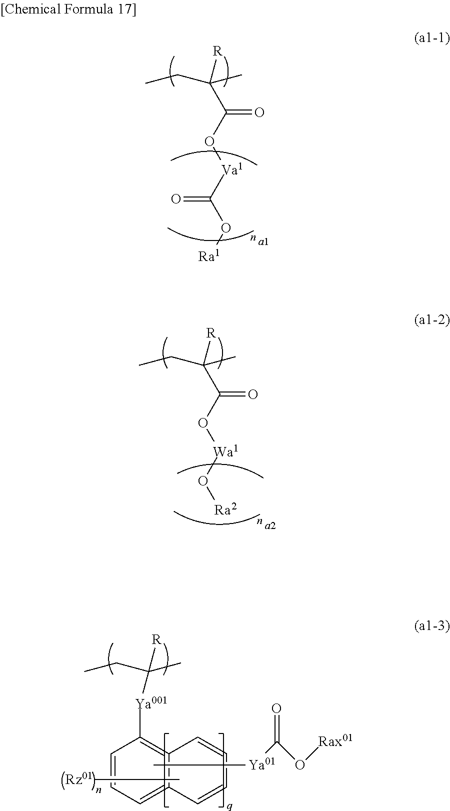

[in the formula, R is a hydrogen atom, an alkyl group having 1 to 5 carbon atoms, or a halogenated alkyl group having 1 to 5 carbon atoms, Va¹ is a divalent hydrocarbon group which may contain an ether bond, nₐ₁ is an integer of 0 to 2, Ra¹ is an acid-dissociable group represented by General Formula (a1-r-1), (a1-r-2) or (a1-r-4), Wa¹ is an (na2+1)-valent hydrocarbon group, nₐ₂ is an integer of 1 to 3, Ra² is an acid-dissociable group represented by General Formula (a1-r-1) or (a1-r-3), Ya⁰⁰¹ is a single bond or a divalent linking group, Ya⁰¹ is a single bond or a divalent linking group, Rax⁰¹ is an acid-dissociable group represented by General Formula (a1-r-1), (a1-r-2) or (a1-r-4), Rz⁰¹ is an alkyl group, a halogen atom, a halogenated alkyl group, hydroxy group, or an alkoxy group, q is an integer of 0 to 3, and n is an integer of 0 or more, where n≤q×2+4].

In Formulae (a1-1) to (a1-3), the alkyl group having 1 to 5 carbon atoms for R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which some or all of the hydrogen atoms in the alkyl group having 1 to 5 carbon atoms are substituted with halogen atoms. The halogen atom is particularly preferably a fluorine atom.

R is preferably a hydrogen atom, an alkyl group having 1 to 5 carbon atoms or a fluorinated alkyl group having 1 to 5 carbon atoms, and in terms of industrial availability, a hydrogen atom or a methyl group is most preferable.

In Formula (a1-1), the divalent hydrocarbon group for Va¹ may be an aliphatic hydrocarbon group or an aromatic hydrocarbon group.

The aliphatic hydrocarbon group as the divalent hydrocarbon group for Va¹ may be saturated or unsaturated, and is generally preferably saturated.

More specific examples of aliphatic hydrocarbon groups include a linear or branched aliphatic hydrocarbon group and an aliphatic hydrocarbon group containing a ring in the structure.

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Examples of aliphatic hydrocarbon groups containing a ring in the structure include an alicyclic hydrocarbon group (a group in which two hydrogen atoms have been removed from an aliphatic hydrocarbon ring), a group in which an alicyclic hydrocarbon group is bonded to the end of a linear or branched aliphatic hydrocarbon group, and a group in which an alicyclic hydrocarbon group is inserted into a linear or branched aliphatic hydrocarbon group. Examples of linear or branched aliphatic hydrocarbon groups include the same linear aliphatic hydrocarbon groups or branched aliphatic hydrocarbon groups.

The alicyclic hydrocarbon group preferably has 3 to 20 carbon atoms and more preferably has 3 to 12 carbon atoms.

The alicyclic hydrocarbon group may be polycyclic or monocyclic. The monocyclic alicyclic hydrocarbon group is preferably a group in which two hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms have been removed from a polycycloalkane, the polycycloalkane is preferably one having 7 to 12 carbon atoms, and specific examples thereof include adamantane, norbornane, isobornane, tricyclo[5.2.1.0^{2,6}]decane, and tetracyclododecane.

The aromatic hydrocarbon group as the divalent hydrocarbon group for Va¹ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. Here, the number of carbon atoms does not include the number of carbon atoms in the substituent.

Specific examples of aromatic rings in the aromatic hydrocarbon group include aromatic hydrocarbon rings such as benzene, biphenyl, fluorene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom.

Specific examples of aromatic hydrocarbon groups include a group in which two hydrogen atoms have been removed from the aromatic hydrocarbon ring (arylene group); and a group in which one hydrogen atom of a group (aryl group) in which one hydrogen atom has been removed from the aromatic hydrocarbon ring is substituted with an alkylene group (for example, a group in which one hydrogen atom has been additionally removed from the aryl group in the arylalkyl group such as a benzyl group, a phenethyl group, a 1-naphthyl methyl group, a 2-naphthyl methyl group, a 1-naphthyl ethyl group, and a 2-naphthyl ethyl group). The alkylene group (the alkyl chain in the arylalkyl group) preferably has 1 to 4 carbon atoms, more preferably has 1 or 2 carbon atoms, and particularly preferably has 1 carbon atom.

In Formula (a1-1), Ra¹ is preferably an acid-dissociable group represented by General Formula (a1-r-2) or (a1-r-4), and among these, a group represented by General Formula (a1-r2-1) or an acid-dissociable group represented by General Formula (a1-r-4) is more preferable.

In Formula (a1-2), the (nₐ₂+1)-valent hydrocarbon group for Wa¹ may be an aliphatic hydrocarbon group or an aromatic hydrocarbon group. The aliphatic hydrocarbon group is a hydrocarbon group having no aromaticity, and may be saturated or unsaturated, and is generally preferably saturated. Examples of aliphatic hydrocarbon groups include a linear or branched aliphatic hydrocarbon group, an aliphatic hydrocarbon group containing a ring in the structure, and a group in which a linear or branched aliphatic hydrocarbon group and an aliphatic hydrocarbon group containing a ring in the structure are combined.

The valence of nₐ₂+1 is preferably 2 to 4 and more preferably 2 or 3.

In Formula (a1-2), Ra² is preferably an acid-dissociable group represented by General Formula (a1-r-1).

In Formula (a1-3), the divalent linking group for Ya⁰⁰¹ is not particularly limited, and preferable examples thereof include a divalent hydrocarbon group which may have a substituent and a heteroatom-containing divalent linking group.

Ya⁰⁰¹ is preferably an ester bond [—C(═O)—O—, —O—C(═O)—], an ether bond (—O—), a linear or branched alkylene group, an aromatic hydrocarbon group or a combination thereof, or a single bond. The alkylene group preferably has 1 to 10 carbon atoms, more preferably has 1 to 6 carbon atoms, still more preferably has 1 to 4 carbon atoms, and particularly preferably has 1 to 3 carbon atoms.

Among these, Ya⁰⁰¹ is more preferably a combination of an ester bond [—C(═O)—O—, —O—C(═O)—] and a linear alkylene group or a single bond, and still more preferably a single bond.

In Formula (a1-3), the divalent linking group for Ya⁰¹ is not particularly limited, and preferable examples thereof include a divalent hydrocarbon group which may have a substituent and a heteroatom-containing divalent linking group.

Among the above examples, Ya⁰¹ is preferably an ester bond [—C(═O)—O—, —O—C(═O)—], an ether bond (—O—), a linear or branched alkylene group, an aromatic hydrocarbon group or a combination thereof, or a single bond. Among these, Ya⁰¹ is more preferably a combination of an ester bond [—C(═O)—O—, —O—C(═O)—] and a linear alkylene group or a single bond, and still more preferably a single bond.

In Formula (a1-3), Rax⁰¹ is preferably an acid-dissociable group represented by General Formula (a1-r-2) or (a1-r-4), and among these, an acid-dissociable group represented by General Formula (a1-r-2) is more preferable, and a group represented by General Formula (a1-r2-1) is still more preferable.

In Formula (a1-3), the alkyl group, halogenated alkyl group, and alkoxy group for Rz⁰¹ preferably has 1 to 10 carbon atoms, more preferably has 1 to 5 carbon atoms, still more preferably has 1 to 3 carbon atoms, and particularly preferably has 1 or 2 carbon atoms. The alkyl group, halogenated alkyl group, and alkoxy group may be linear or branched

The halogen atom for Rz⁰¹ is preferably an iodine atom. The halogen atom in the halogenated alkyl group for Rz⁰¹ is preferably a fluorine atom, an iodine atom, or a bromine atom, and more preferably a fluorine atom.

Rz⁰¹ is preferably an alkoxy group or a hydroxy group, and more preferably a hydroxy group.

In Formula (a1-3), q is an integer of 0 to 3. A benzene structure is formed when q is 0, a naphthalene structure is formed when q is 1, an anthracene structure is formed when q is 2, and a tetracene structure is formed when q is 3.

In Formula (a1-3), n is an integer of 0 or more, preferably 0 to 5, more preferably 0 to 3, and still more preferably 1 or 2. When n is an integer of 2 or more, two or more Rz⁰¹'s may be the same as or different from each other.

In Formula (a1-3), n ≤q×2+4. For example, when q is 1 and the structure is naphthalene, all six hydrogen atoms of the naphthalene may be substituted with hydroxy groups. In addition, in the naphthalene, the substitution positions of Ya⁰⁰¹, a -Ya⁰¹—C(═O)—O—Ra⁰¹ group, and a hydroxy group are not particularly limited.

Specific examples of structural units (a1) are shown below. In the following formulae, R^{α} is a hydrogen atom, a methyl group or a trifluoromethyl group.

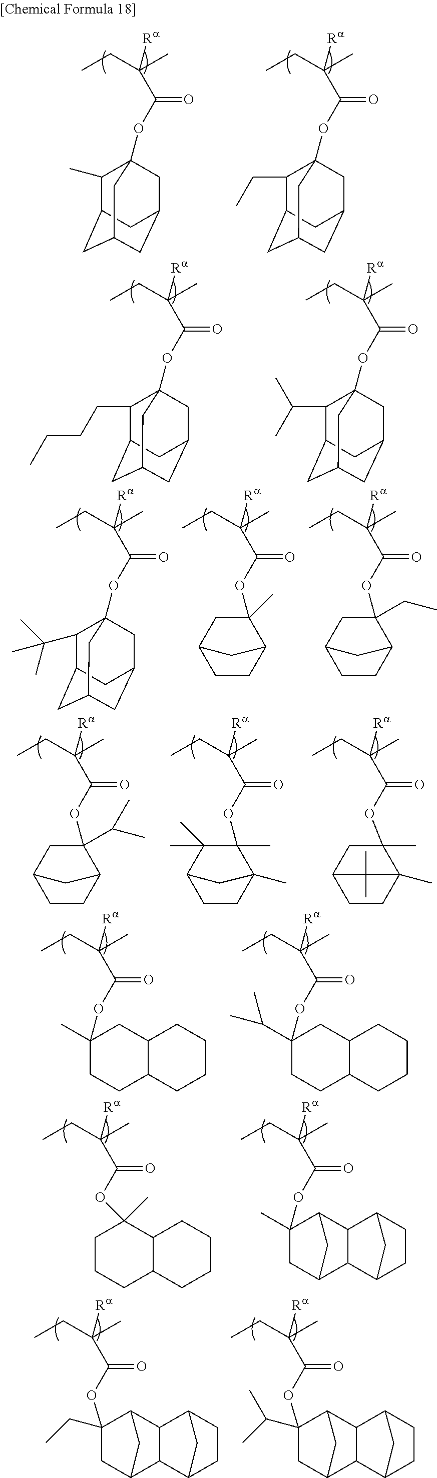

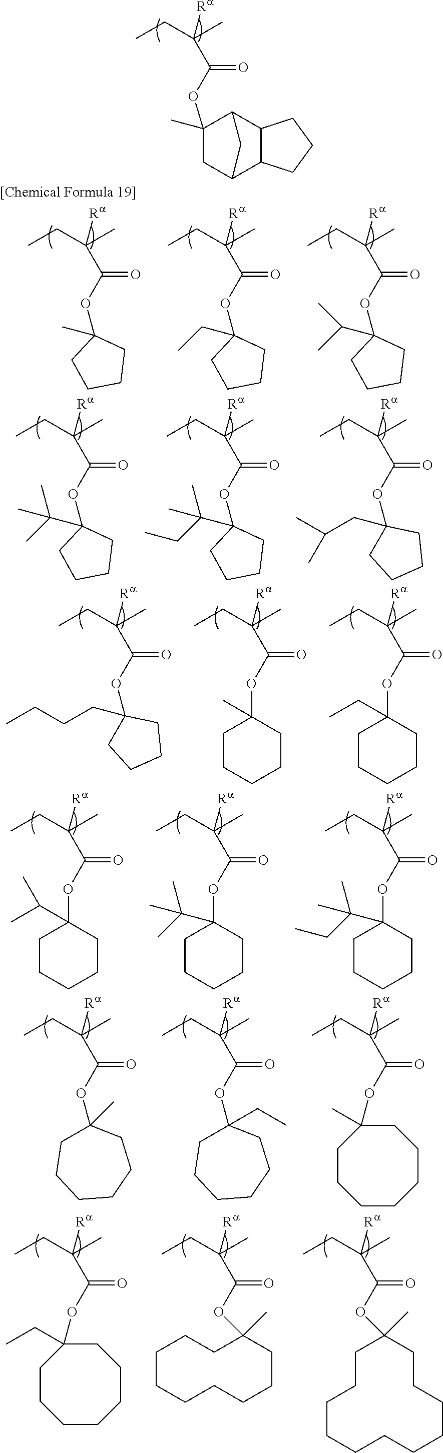

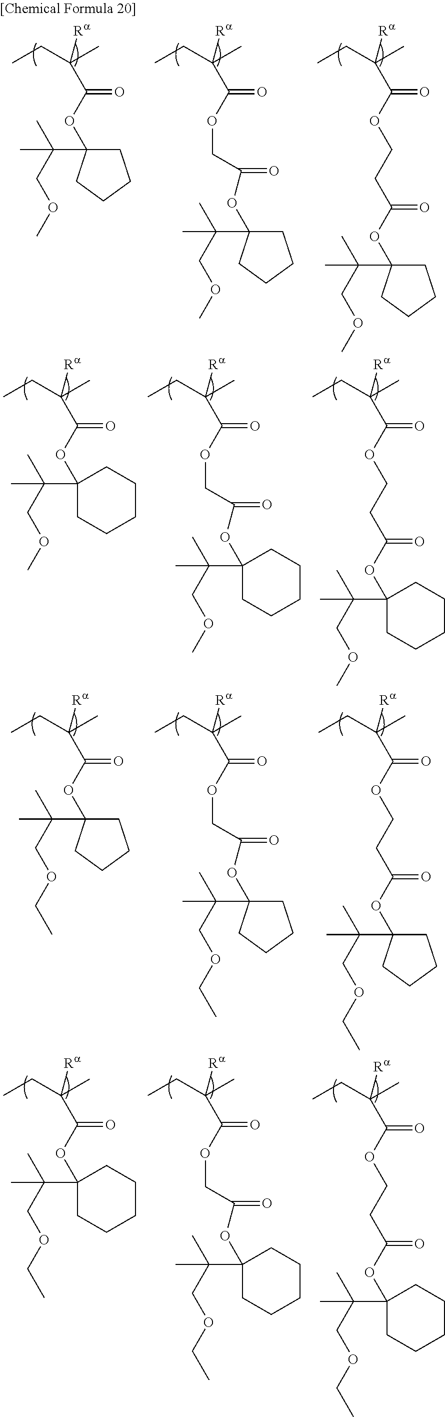

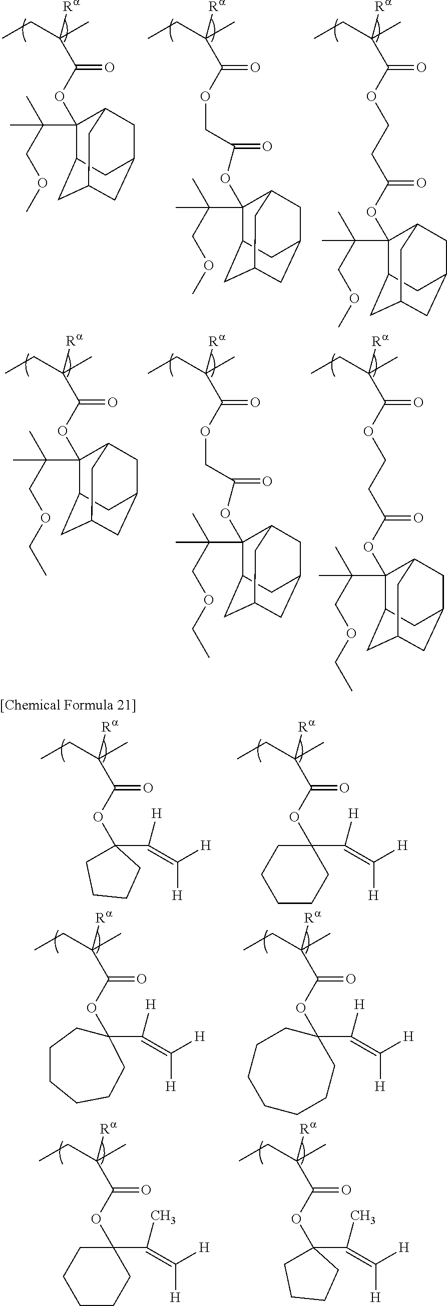

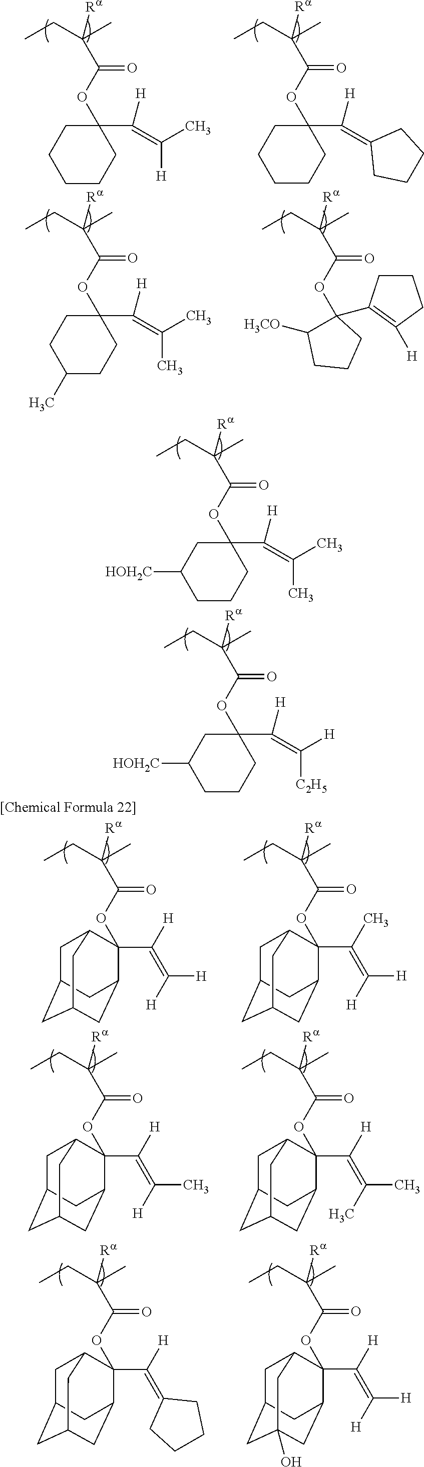

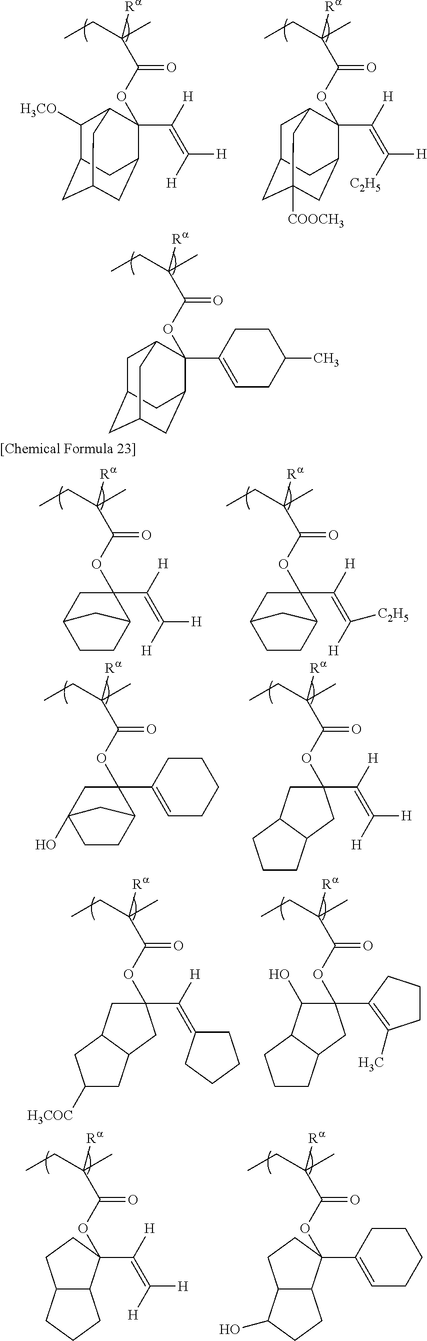

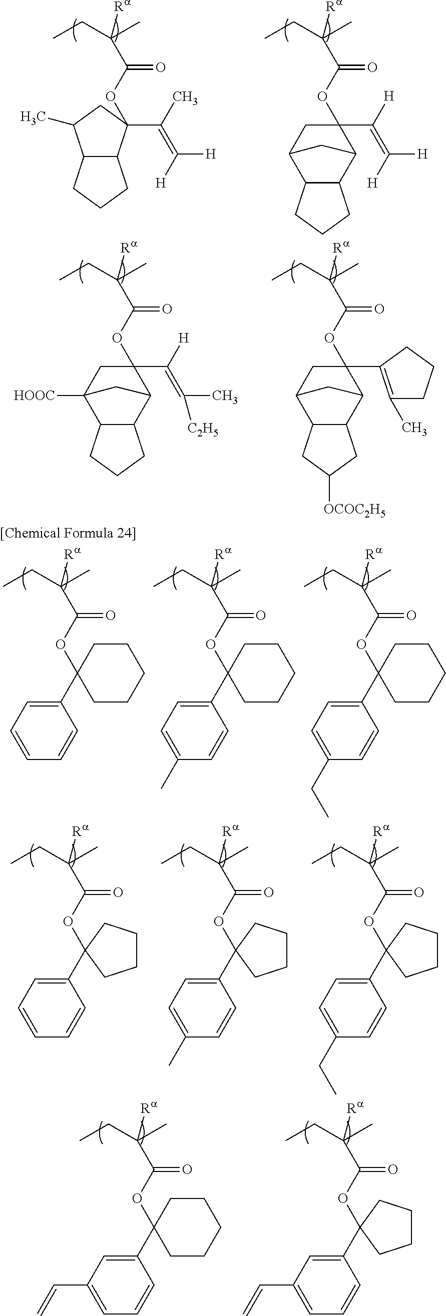

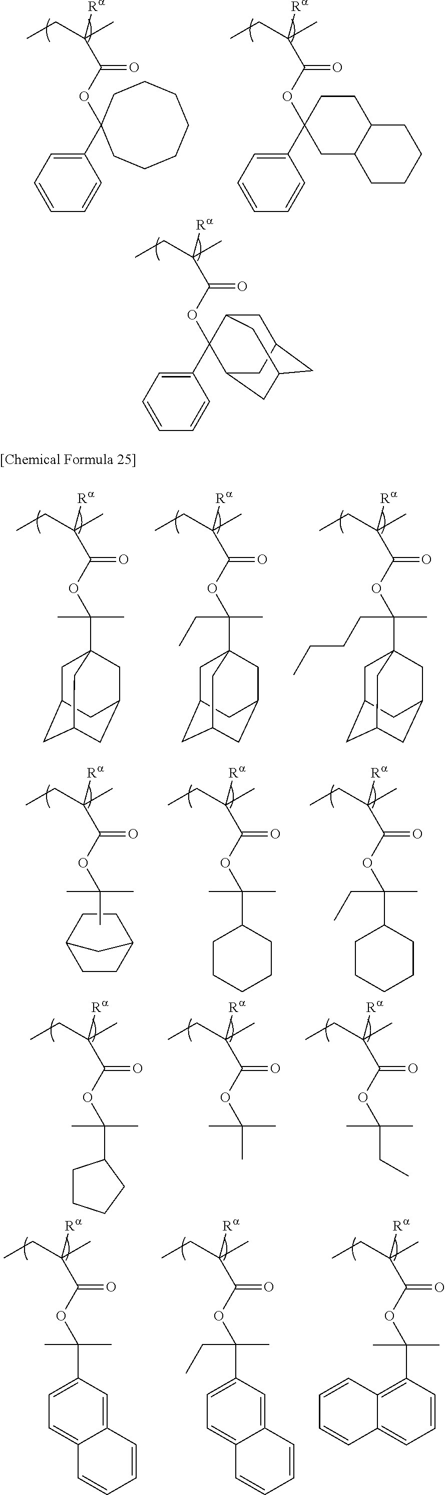

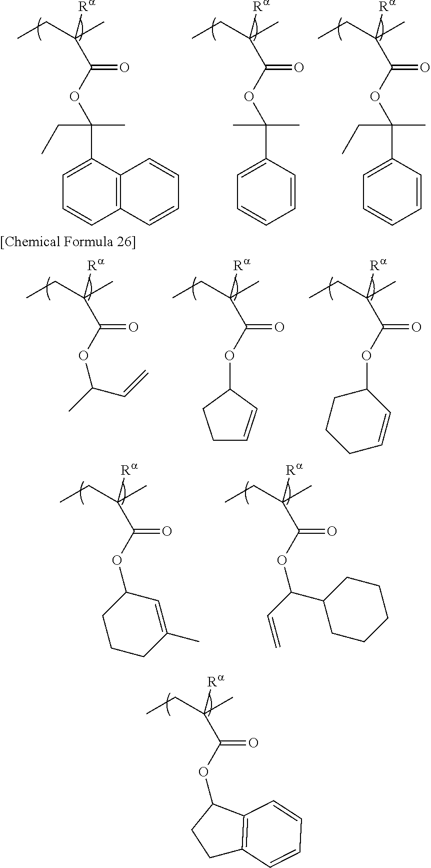

In the following formulae, R^{α} is a hydrogen atom, a methyl group or a trifluoromethyl group. Rz is a hydrogen atom, an alkyl group, a halogen atom, a halogenated alkyl group, a hydroxy group, or an alkoxy group.

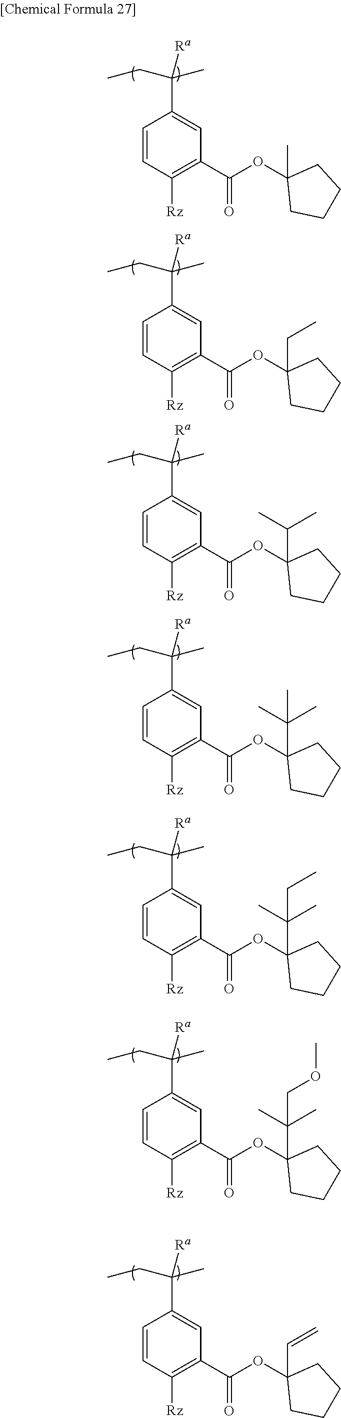

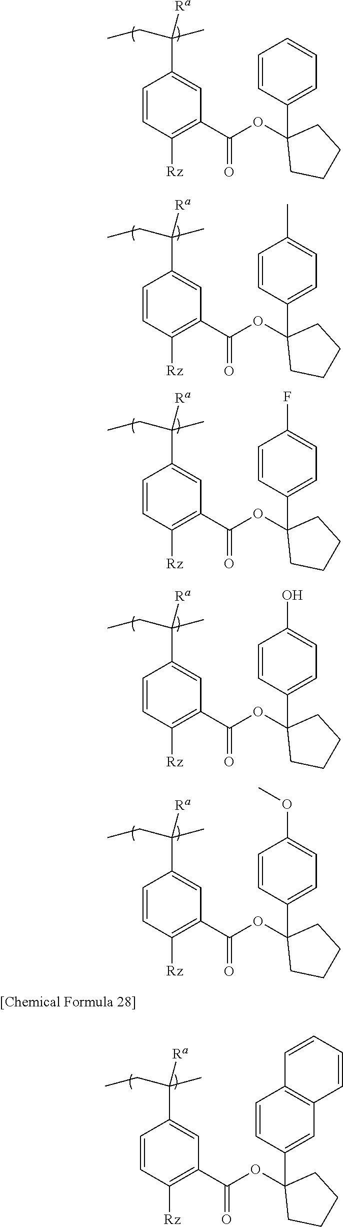

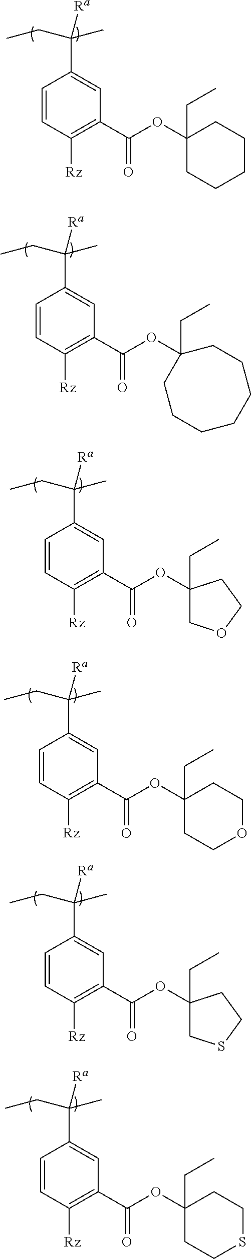

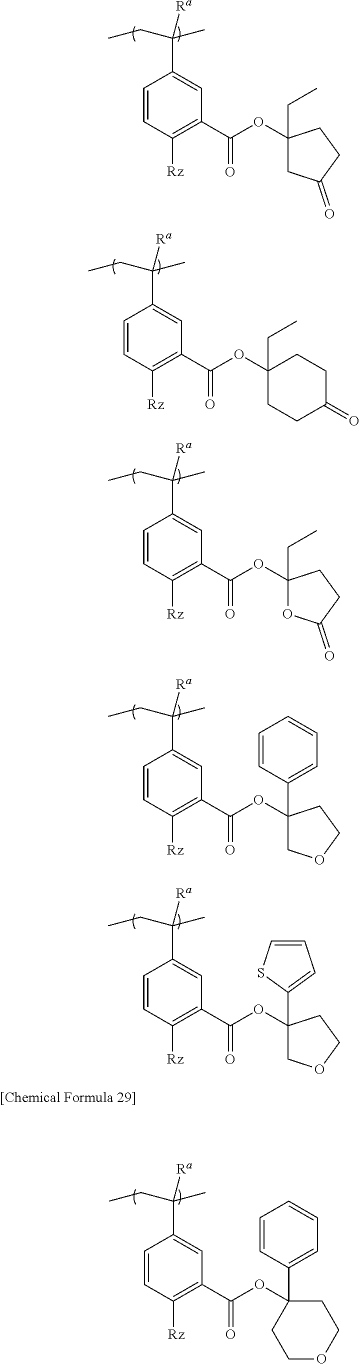

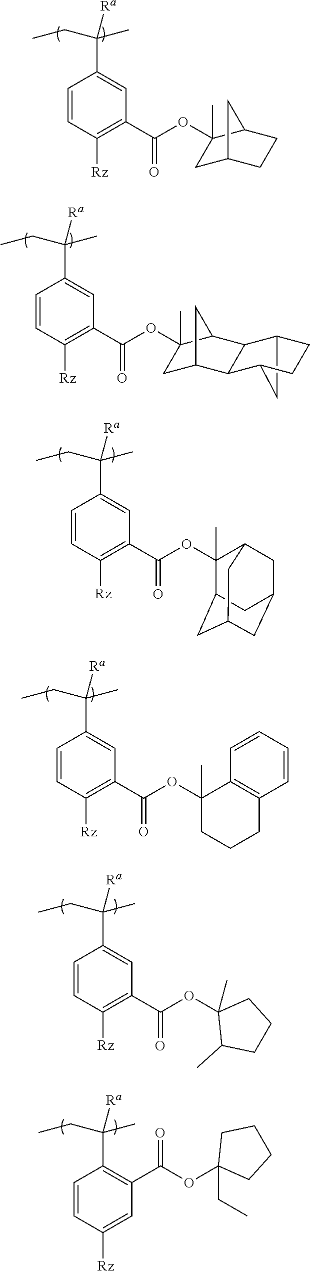

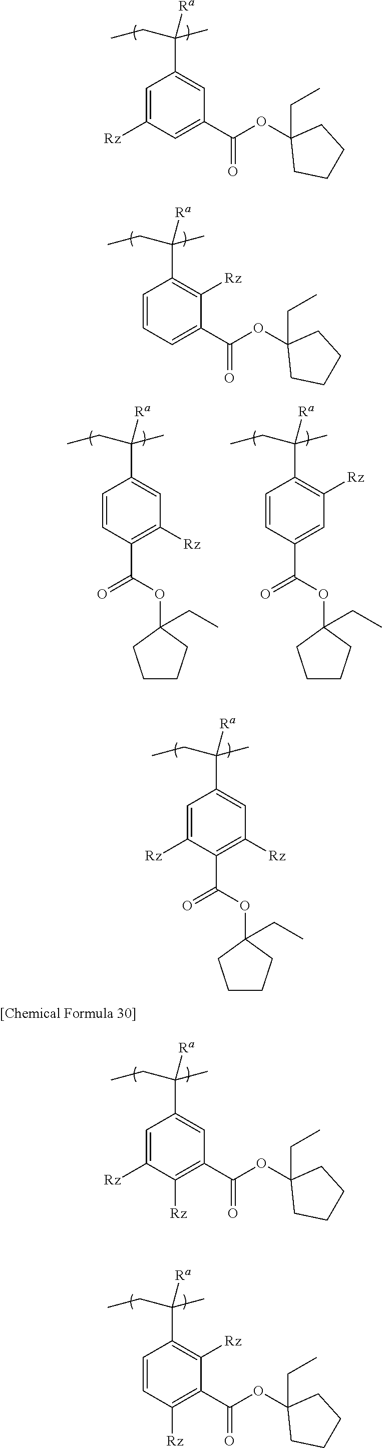

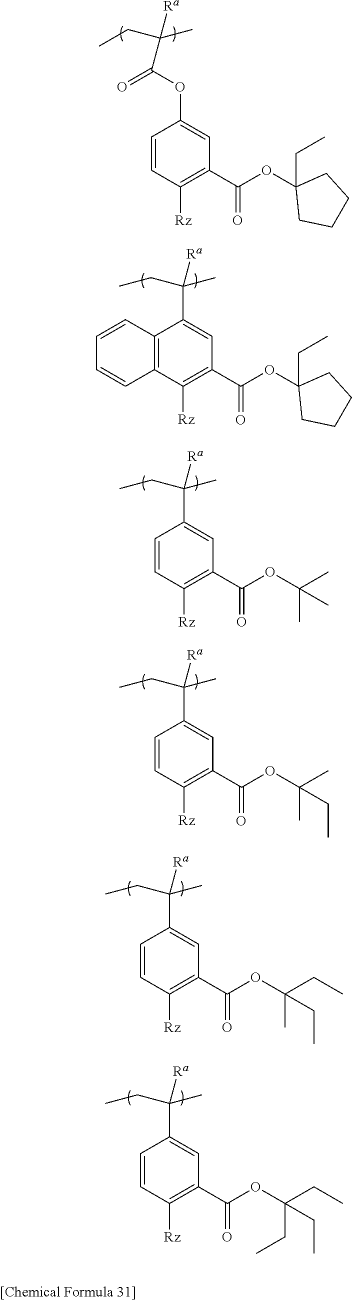

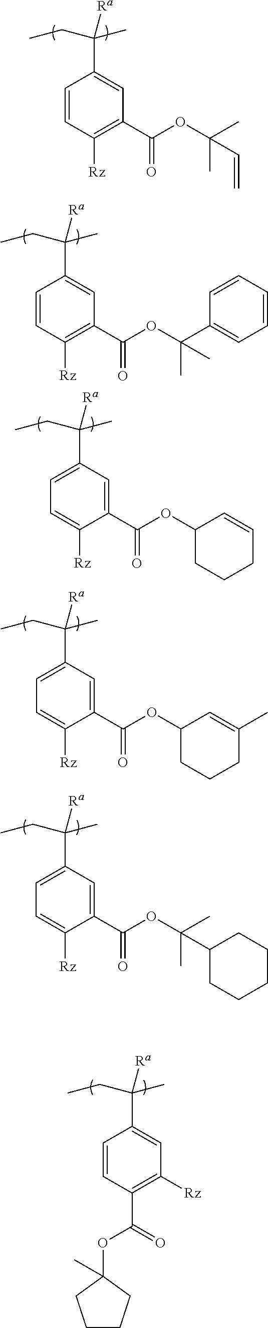

The structural unit (a1) of the component (A1) may be of one type or of two or more types.

As the structural unit (a1), the structural unit represented by Formula (a1-1) or the structural unit represented by Formula (a1-3) is more preferable because it makes it easier to further improve properties (sensitivity, shape, etc.) in electron beam or EUV lithography.

Among these, the acid-dissociable groups (Ra¹ and Rax⁰¹) are each preferably an acid-dissociable group represented by General Formula (a1-r2-1), (a1-r2-3), (a1-r2-4) or (a1-r-4) because it is suitable to improve the reactivity with EB or EUV, and among these, it is particularly preferable to select the acid-dissociable group that is a cyclic group.

Alternatively, the structural unit (a1) may include a structural unit represented by the following General Formula (a1-1-1).

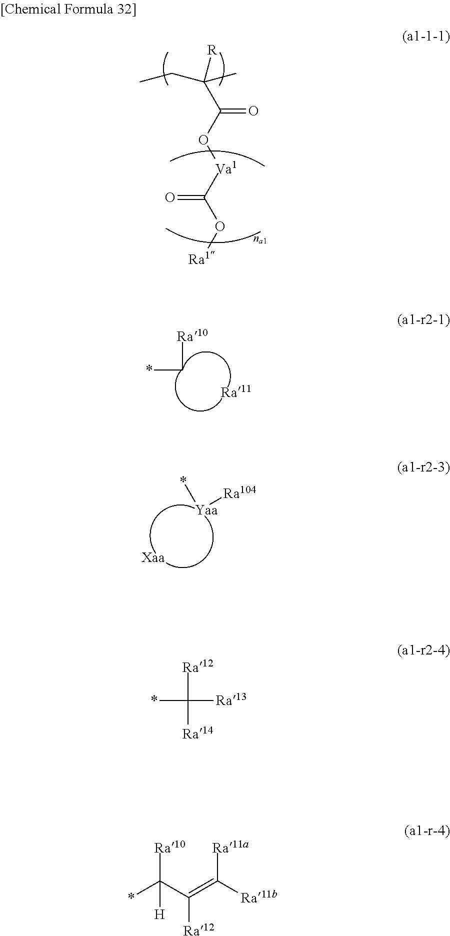

[in the formula, Ra¹″ is an acid-dissociable group represented by General Formula (a1-r2-1), (a1-r2-3), (a1-r2-4) or (a1-r-4), and indicates a bond].

In Formula (a1-1-1), R, Va¹ and nₐ₁ are the same as R, Va¹ and nₐ₁ in Formula (a1-1).

The acid-dissociable group represented by General Formula (a1-r2-1), (a1-r2-3), (a1-r2-4) or (a1-r-4) is described as above. Among these, it is preferable to select the acid-dissociable group that is a cyclic group because it is suitable to improve the reactivity with EB or EUV.

The proportion of the structural unit (a1) in the component (A1) based on a total amount (100 mol %) of all structural units constituting the component (A1) is preferably 5 to 80 mol %, more preferably 10 to 75 mol %, still more preferably 30 to 70 mol %, and particularly preferably 40 to 70 mol %.

When the proportion of the structural unit (a1) is set to be equal to or larger than the lower limit value of the preferable range, lithography properties such as sensitivity, resolution, and CDU are improved. On the other hand, when the proportion is set to be equal to or smaller than the upper limit value of the preferable range, a balance with other structural units can be achieved and various lithography properties become favorable.

### <<Other Structural Units>>

The component (A1) may have, as necessary, other structural units, in addition to the above structural unit (a1).

Examples of other structural units include a structural unit (a10) represented by the following General Formula (a10-1); a structural unit having a lactone-containing cyclic group (a2); a structural unit (a5) that generates an acid upon exposure; a structural unit (a6) having an ability to control acid diffusion; and a structural unit (a8) derived from a compound represented by the following General Formula (a8-1).

Structural Unit (a10):

The structural unit (a10) is a structural unit represented by the following General Formula (a10-1).

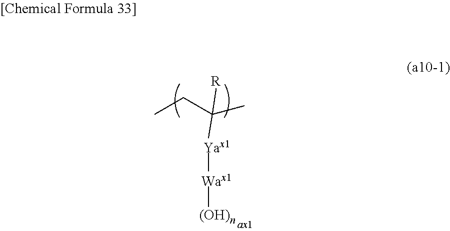

[in the formula, R is a hydrogen atom, an alkyl group having 1 to 5 carbon atoms or a halogenated alkyl group having 1 to 5 carbon atoms, Ya^{x1} is a single bond or a divalent linking group, Wa^{x1} is an aromatic hydrocarbon group which may have a substituent, and nₐₓ₁ is an integer of 1 or more].

In Formula (a10-1), R is the same as R in General Formula (a1-1). R is preferably a hydrogen atom, an alkyl group having 1 to 5 carbon atoms or a fluorinated alkyl group having 1 to 5 carbon atoms, and in terms of industrial availability, a hydrogen atom or a methyl group is particularly preferable.

In Formula (a10-1), Ya^{x1} is a single bond or a divalent linking group,

In the chemical formula, the divalent linking group for Ya^{x1} is not particularly limited, and preferable examples thereof include a divalent hydrocarbon group which may have a substituent and a heteroatom-containing divalent linking group.

Divalent hydrocarbon group which may have substituent:

The divalent hydrocarbon group which may have a substituent may be an aliphatic hydrocarbon group or an aromatic hydrocarbon group.

### Aliphatic Hydrocarbon Group

The aliphatic hydrocarbon group is a hydrocarbon group having no aromaticity. The aliphatic hydrocarbon group may be saturated or unsaturated, and is generally preferably saturated.

Examples of aliphatic hydrocarbon groups include a linear or branched aliphatic hydrocarbon group and an aliphatic hydrocarbon group containing a ring in the structure.

### Linear or Branched Aliphatic Hydrocarbon Group

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The linear or branched aliphatic hydrocarbon group may or may not have a substituent. Examples of substituents include a fluorine atom, a fluorinated alkyl group having 1 to 5 carbon atoms and substituted with a fluorine atom, and a carbonyl group.

### Aliphatic Hydrocarbon Group Containing Ring in Structure

Examples of aliphatic hydrocarbon groups containing a ring in the structure include a cyclic aliphatic hydrocarbon group which may contain a heteroatom-containing substituent in the ring structure (a group in which two hydrogen atoms have been removed from an aliphatic hydrocarbon ring), a group in which the cyclic aliphatic hydrocarbon group is bonded to the end of a linear or branched aliphatic hydrocarbon group, and a group in which the cyclic aliphatic hydrocarbon group is inserted into a linear or branched aliphatic hydrocarbon group. Examples of linear or branched aliphatic hydrocarbon groups include the same groups described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group in which two hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms have been removed from a polycycloalkane, the polycycloalkane is preferably one having 7 to 12 carbon atoms, and specific examples thereof include adamantane, norbornane, isobornane, tricyclo[5.2.1.0^{2,6}]decane, and tetracyclododecane.

The cyclic aliphatic hydrocarbon group may or may not have a substituent. Examples of substituents include an alkyl group, an alkoxy group, a halogen atom, a halogenated alkyl group, a hydroxy group, and a carbonyl group.

The alkyl group as the substituent is preferably an alkyl group having 1 to 5 carbon atoms and more preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group or an ethoxy group.

The halogen atom as the substituent is preferably a fluorine atom.

Examples of halogenated alkyl groups as the substituent include a group in which some or all of the hydrogen atoms in the alkyl group are substituted with a halogen atom.

In the cyclic aliphatic hydrocarbon group, some carbon atoms constituting the ring structure may be substituted with a heteroatom-containing substituent. The heteroatom-containing substituent is preferably —O—, —C(═O)—O—, —S—, —S(═O)₂—, or —S(═O)₂—O—.

### Aromatic Hydrocarbon Group

The aromatic hydrocarbon group is a hydrocarbon group having at least one aromatic ring.

The aromatic ring is not particularly limited as long as it is a cyclic conjugated system having 4n+2 π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms does not include the number of carbon atoms in the substituent.

Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

Specific examples of aromatic hydrocarbon groups include a group in which two hydrogen atoms have been removed from the aromatic hydrocarbon ring or aromatic heterocycle (an arylene group or a heteroarylene group); a group in which two hydrogen atoms have been removed from an aromatic compound containing two or more aromatic rings (for example, biphenyl, fluorene, etc.); and a group in which one hydrogen atom of a group (an aryl group or a heteroaryl group) in which one hydrogen atom has been removed from the aromatic hydrocarbon ring or aromatic heterocycle is substituted with an alkylene group (for example, a group in which one hydrogen atom has been additionally removed from the aryl group in the arylalkyl group such as a benzyl group, a phenethyl group, a 1-naphthyl methyl group, a 2-naphthyl methyl group, a 1-naphthyl ethyl group, and a 2-naphthyl ethyl group). The alkylene group bonded to the aryl group or heteroaryl group preferably has 1 to 4 carbon atoms, more preferably has 1 or 2 carbon atoms, and particularly preferably has 1 carbon atom.

In the aromatic hydrocarbon group, the hydrogen atom in the aromatic hydrocarbon group may be substituted with a substituent. For example, the hydrogen atom in the aromatic hydrocarbon group, which is bonded to an aromatic ring, may be substituted with a substituent. Examples of substituents include an alkyl group, an alkoxy group, a halogen atom, a halogenated alkyl group, and a hydroxy group.

The alkyl group as the substituent is preferably an alkyl group having 1 to 5 carbon atoms and more preferably a methyl group, an ethyl group, a propyl group, an n-butyl group, or a tert-butyl group.

Examples of alkoxy groups, halogen atoms and halogenated alkyl groups as the substituents include those exemplified as the substituent that substitutes a hydrogen atom in the cyclic aliphatic hydrocarbon group.

### Heteroatom-Containing Divalent Linking Group:

Examples of heteroatom-containing divalent linking groups include —O—, —C(═O)—O—, —O—C(═O)—, —C(═O)—, —O—C(═O)—O—, —C(═O)—NH—, —NH—, —NH—C(═NH)—(H may be substituted with a substituent such as an alkyl group or an acyl group), —S—, —S(═O)₂—, —S(═O)₂—O—, and a group represented by General Formula —Y²¹—O—Y²²—, —Y²¹—O—, —Y²¹—C(═O)—O—, —C(═O)—O—Y²¹—, —[Y²¹—C(═O)—O]_{m″}—Y²²—, —Y²¹—O—C(═O)—Y²²— or —Y²¹—S(═O)₂—O—Y²²— [in the formula, Y²¹ and Y²² are each independently a divalent hydrocarbon group which may have a substituent, O is an oxygen atom, and m″ is an integer of 1 to 3].

When the heteroatom-containing divalent linking group is —C(═O)—NH—, —C(═O)—NH—C(═O)—, —NH—, or —NH—C(═NH)—, H may be substituted with a substituent such as an alkyl group or an acyl group. The substituent (an alkyl group, an acyl group, etc.) preferably has 1 to 10 carbon atoms, more preferably has 1 to 8 carbon atoms, and particularly preferably has 1 to 5 carbon atoms.

In General Formula —Y²¹—O—Y²²—, —Y²¹—O—, —Y²¹—C(═O)—O—, —C(═O)—O—Y²¹—, —[Y²¹—C(═O)—O]_{m″}-Y²²_, —Y²¹—O—C(═O)—Y²²— or —Y²¹—S(═O)²—O—Y²²—, Y²¹ and Y²² are each independently a divalent hydrocarbon group which may have a substituent. Examples of divalent hydrocarbon groups include the same as those described above.

Y²¹ is preferably a linear aliphatic hydrocarbon group, more preferably a linear alkylene group, still more preferably a linear alkylene group having 1 to 5 carbon atoms, and particularly preferably a methylene group or an ethylene group.

Y²² is preferably a linear or branched aliphatic hydrocarbon group and more preferably a methylene group, an ethylene group or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by Formula —[Y²¹—C(═O)—O]_{m″}—Y²²—, m″ is an integer of 1 to 3, preferably an integer of 1 or 2, and more preferably 1. That is, as the group represented by Formula —[Y²¹—C(═O)—O]_{m″}—Y—Y²²—, a group represented by Formula —Y²¹—C(═O)—O—Y²²— is particularly preferable. Among these, a group represented by Formula —(CH₂)_{a′}—C(═O)—O—(CH₂)_{b′}— is preferable. In the formula, a′ is an integer of 1 to 10, preferably an integer of 1 to 8, more preferably an integer of 1 to 5, still more preferably 1 or 2, and most preferably 1. b′ is an integer of 1 to 10, preferably an integer of 1 to 8, more preferably an integer of 1 to 5, still more preferably 1 or 2, and most preferably 1.

Ya^{x1} is preferably a single bond, an ester bond [—C(═O)—O—, —O—C(═O)—], an ether bond (—O—), a linear or branched alkylene group, or a combination thereof, and more preferably a single bond or an ester bond [—C(═O)—O—, —O—C(═O)—].

In Formula (a10-1), Wa^{x1} is an aromatic hydrocarbon group which may have a substituent.

Examples of aromatic hydrocarbon groups for Wa^{x1} include a group in which (nₐₓ₁+1) hydrogen atoms have been removed from an aromatic ring which may have a substituent. Here, the aromatic ring is not particularly limited as long as it is a cyclic conjugated system having 4n+2 π electrons. The aromatic ring preferably has 5 to 30 carbon atoms, more preferably has 5 to 20 carbon atoms, still more preferably has 6 to 15 carbon atoms, and particularly preferably has 6 to 12 carbon atoms. Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

In addition, examples of aromatic hydrocarbon groups for Wa^{x1} include a group in which (nₐₓ₁+1) hydrogen atoms have been removed from an aromatic compound (for example, biphenyl, fluorine, etc.) containing an aromatic ring which may have two or more substituents.

Among the above examples, Wa^{x1} is preferably a group in which (nₐₓ₁+1) hydrogen atoms have been removed from benzene, naphthalene, anthracene or biphenyl, more preferably a group in which (nₐₓ₁+1) hydrogen atoms have been removed from benzene or naphthalene, and still more preferably a group in which (nₐₓ₁+1) hydrogen atoms have been removed from benzene.

The aromatic hydrocarbon group for Wa^{x1} may or may not have a substituent. Examples of substituents include an alkyl group, an alkoxy group, a halogen atom, and a halogenated alkyl group. Examples of alkyl groups, alkoxy groups, halogen atoms, and halogenated alkyl groups as the substituents include the same as those exemplified as the substituents for the cyclic aliphatic hydrocarbon groups for Ya^{x1}. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group. The aromatic hydrocarbon group for Wa^{x1} preferably has no substituent.

In Formula (a10-1), nₐₓ₁ is an integer of 1 or more, preferably an integer of 1 to 10, more preferably an integer of 1 to 5, still more preferably 1, 2 or 3, and particularly preferably 1 or 2.

Specific examples of structural units (a10) represented by Formula (a10-1) are shown below.

In the following formulae, R^{α} is a hydrogen atom, a methyl group or a trifluoromethyl group.

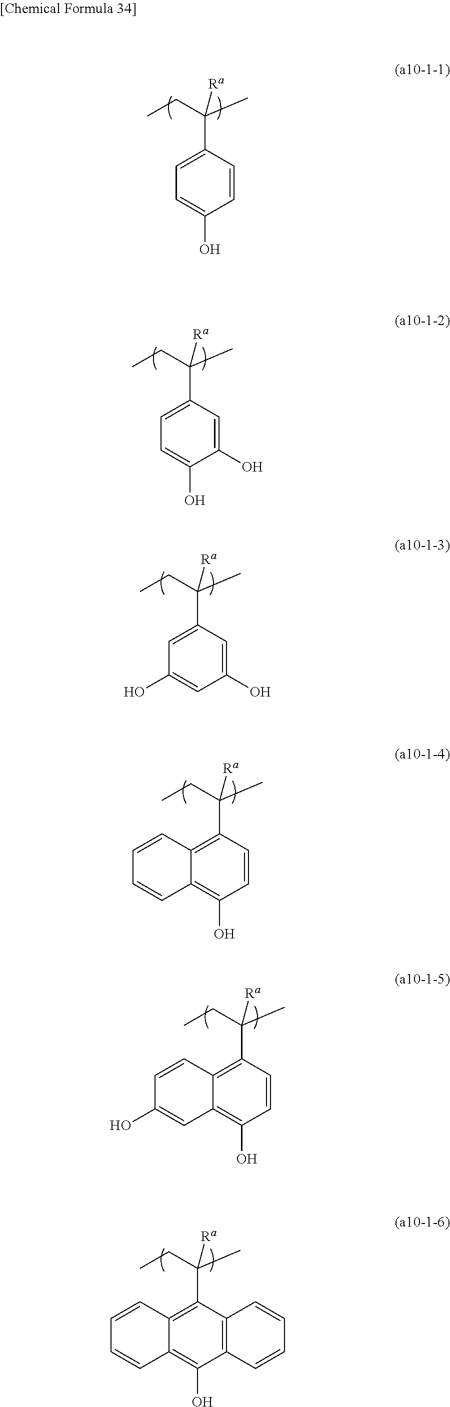

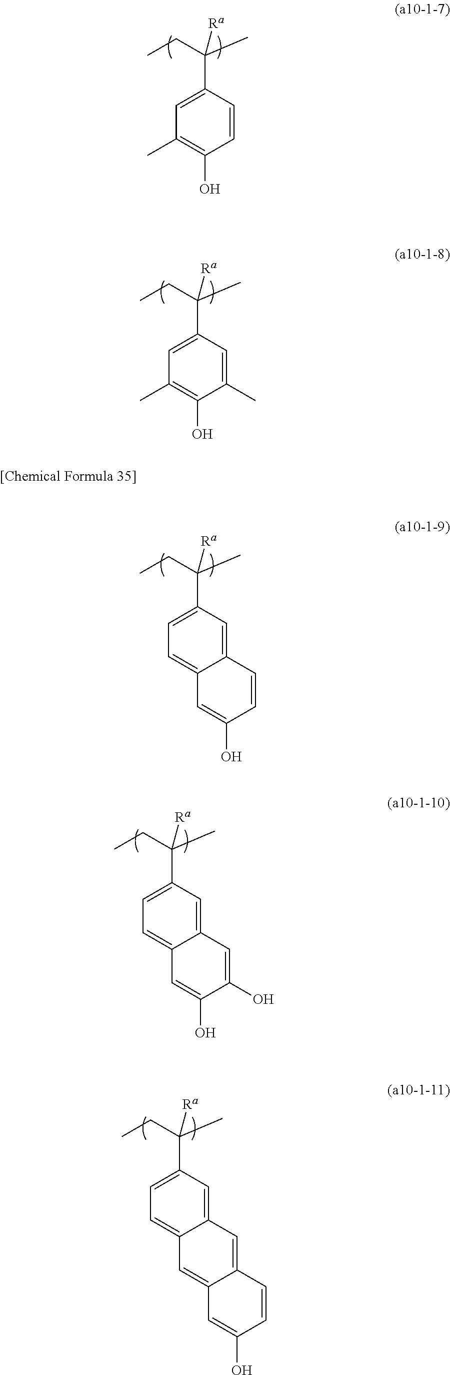

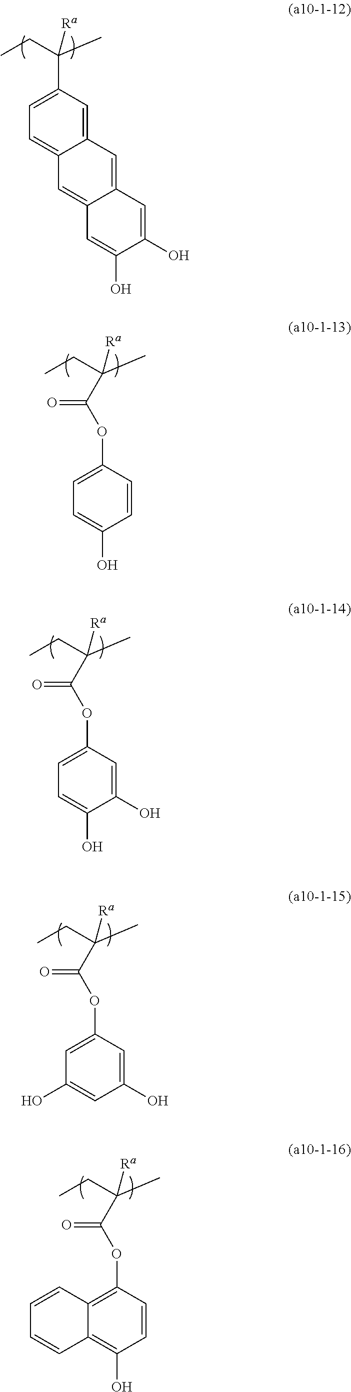

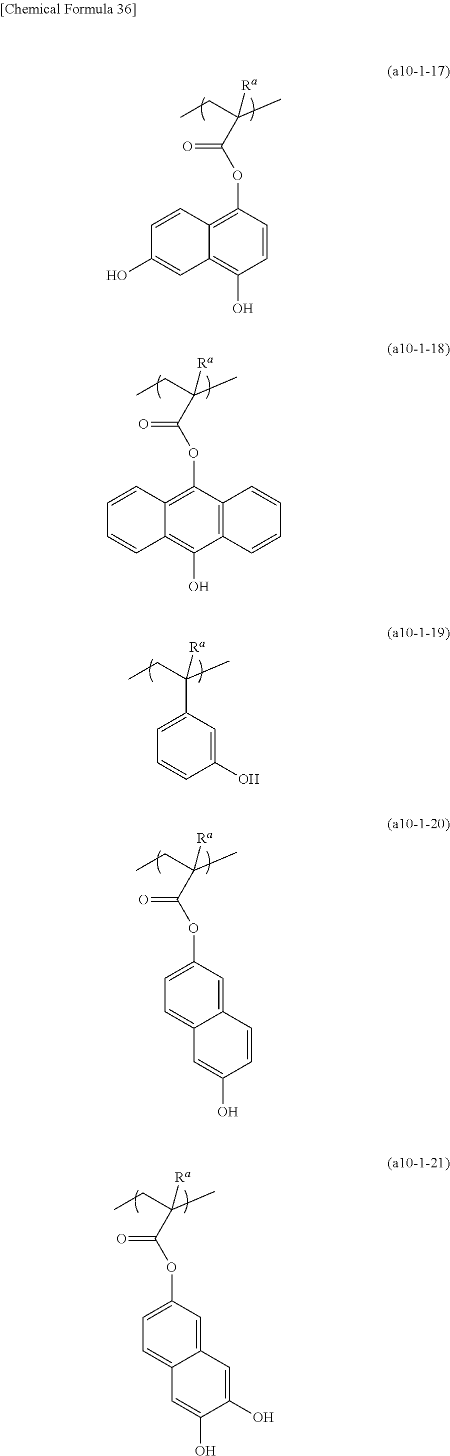

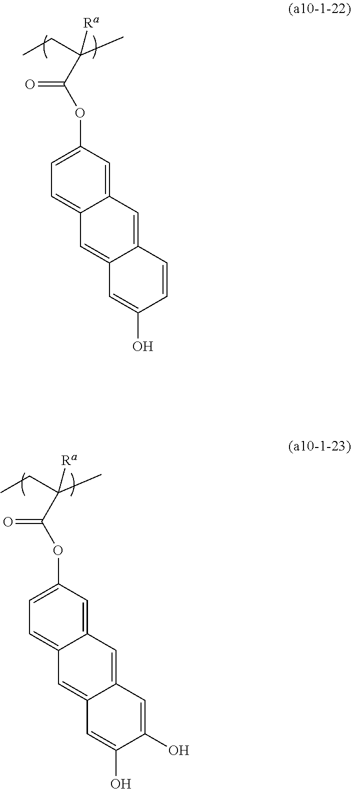

The structural unit (a10) of the component (A1) may be of one type or of two or more types. The component (A1) may or may not have the structural unit (a10) and preferably has the structural unit (a10).

When the component (A1) has the structural unit (a10), the proportion of the structural unit (a10) in the component (A1) based on a total amount (100 mol %) of all structural units constituting the component (A1) is preferably 20 to 80 mol %, more preferably 25 to 70 mol %, still more preferably 30 to 60 mol %, and particularly preferably 30 to 50 mol %.

When the proportion of the structural unit (a10) is set to be equal to or larger than the lower limit value, it is easier to further improve the sensitivity. On the other hand, when the proportion is set to be equal to or smaller than the upper limit value, it becomes easier to achieve a balance with other structural units.

### Structural Unit (a2):

The component (A1) may or may not have a structural unit (a2) having a lactone-containing cyclic group (excluding those that correspond to the structural unit (a1)).

When the component (A1) is used to form a resist film, the lactone-containing cyclic group of the structural unit (a2) is effective in improving the adhesion of the resist film to the substrate. In addition, when the structural unit (a2) is provided, for example, effects of appropriately adjusting the acid diffusion length, improving the adhesion of the resist film to the substrate, and appropriately adjusting the solubility during development are obtained, resulting in improved lithography properties and the like.

The term “lactone-containing cyclic group” refers to a cyclic group containing a ring containing —O—C(═O)— (lactone ring) in its ring framework. The lactone ring is counted as the first ring, and when there is only a lactone ring, it is called a monocyclic group, and when there are additional other ring structures, it is called a polycyclic group regardless of the structure. The lactone-containing cyclic group may be a monocyclic group or a polycyclic group.

The lactone-containing cyclic group in the structural unit (a2) is not particularly limited, and any lactone-containing cyclic group can be used. Specific examples thereof include groups represented by the following General Formulae (a2-r-1) to (a2-r-7).

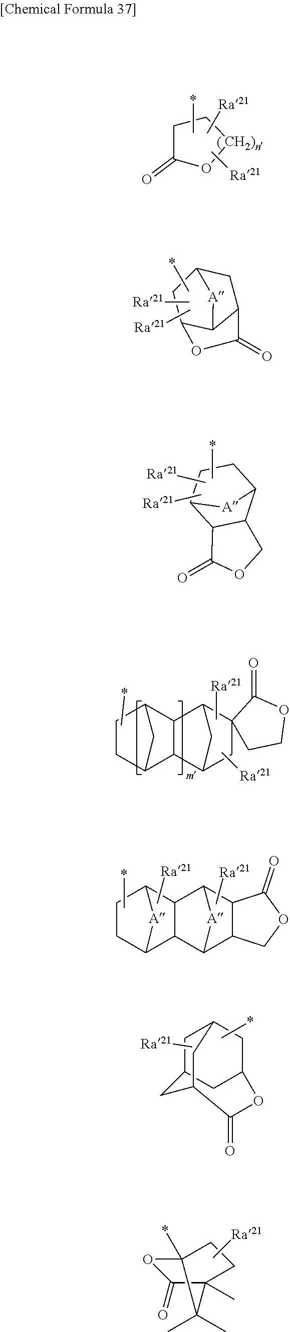

[in the formula, Ra′²¹'s are each independently a hydrogen atom, an alkyl group, an alkoxy group, a halogen atom, a halogenated alkyl group, a hydroxy group, —COOR″, —OC(═O)R″, a hydroxyalkyl group or a cyano group; R″ is a hydrogen atom, an alkyl group or a lactone-containing cyclic group; A″ is an alkylene group having 1 to 5 carbon atoms which may contain an oxygen atom (—O—) or a sulfur atom (—S—), an oxygen atom or a sulfur atom, n′ is an integer of 0 to 2, m′ is 0 or 1, and * indicates a bond (the same applies hereinafter)].

In General Formulae (a2-r-1) to (a2-r-7), the alkyl group for Ra′²¹ is preferably an alkyl group having 1 to 6 carbon atoms. The alkyl group is preferably linear or branched. Specific examples thereof include a methyl group, an ethyl group, a propyl group, an isopropyl group, an n-butyl group, an isobutyl group, a tert-butyl group, a pentyl group, an isopentyl group, a neopentyl group, and a hexyl group. Among these, a methyl group or an ethyl group is preferable, and a methyl group is particularly preferable.

The alkoxy group for Ra′²¹ is preferably an alkoxy group having 1 to 6 carbon atoms. The alkoxy group is preferably linear or branched. Specific examples thereof include a group in which an alkyl group exemplified as the alkyl group for Ra′²¹ is linked to an oxygen atom (—O—).

The halogen atom for Ra′²¹ is preferably a fluorine atom.

Examples of halogenated alkyl groups for Ra′²¹ include a group in which some or all of the hydrogen atoms in the alkyl group for Ra′²¹ are substituted with halogen atoms. The halogenated alkyl group is preferably a fluorinated alkyl group and particularly preferably a perfluoroalkyl group.

In —COOR″ and —OC(═O)R″ for Ra′²¹, R″'s are all a hydrogen atom, an alkyl group, or a lactone-containing cyclic group.

The alkyl group for R″ may be linear, branched, or cyclic, and preferably has 1 to 15 carbon atoms.

When R″ is a linear or branched alkyl group, it preferably has 1 to 10 carbon atoms, more preferably has 1 to 5 carbon atoms, and is particularly preferably a methyl group or an ethyl group.

When R″ is a cyclic alkyl group, it preferably has 3 to 15 carbon atoms, more preferably has 4 to 12 carbon atoms, and most preferably has 5 to 10 carbon atoms. Specific examples thereof include a group in which one or more hydrogen atoms have been removed from a monocycloalkane which may or may not be substituted with a fluorine atom or a fluorinated alkyl group; and a group in which one or more hydrogen atoms have been removed from a polycycloalkane such as bicycloalkane, tricycloalkane, or tetracycloalkane. More specific examples thereof include a group in which one or more hydrogen atoms have been removed from a monocycloalkane such as cyclopentane or cyclohexane; and a group in which one or more hydrogen atoms have been removed from a polycycloalkane such as adamantane, norbornane, isobornane, tricyclo[5.2.1.0^{2,6}]decane, and tetracyclododecane.

Examples of lactone-containing cyclic groups for R″ include the same groups represented by General Formulae (a2-r-1) to (a2-r-7).

The hydroxyalkyl group for Ra′²¹ preferably has 1 to 6 carbon atoms, and specific examples thereof include a group in which at least one hydrogen atom in the alkyl group for Ra′²¹ is substituted with a hydroxy group.

Among the above examples, Ra′²¹'s are each independently preferably a hydrogen atom or a cyano group.

In General Formulae (a2-r-2), (a2-r-3), and (a2-r-5), the alkylene group having 1 to 5 carbon atoms for A″ is preferably a linear or branched alkylene group, and examples thereof include a methylene group, an ethylene group, an n-propylene group, and an isopropylene group. When the alkylene group contains an oxygen atom or a sulfur atom, specific examples thereof include a group in which —O— or —S— is inserted into the end or between carbon atoms of the alkylene group, and for example, —O—CH₂—, —CH₂—O—CH₂—, —S—CH₂—, and —CH₂—S—CH₂— are exemplified. A″ is preferably an alkylene group having 1 to 5 carbon atoms or —O—, more preferably an alkylene group having 1 to 5 carbon atoms, and most preferably a methylene group.

Specific examples of groups represented by General Formulae (a2-r-1) to (a2-r-7) are shown below.

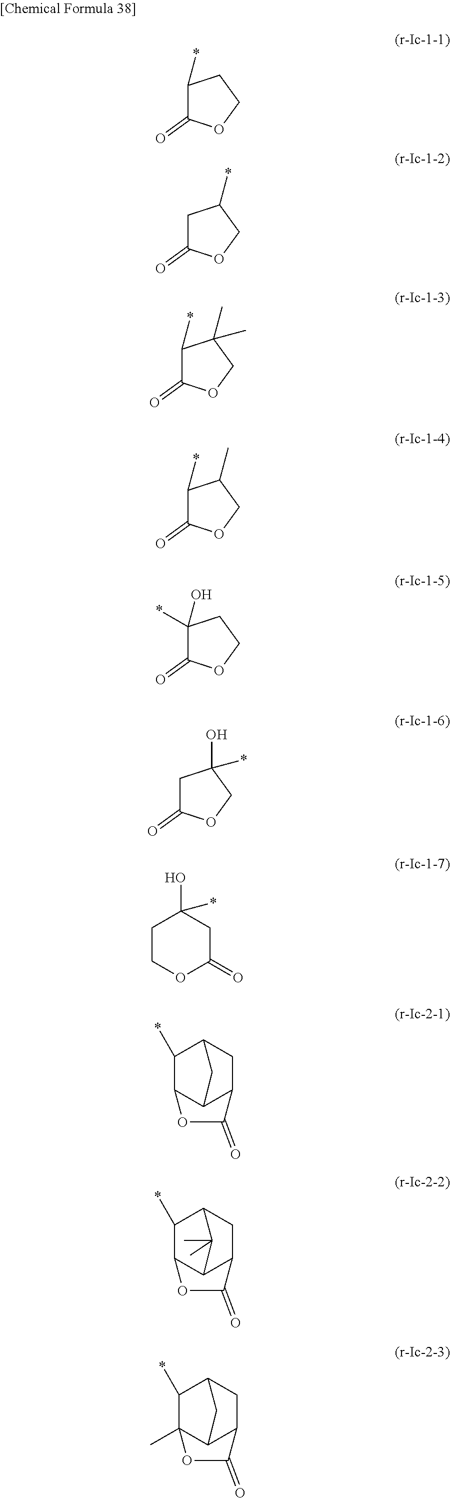

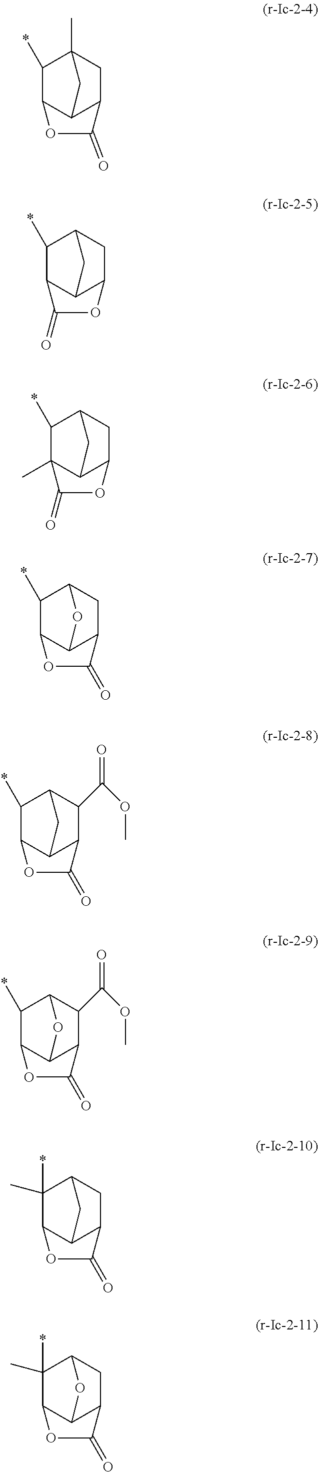

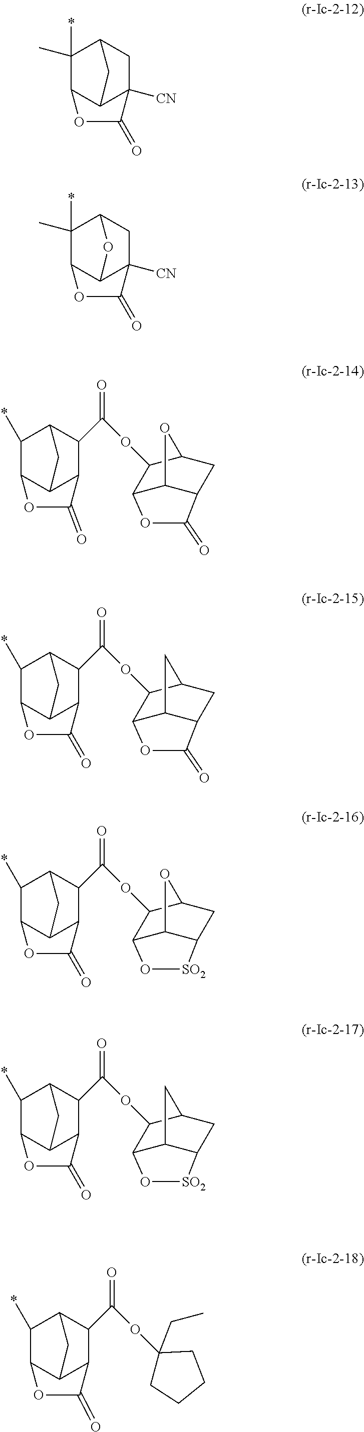

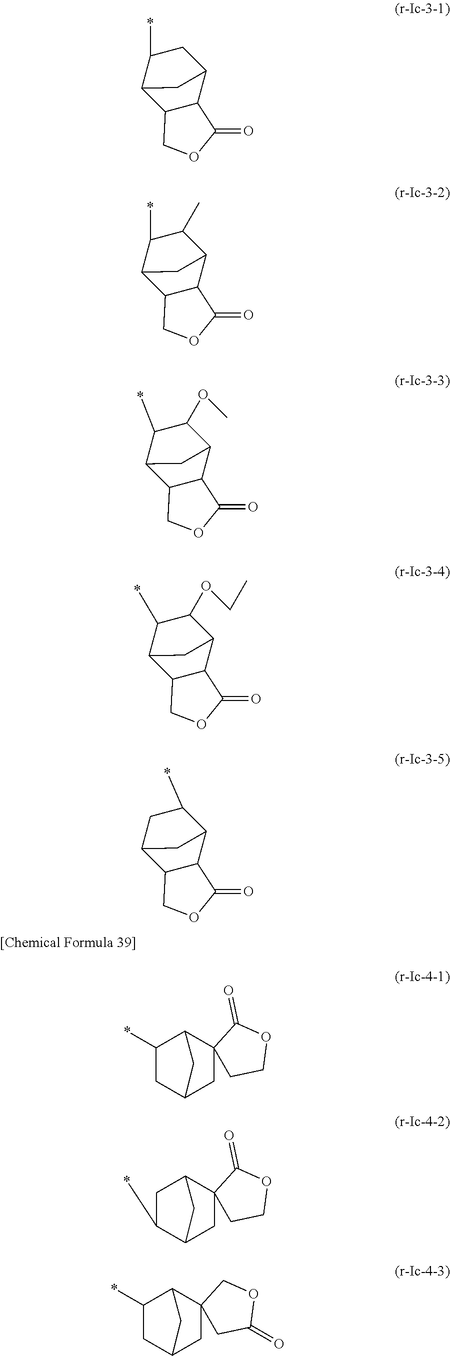

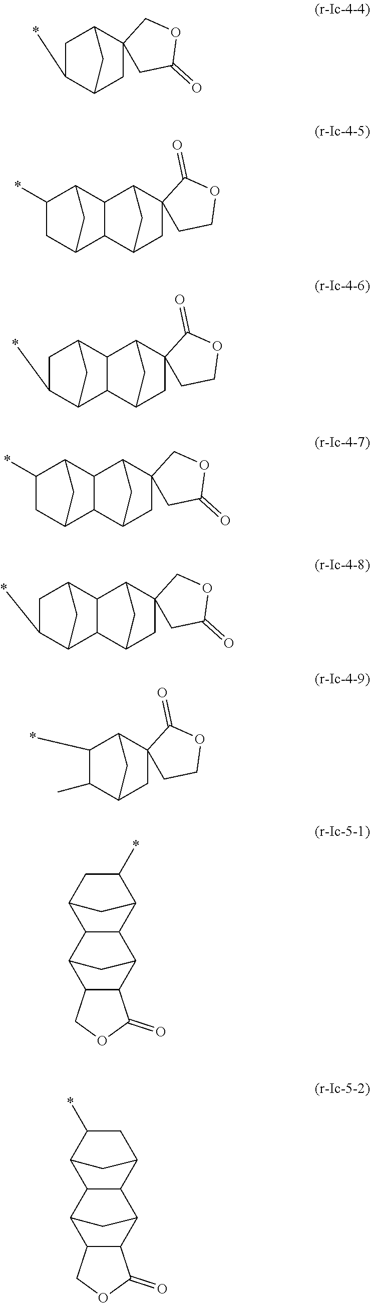

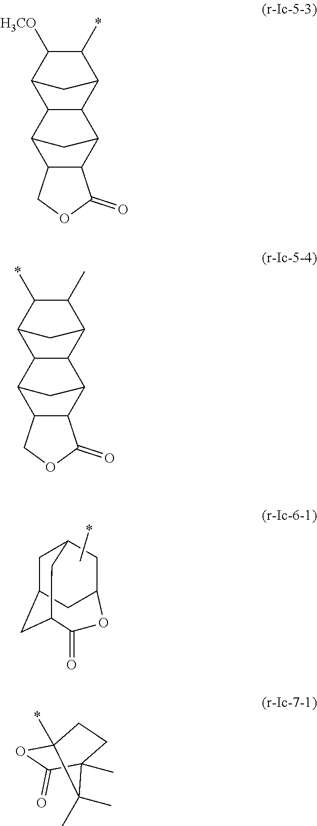

Among these, the structural unit (a2) is preferably a structural unit derived from an acrylic acid ester in which the hydrogen atom bonded to the carbon atom at the α-position may be substituted with a substituent.

Such a structural unit (a2) is preferably a structural unit represented by the following General Formula (a2-1).

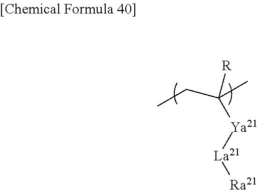

[in the formula, R is a hydrogen atom, an alkyl group having 1 to 5 carbon atoms or a halogenated alkyl group having 1 to 5 carbon atoms, Ya²¹ is a single bond or a divalent linking group, La²¹ is —O—, —COO—, —CON(R′)—, —OCO—, —CONHCO— or —CONHCS—, R′ is a hydrogen atom or a methyl group, when La²¹ is —O—, Ya²¹ does not become —CO—, and Ra²¹ is a lactone-containing cyclic group].

In Formula (a2-1), R is the same as defined above. R is preferably a hydrogen atom, an alkyl group having 1 to 5 carbon atoms or a fluorinated alkyl group having 1 to 5 carbon atoms, and in terms of industrial availability, a hydrogen atom or a methyl group is particularly preferable.

In Formula (a2-1), the divalent linking group for Ya²¹ is not particularly limited, and preferable examples thereof include a divalent hydrocarbon group which may have a substituent and a heteroatom-containing divalent linking group. Examples of divalent linking groups for Ya²¹ include the same divalent linking groups as for Ya^{x1} in General Formula (a10-1).

Ya²¹ is preferably a single bond, an ester bond [—C(═O)—O—], an ether bond (—O—), a linear or branched alkylene group, or a combination thereof.

In Formula (a2-1), preferably, Ya²¹ is a single bond, and La²′ is —COO— or —OCO—.

In Formula (a2-1), Ra²¹ is a lactone-containing cyclic group.

Preferable examples of lactone-containing cyclic groups for Ra²¹ include the groups represented by General Formulae (a2-r-1) to (a2-r-7).

The structural unit (a2) of the component (A1) may be of one type or of two or more types. The component (A1) may or may not have the structural unit (a2).

When the component (A1) has the structural unit (a2), the proportion of the structural unit (a2) based on a total amount (100 mol %) of all structural units constituting the component (A1) is preferably 1 to 20 mol %, more preferably 1 to 15 mol %, and still more preferably 1 to 10 mol %.

When the proportion of the structural unit (a2) is set to be equal to or larger than the preferable lower limit value, according to the above effects, the effects obtained when the structural unit (a2) is provided are sufficiently obtained, and when the proportion is set to be equal to or smaller than the upper limit value, a balance with other structural units can be achieved and various lithography properties become favorable.

### Structural Unit (a5):

The component (A1) may or may not have a structural unit (a5) that generates an acid upon exposure. As the structural unit (a5), known structural units can be used. When the structural unit (a5) is provided, the acid generated upon exposure is likely to be uniformly distributed within the resist film.

Preferable examples of structural units (a5) include a structural unit represented by the following General Formula (a5-1).

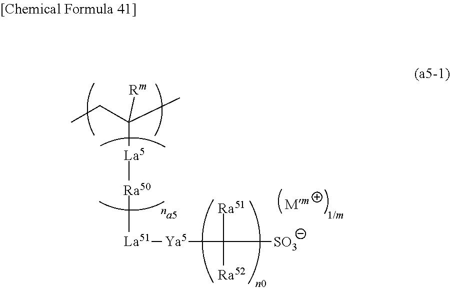

[in the formula, R^{m} is an alkyl group having 1 to 5 carbon atoms, a halogenated alkyl group having 1 to 5 carbon atoms, a halogen atom or a hydrogen atom, La5 is a divalent linking group or a single bond, Ra⁵⁰ is a divalent hydrocarbon group which may have a substituent, nₐ₅ is an integer of 0 to 2, La⁵¹ is a divalent linking group, Ya⁵ is a divalent linking group which may have a heteroatom or a single bond, Ra⁵¹ and Ra⁵² are each independently a hydrogen atom, a fluorine atom or a fluorinated alkyl group, n0 is an integer of 1 to 4, m is an integer of 1 or more, and M′^{m+} is an m-valent onium cation].

### {Anion Moiety}

In Formula (a5-1), R^{m} is an alkyl group having 1 to 5 carbon atoms, a halogenated alkyl group having 1 to 5 carbon atoms, a halogen atom or a hydrogen atom.

The alkyl group having 1 to 5 carbon atoms for R^{m} is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which some or all of the hydrogen atoms in the alkyl group having 1 to 5 carbon atoms are substituted with halogen atoms.

Examples of halogen atoms include a fluorine atom, a chlorine atom, a bromine atom, and an iodine atom. The halogen atom in the halogenated alkyl group is particularly preferably a fluorine atom.

R^{m} is preferably a hydrogen atom, an alkyl group having 1 to 5 carbon atoms or a fluorinated alkyl group having 1 to 5 carbon atoms, and in terms of industrial availability, a hydrogen atom or a methyl group is most preferable.

In Formula (a5-1), La5 is a divalent linking group or a single bond.

The divalent linking group for La5 is not particularly limited, and preferable examples thereof include a divalent hydrocarbon group which may have a substituent and a heteroatom-containing divalent linking group, which are the same as the divalent hydrocarbon group which may have a substituent and the heteroatom-containing divalent linking group exemplified as the divalent linking group for Ya^{x1}.

Among the above examples, La5 is preferably an ester bond [—C(═O)—O—, —O—C(═O)—], an ether bond (—O—), a linear or branched alkylene group, an aromatic hydrocarbon group or a combination thereof, or a single bond. Among the above examples, La5 is more preferably an ester bond [—C(═O)—O—, —O—C(═O)—] or a single bond, and still more preferably an ester bond [—C(═O)—O—, —O—C(═O)—].

In Formula (a5-1), Ra⁵⁰ is a divalent hydrocarbon group which may have a substituent.

The divalent hydrocarbon group for Ra⁵⁰ may be an aliphatic hydrocarbon group or an aromatic hydrocarbon group.

### Aliphatic Hydrocarbon Group for Ra⁵⁰

The aliphatic hydrocarbon group is a hydrocarbon group having no aromaticity. The aliphatic hydrocarbon group may be saturated or unsaturated, and is generally preferably saturated.

Examples of aliphatic hydrocarbon groups include a linear or branched aliphatic hydrocarbon group and an aliphatic hydrocarbon group containing a ring in the structure.

### Linear or Branched Aliphatic Hydrocarbon Group

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The linear or branched aliphatic hydrocarbon group may or may not have a substituent. Examples of substituents include a fluorine atom, a fluorinated alkyl group having 1 to 5 carbon atoms and substituted with a fluorine atom, and a carbonyl group.

### Aliphatic Hydrocarbon Group Containing Ring in Structure

Examples of aliphatic hydrocarbon groups containing a ring in the structure include a cyclic aliphatic hydrocarbon group which may contain a heteroatom-containing substituent in the ring structure (a group in which two hydrogen atoms have been removed from an aliphatic hydrocarbon ring), a group in which the cyclic aliphatic hydrocarbon group is bonded to the end of a linear or branched aliphatic hydrocarbon group, and a group in which the cyclic aliphatic hydrocarbon group is inserted into a linear or branched aliphatic hydrocarbon group. Examples of linear or branched aliphatic hydrocarbon groups include the same groups described above.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic alicyclic hydrocarbon group is preferably a group in which two hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms have been removed from a polycycloalkane, the polycycloalkane is preferably one having 7 to 12 carbon atoms, and specific examples thereof include adamantane, norbornane, isobornane, tricyclo[5.2.1.0^{2,6}]decane, and tetracyclododecane.

The cyclic aliphatic hydrocarbon group may or may not have a substituent. Examples of substituents include an alkyl group, an alkoxy group, a halogen atom, a halogenated alkyl group, a hydroxy group, and a carbonyl group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as a substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of halogen atoms as the substituent include a fluorine atom, a chlorine atom, a bromine atom, and an iodine atom, and a fluorine atom is preferable.

Examples of halogenated alkyl groups as the substituent include a group in which some or all of the hydrogen atoms in the alkyl group are substituted with a halogen atom.

In the cyclic aliphatic hydrocarbon group, some carbon atoms constituting the ring structure may be substituted with a heteroatom-containing substituent. The heteroatom-containing substituent is preferably —O—, —C(═O)—O—, —S—, —S(═O)₂—, or —S(═O)₂—O—.

### Aromatic Hydrocarbon Group for Ra⁵⁰

The aromatic hydrocarbon group is a hydrocarbon group having at least one aromatic ring.

The aromatic ring is not particularly limited as long as it is a cyclic conjugated system having 4n+2 π electrons, and may be monocyclic or polycyclic. The aromatic ring preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms does not include the number of carbon atoms in the substituent. Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

Specific examples of aromatic hydrocarbon groups include a group in which two hydrogen atoms have been removed from the aromatic hydrocarbon ring or aromatic heterocycle (an arylene group or a heteroarylene group); a group in which two hydrogen atoms have been removed from an aromatic compound containing two or more aromatic rings (for example, biphenyl, fluorene, etc.); and a group in which one hydrogen atom of a group (an aryl group or a heteroaryl group) in which one hydrogen atom has been removed from the aromatic hydrocarbon ring or aromatic heterocycle is substituted with an alkylene group (for example, a group in which one hydrogen atom has been additionally removed from the aryl group in the arylalkyl group such as a benzyl group, a phenethyl group, a 1-naphthyl methyl group, a 2-naphthyl methyl group, a 1-naphthyl ethyl group, and a 2-naphthyl ethyl group). The alkylene group bonded to the aryl group or heteroaryl group preferably has 1 to 4 carbon atoms, more preferably has 1 or 2 carbon atoms, and particularly preferably has 1 carbon atom.

In the aromatic hydrocarbon group, the hydrogen atom in the aromatic hydrocarbon group may be substituted with a substituent. For example, the hydrogen atom in the aromatic hydrocarbon group, which is bonded to an aromatic ring, may be substituted with a substituent. Examples of substituents include an alkyl group, an alkoxy group, a halogen atom, a halogenated alkyl group, and a hydroxy group.

The alkyl group as the substituent is preferably an alkyl group having 1 to 5 carbon atoms, and most preferably a methyl group, an ethyl group, a propyl group, an n-butyl group, or a tert-butyl group.

Examples of alkoxy groups, halogen atoms and halogenated alkyl groups as the substituent include those exemplified as the substituent that substitutes a hydrogen atom in the cyclic aliphatic hydrocarbon group.

In Formula (a5-1), nₐ₅ is an integer of 0 to 2.

Among the above examples, Ra⁵⁰ is preferably an aliphatic hydrocarbon group containing a ring in the structure, more preferably a cyclic aliphatic hydrocarbon group which may contain a heteroatom-containing substituent in the ring structure, and still more preferably an alicyclic hydrocarbon group which is a polycyclic group or a monocyclic group and which may have a substituent.

Alternatively, among the above examples, Ra⁵⁰ is preferably an aromatic hydrocarbon group.

When nₐ₅ is 2, two Ra⁵⁰'s may each be an alicyclic hydrocarbon group which may have a substituent, each may be an aromatic hydrocarbon group, or they may be a combination of an alicyclic hydrocarbon group which may have a substituent and an aromatic hydrocarbon group.

In Formula (a5-1), La⁵¹ is a divalent linking group.

Examples of divalent linking groups for La⁵¹ include non-hydrocarbon-based oxygen atom-containing linking groups such as an oxygen atom (ether bond: —O—), an ester bond (—C(═O)—O—), an oxycarbonyl group (—O—C(═O)—), an amide bond (—C(═O)—NH—), a carbonyl group (—C(═O)—), and a carbonate bond (—O—C(═O)—O—); and a combination of a non-hydrocarbon-based oxygen atom-containing linking group and an alkylene group. A sulfonyl group (—SO₂—) may be additionally linked to this combination.

Examples of such divalent linking groups include linking groups represented by the following General Formulae (L-a1-1) to (L-a1-8). Here, in the following General Formulae (L-a1-1) to (L-a1-8), one bonded to Ra⁵⁰ in Formula (a5-1) is V′¹⁰¹ in the following General Formulae (L-al-1) to (L-al-8).

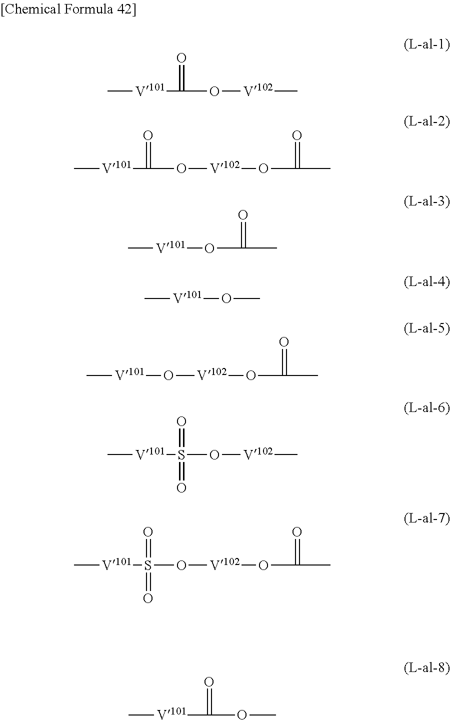

[in the formula, V′¹⁰¹ is a single bond or an alkylene group having 1 to 5 carbon atoms, and V′¹⁰² is a divalent saturated hydrocarbon group having 1 to 30 carbon atoms].

The divalent saturated hydrocarbon group for V′¹⁰² is preferably an alkylene group having 1 to 30 carbon atoms, more preferably an alkylene group having 1 to 10 carbon atoms, and still more preferably an alkylene group having 1 to 5 carbon atoms.

The alkylene group for V′¹⁰¹ and V′¹⁰² may be a linear alkylene group or a branched alkylene group, and is preferably a linear alkylene group.

Specific examples of alkylene groups for V′¹⁰¹ and V′¹⁰² include a methylene group [—CH₂—]; alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; an ethylene group [—CH₂CH₂—]; alkylethylene groups such as —CH(CH₃)CH₂—, —CH(CH₃)CH(CH₃)—, —C(CH₃)₂CH₂—, and —CH(CH₂CH₃)CH₂—; a trimethylene group (n-propylene group) [—CH₂CH₂CH₂—]; alkyltrimethylene groups such as —CH(CH₃)CH₂CH₂— and —CH₂CH(CH₃)CH₂—; a tetramethylene group [—CH₂CH₂CH₂CH₂—]; alkyltetramethylene groups such as —CH(CH₃)CH₂CH₂CH₂— and —CH₂CH(CH₃)CH₂CH₂—; and a pentamethylene group [—CH₂CH₂CH₂CH₂CH₂—].

In addition, some methylene groups in the alkylene group for V′¹⁰¹ or V′¹⁰² may be substituted with a divalent aliphatic cyclic group having 5 to 10 carbon atoms. The aliphatic cyclic group is preferably a divalent group in which one hydrogen atom has been additionally removed from a cyclic aliphatic hydrocarbon group (a monocyclic aliphatic hydrocarbon group, and a polycyclic aliphatic hydrocarbon group) for Ra′³ in Formula (a1-r-1), and more preferably a cyclohexylene group, a 1,5-adamantylene group or a 2,6-adamantylene group.

La⁵¹ is preferably a divalent linking group containing an ester bond or a divalent linking group containing an ether bond, more preferably a linking group represented by each of Formulae (L-a1-1) to (L-a1-5), and (L-al-8), and still more preferably a linking group represented by (L-al-3) or (L-al-8).

In Formula (a5-1), Ya⁵ is a divalent linking group which may have a heteroatom or a single bond.

The divalent linking group for Ya⁵ is not particularly limited, and preferable examples thereof include a divalent hydrocarbon group which may have a substituent and a heteroatom-containing divalent linking group.

The divalent hydrocarbon group which may have a substituent and the heteroatom-containing divalent linking group for Ya⁵ are the same as the divalent hydrocarbon group which may have a substituent and the heteroatom-containing divalent linking group exemplified as the divalent linking group for Ya

Among the above examples, Ya⁵ is preferably a linear or branched alkylene group or a single bond and more preferably a single bond.

In Formula (a5-1), Ra⁵¹ and Ra⁵² are each independently a hydrogen atom, a fluorine atom or a fluorinated alkyl group.

The fluorinated alkyl groups for Ra⁵¹ and Ra⁵² are each preferably a linear or branched fluorinated alkyl group having 1 to 5 carbon atoms and more preferably a trifluoromethyl group.

In Formula (a5-1), it is preferable that at least one of Ra⁵¹ and Ra⁵² bonded to the carbon atom adjacent to SO₃⁻ be a fluorine atom in consideration of acid strength.

In Formula (a5-1), n0 is an integer of 1 to 4, and preferably 1, 2 or 3.

### {Cation Moiety}

In Formula (a5-1), M′^{m+} is an m-valent onium cation. Among these, M′^{m+} is preferably a sulfonium cation or an iodonium cation. m is an integer of 1 or more.

Examples of preferable cation moieties ((M′^{m+})_{1/m}) include organic cations represented by the following General Formulae (ca-1) to (ca-3).

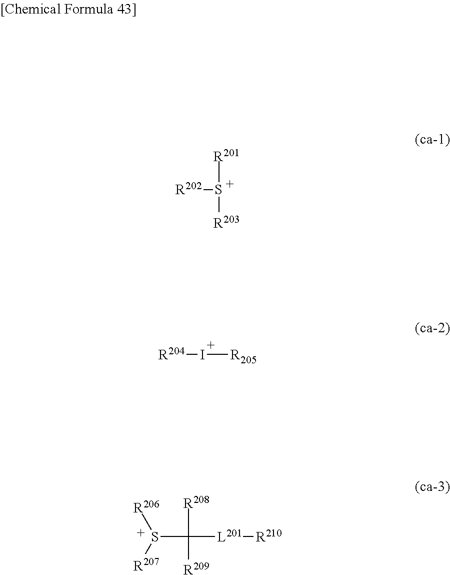

[in the formula, R²⁰¹ to R²⁰⁷ are each independently an aryl group which may have a substituent, an alkyl group which may have a substituent or an alkenyl group which may have a substituent, R²⁰¹ to R²⁰³ and R²⁰⁶ and R²⁰⁷ may be bonded to each other to form a ring together with a sulfur atom in the formula, R²⁰⁸ and R²⁰⁹ are each independently a hydrogen atom or an alkyl group having 1 to 5 carbon atoms, R²¹⁰ is an aryl group which may have a substituent, an alkyl group which may have a substituent, an alkenyl group which may have a substituent, or a —SO₂-containing cyclic group which may have a substituent, and L²⁰¹ is —C(═O)— or —C(═O)—O—].

In General Formulae (ca-1) to (ca-3), examples of aryl groups for R²⁰¹ to R²⁰⁷ include an unsubstituted aryl group having 6 to 20 carbon atoms, and a phenyl group and a naphthyl group are preferable.

The alkyl group for R²⁰¹ to R²⁰⁷ is a chain or cyclic alkyl group and preferably has 1 to 30 carbon atoms.

The alkenyl group for R²⁰¹ to R²⁰⁷ preferably has 2 to 10 carbon atoms.

Examples of substituents that R²⁰¹ to R²⁰⁷, and R²¹⁰ may have include an alkyl group, a halogen atom, a halogenated alkyl group, a carbonyl group, a cyano group, an amino group, an aryl group, and groups represented by the following General Formulae (ca-r-1) to (ca-r-7).

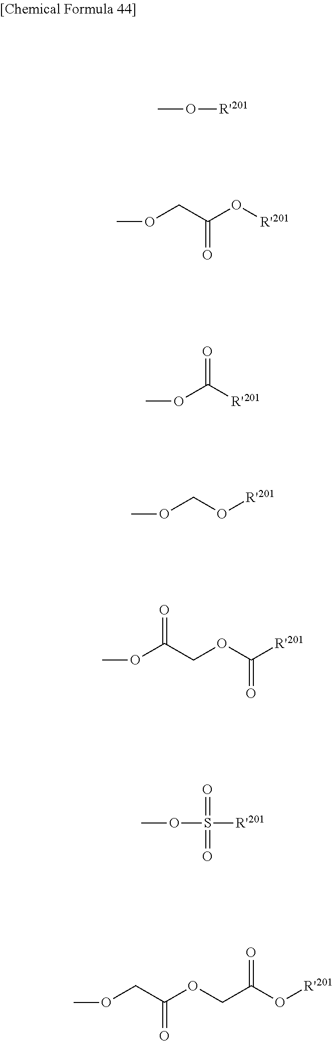

[in the formula, R′²⁰¹'s are each independently a hydrogen atom, a cyclic group which may have a substituent, chain alkyl group which may have a substituent, or a chain alkenyl group which may have a substituent].

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group having no aromaticity. In addition, the aliphatic hydrocarbon group may be saturated or unsaturated, and is generally preferably saturated.

The aromatic hydrocarbon group for R′²⁰¹ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms does not include the number of carbon atoms in the substituent.

Specific examples of aromatic rings in the aromatic hydrocarbon group for R′²⁰¹ include benzene, fluorene, naphthalene, anthracene, phenanthrene, biphenyl and aromatic heterocycles in which some carbon atoms constituting these aromatic rings are substituted with heteroatoms. Examples of heteroatoms in aromatic heterocycles include an oxygen atom, a sulfur atom, and a nitrogen atom.

Specific examples of aromatic hydrocarbon groups for R′²⁰¹ include a group in which one hydrogen atom has been removed from the aromatic ring (an aryl group: for example, a phenyl group, a naphthyl group, etc.), and a group in which one hydrogen atom in the aromatic ring is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group). The alkylene group (the alkyl chain in the arylalkyl group) preferably has 1 to 4 carbon atoms, more preferably has 1 or 2 carbon atoms, and particularly preferably has 1 carbon atom.

Examples of cyclic aliphatic hydrocarbon groups for R′²⁰¹ include an aliphatic hydrocarbon group containing a ring in the structure.

Examples of aliphatic hydrocarbon groups containing a ring in the structure include an alicyclic hydrocarbon group (a group in which one hydrogen atom has been removed from an aliphatic hydrocarbon ring), a group in which an alicyclic hydrocarbon group is bonded to the end of a linear or branched aliphatic hydrocarbon group, and a group in which an alicyclic hydrocarbon group is inserted into a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group in which one or more hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which one or more hydrogen atoms have been removed from a polycycloalkane, and the polycycloalkane is preferably one having 7 to 30 carbon atoms. Among these, the polycycloalkane is more preferably a polycycloalkane having a crosslinked ring type polycyclic framework such as adamantane, norbornane, isobornane, tricyclo[5.2.1.02, 6]decane, and tetracyclododecane; or a polycycloalkane having a fused ring type polycyclic framework such as a cyclic group having a steroid framework.

Among these, the cyclic aliphatic hydrocarbon group for R′²⁰¹ is preferably a group in which one or more hydrogen atoms have been removed from a monocycloalkane or polycycloalkane, more preferably a group in which one hydrogen atom has been removed from a polycycloalkane, particularly preferably an adamantyl group or a norbornyl group, and most preferably an adamantyl group.

The linear or branched aliphatic hydrocarbon group which may be bonded to an alicyclic hydrocarbon group has preferably 1 to 10 carbon atoms, more preferably has 1 to 6 carbon atoms, still more preferably has 1 to 4 carbon atoms, and particularly preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group for R′²⁰¹ may contain a heteroatom such as in a heterocycle. Specific examples thereof include lactone-containing cyclic groups represented by General Formulae (a2-r-1) to (a2-r-7), —SO₂-containing cyclic groups represented by the following General Formulae (b5-r-1) to (b5-r-4), and other heterocyclic groups represented by the following Chemical Formulae (r-hr-1) to (r-hr-16).

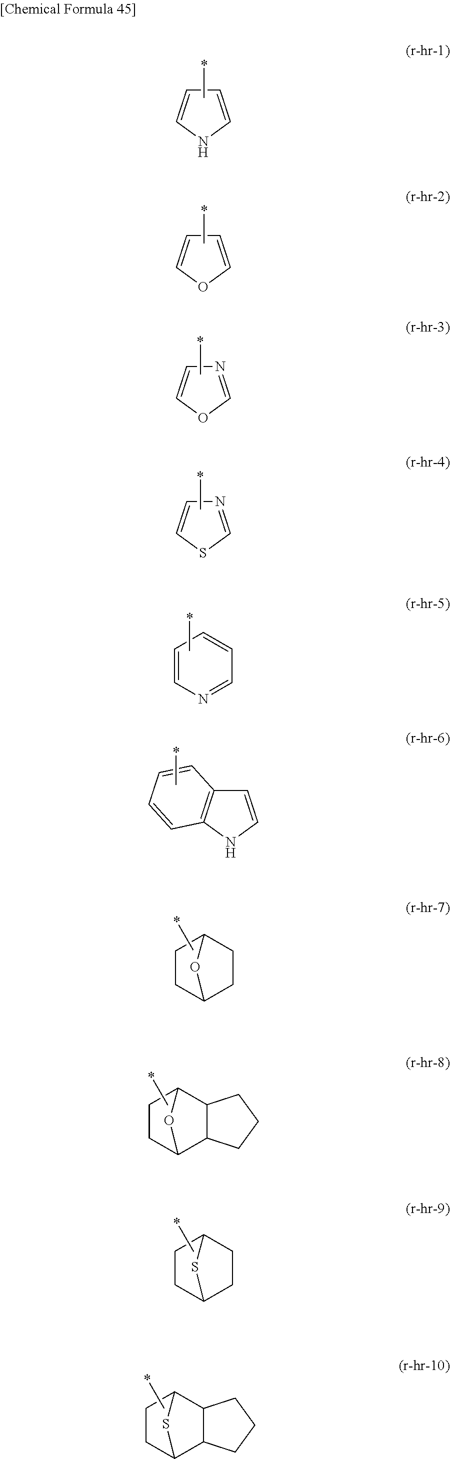

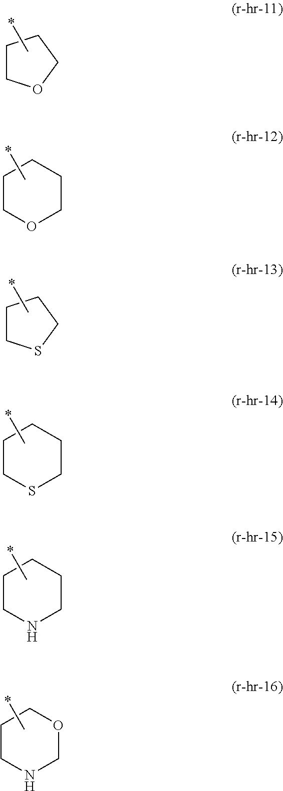

Examples of substituents in the cyclic group for R′²⁰¹ include an alkyl group, an alkoxy group, a halogen atom, a halogenated alkyl group, a hydroxy group, a carbonyl group, and a nitro group.

The alkyl group as a substituent is preferably an alkyl group having 1 to 5 carbon atoms, and most preferably a methyl group, an ethyl group, a propyl group, an n-butyl group, or a tert-butyl group.

The alkoxy group as a substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as the substituent is preferably a fluorine atom.

Examples of halogenated alkyl groups as a substituent include an alkyl group having 1 to 5 carbon atoms, for example, a group in which some or all of the hydrogen atoms are substituted with halogen atoms such as a methyl group, an ethyl group, a propyl group, an n-butyl group, and a tert-butyl group.

The carbonyl group as a substituent is a group that substitutes a methylene group (—CH₂—) constituting a cyclic hydrocarbon group.

Chain alkyl group which may have substituent:

The chain alkyl group for R′²⁰¹ may be either linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain alkenyl group which may have substituent:

The chain alkenyl group for R′²⁰¹ may be linear or branched, and preferably has 2 to 10 carbon atoms, more preferably has 2 to 5 carbon atoms, still more preferably has 2 to 4 carbon atoms, and particularly preferably has 3 carbon atoms. Examples of linear alkenyl groups include a vinyl group, a propenyl group (allyl group), and a butenyl group. Examples of branched alkenyl groups include a 1-methylvinyl group, a 2-methylvinyl group, a 1-methylpropenyl group, and a 2-methylpropenyl group.

Among the above examples, the chain alkenyl group is preferably a linear alkenyl group, more preferably a vinyl group or a propenyl group, and particularly preferably a vinyl group.

Examples of substituents in the chain alkyl group or alkenyl group for R′²⁰¹ include an alkoxy group, a halogen atom, a halogenated alkyl group, a hydroxy group, a carbonyl group, a nitro group, an amino group, and the cyclic groups for R′²⁰¹.

Examples of cyclic groups which may have a substituent, chain alkyl groups which may have a substituent, or chain alkenyl groups which may have a substituent for R′²⁰¹ include, in addition to the above examples, the same groups as the acid-dissociable group represented by Formula (al-r-2) as a cyclic group which may have a substituent or a chain alkyl group which may have a substituent.

Among these, R′²⁰¹ is preferably a cyclic group which may have a substituent and more preferably a cyclic hydrocarbon group which may have a substituent. More specifically, for example, a phenyl group, a naphthyl group, and a group in which one or more hydrogen atoms have been removed from a polycycloalkane; lactone-containing cyclic groups represented by General Formulae (a2-r-1) to (a2-r-7); and —SO₂— containing cyclic groups represented by the following General Formulae (b5-r-1) to (b5-r-4) are preferable.

In General Formulae (ca-1) to (ca-3), when R²⁰¹ to R²⁰³ and R²⁰⁶ and R²⁰⁷ are bonded to each other to form a ring together with a sulfur atom in the formula, they may be bonded via a heteroatom such as a sulfur atom, an oxygen atom, or a nitrogen atom, or a functional group such as a carbonyl group, —SO—, —SO₂—, —SO₃—, —COO—, —CONH— or —N(R_{N})—(R_{N} is an alkyl group having 1 to 5 carbon atoms). As the ring to be formed, one ring containing a sulfur atom in its ring framework in the formula is preferably a 3- to 10-membered ring and particularly preferably a 5- to 7-membered ring, including the sulfur atom. Specific examples of rings formed include a thiophene ring, a thiazole ring, a benzothiophene ring, a dibenzothiophene ring, a 9H-thioxanthene ring, a thioxanthone ring, a thianthrene ring, a phenoxathiin ring, a tetrahydrothiophenium ring, and a tetrahydrothiopyranium ring.

R²⁰⁸ and R²⁰⁹ are each independently a hydrogen atom or an alkyl group having 1 to 5 carbon atoms, and preferably a hydrogen atom or an alkyl group having 1 to 3 carbon atoms, and when they are an alkyl group, they may be bonded to each other to form a ring.

R²¹⁰ is an aryl group which may have a substituent, an alkyl group which may have a substituent, an alkenyl group which may have a substituent, or a —SO₂-containing cyclic group which may have a substituent.

Examples of aryl groups for R²¹⁰ include an unsubstituted aryl group having 6 to 20 carbon atoms, and a phenyl group and a naphthyl group are preferable.

The alkyl group for R²¹⁰ is preferably a chain or cyclic alkyl group having 1 to 30 carbon atoms.

The alkenyl group for R²¹⁰ preferably has 2 to 10 carbon atoms.

The —SO₂-containing cyclic group for R²¹⁰ is not particularly limited, and any —SO₂-containing cyclic group can be used. Specific examples thereof include groups represented by the following General Formulae (b5-r-1) to (b5-r-4), and a “—SO₂-containing polycyclic group” is preferable, and a group represented by General Formula (b5-r-1) is more preferable.

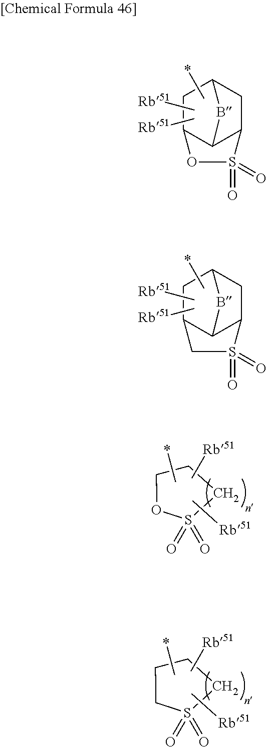

[in the formula, Rb′⁵¹'s are each independently a hydrogen atom, an alkyl group, an alkoxy group, a halogen atom, a halogenated alkyl group, a hydroxyl group, —COOR″, —OC(═O)R″, a hydroxyalkyl group or a cyano group; R″ is a hydrogen atom, an alkyl group, a lactone-containing cyclic group, or a —SO₂-containing cyclic group; B″ is an alkylene group having 1 to 5 carbon atoms which may contain an oxygen atom or a sulfur atom, an oxygen atom or a sulfur atom, n′ is an integer of 0 to 2, and indicates a bond].

In General Formulae (b5-r-1) and (b5-r-2), B″ is an alkylene group having 1 to 5 carbon atoms which may contain an oxygen atom or a sulfur atom, an oxygen atom or a sulfur atom.

B″ is preferably an alkylene group having 1 to 5 carbon atoms or —O—, more preferably an alkylene group having 1 to 5 carbon atoms, and still more preferably a methylene group.

In General Formulae (b5-r-1) to (b5-r-4), Rb′⁵¹'s are each independently a hydrogen atom, an alkyl group, an alkoxy group, a halogen atom, a halogenated alkyl group, a hydroxyl group, —COOR″, —OC(═O)R″, a hydroxyalkyl group or a cyano group, and among these, they are each independently preferably a hydrogen atom or a cyano group.

Specific examples of groups represented by General Formulae (b5-r-1) to (b5-r-4) are shown below. In the formulae, “Ac” is an acetyl group.

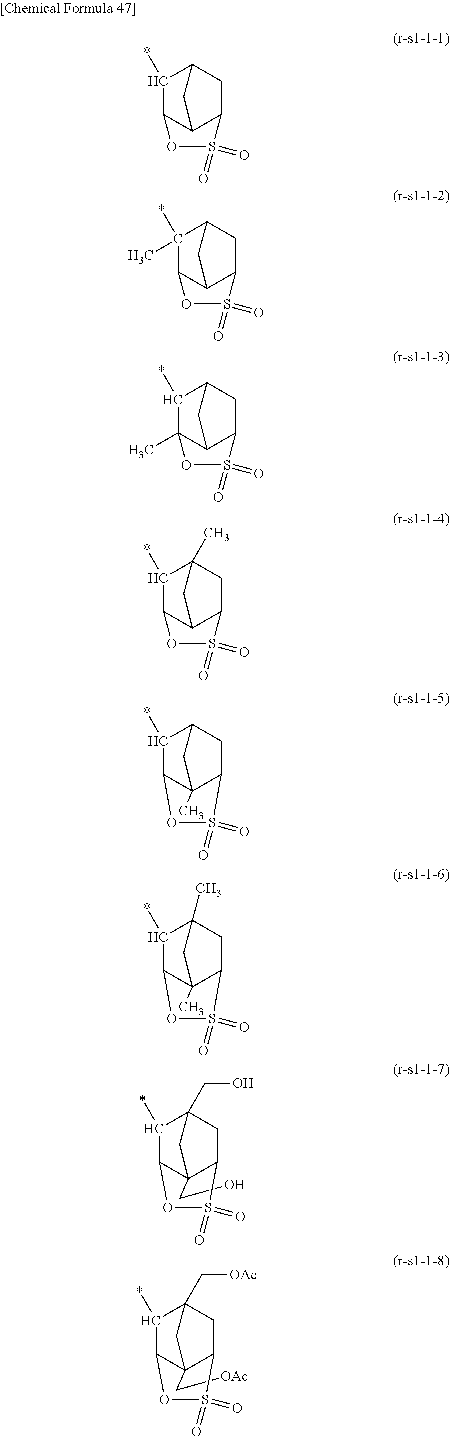

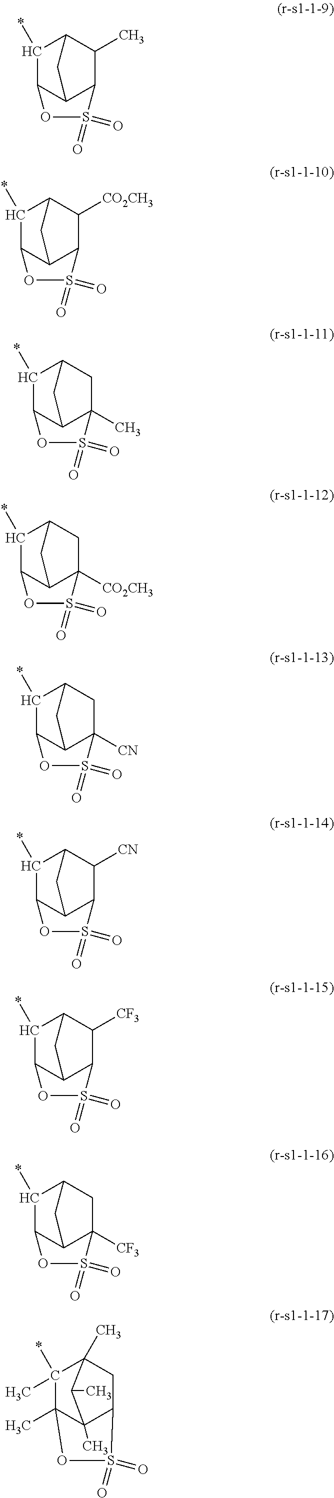

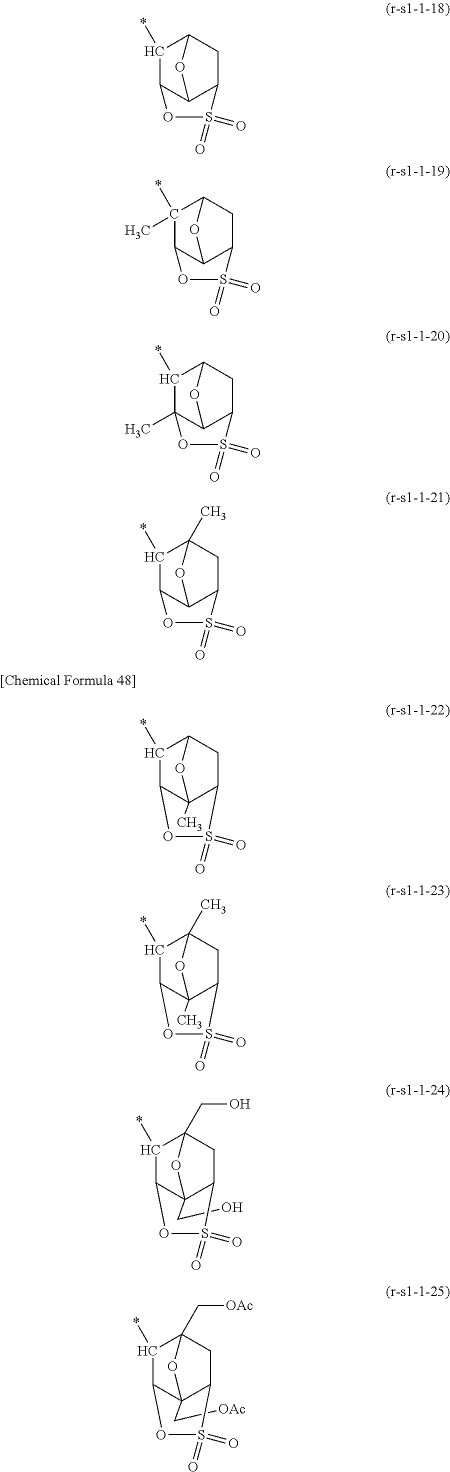

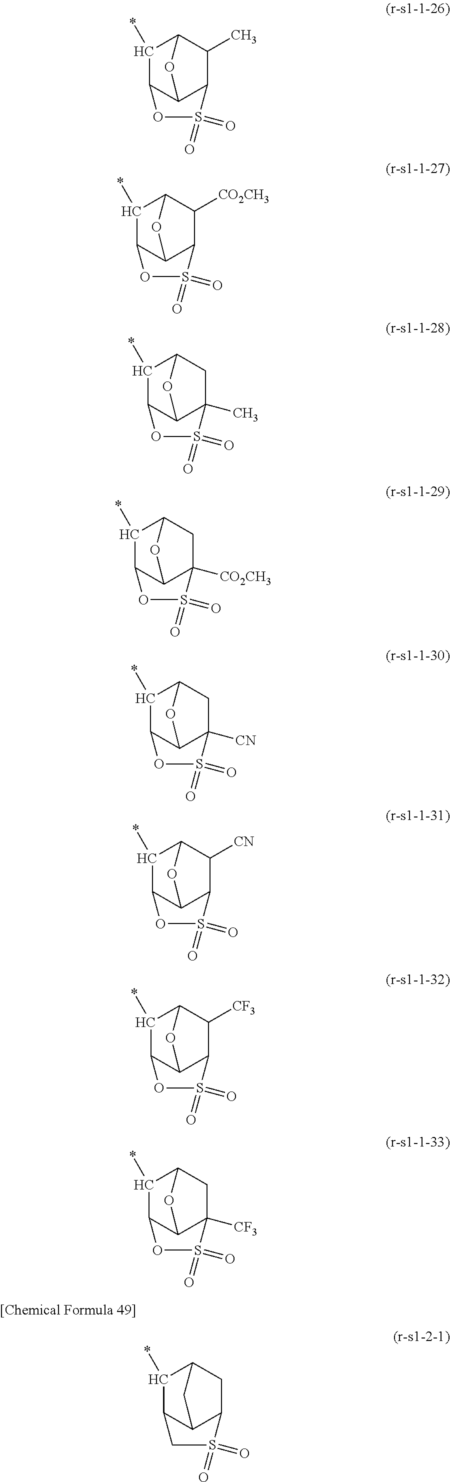

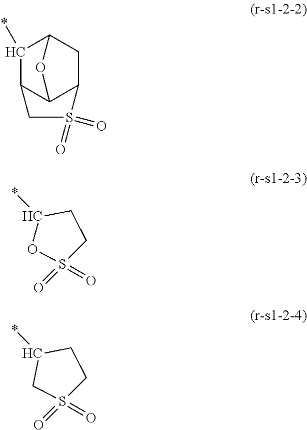

Specific examples of preferable cations represented by Formula (ca-1) include cations represented by the following chemical formulae.

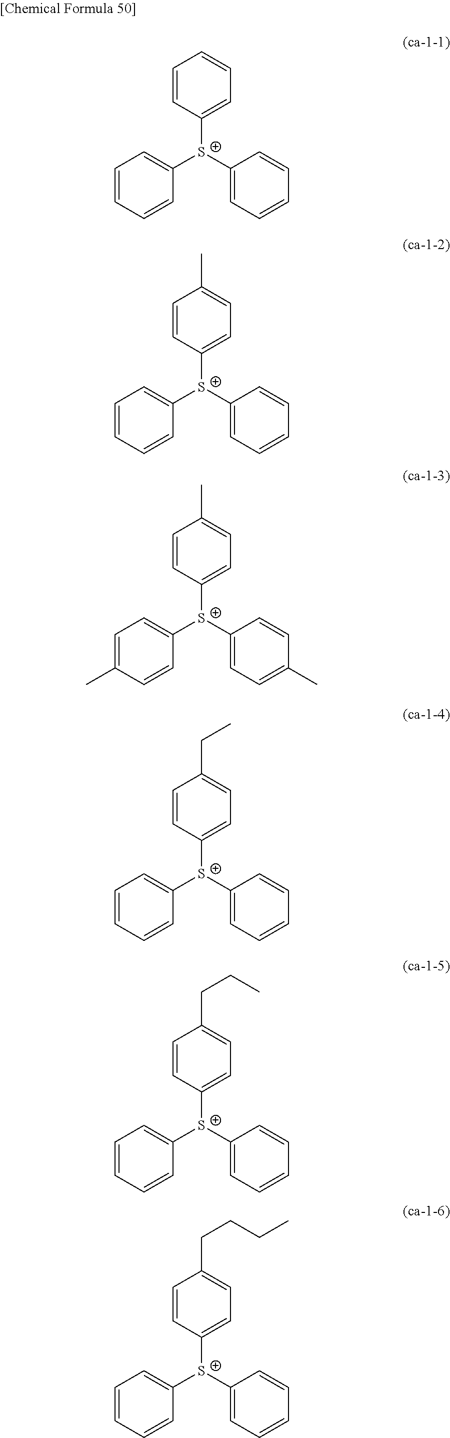

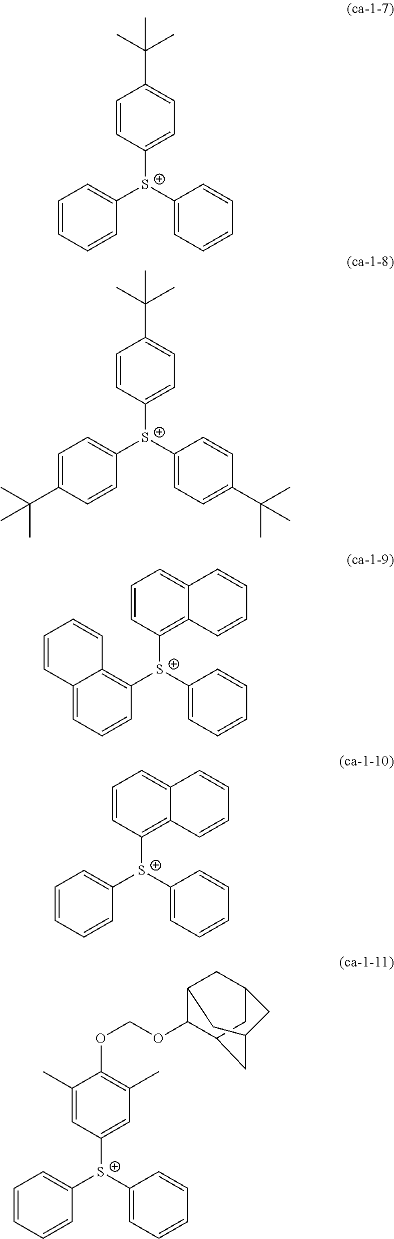 [SMILES: CC(C)(C)c1ccc([S+](c2ccccc2)c2ccccc2)cc1 ; CC(C)(C)c1ccc([S+](c2ccc(C(C)(C)C)cc2)c2ccc(C(C)(C)C)cc2)cc1 ; c1ccc([S+](c2cccc3ccccc23)c2cccc3ccccc23)cc1 ; c1ccc([S+](c2ccccc2)c2cccc3ccccc23)cc1 ; Cc1cc([S+](c2ccccc2)c2ccccc2)cc(C)c1OCOC1C2CC3CC(C2)CC1C3]

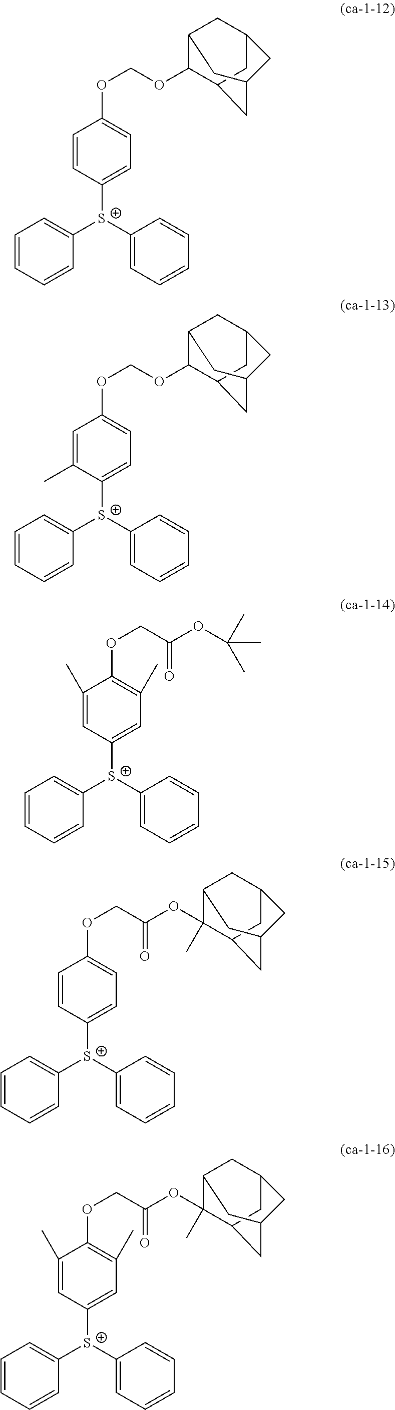 [SMILES: c1ccc([S+](c2ccccc2)c2ccc(OCOC3C4CC5CC(C4)CC3C5)cc2)cc1 ; Cc1cc(OCOC2C3CC4CC(C3)CC2C4)ccc1[S+](c1ccccc1)c1ccccc1 ; Cc1cc([S+](c2ccccc2)c2ccccc2)cc(C)c1OCC(=O)OC(C)(C)C ; CC1(OC(=O)COc2ccc([S+](c3ccccc3)c3ccccc3)cc2)C2CC3CC(C2)CC1C3 ; Cc1cc([S+](c2ccccc2)c2ccccc2)cc(C)c1OCC(=O)OC1(C)C2CC3CC(C2)CC1C3]

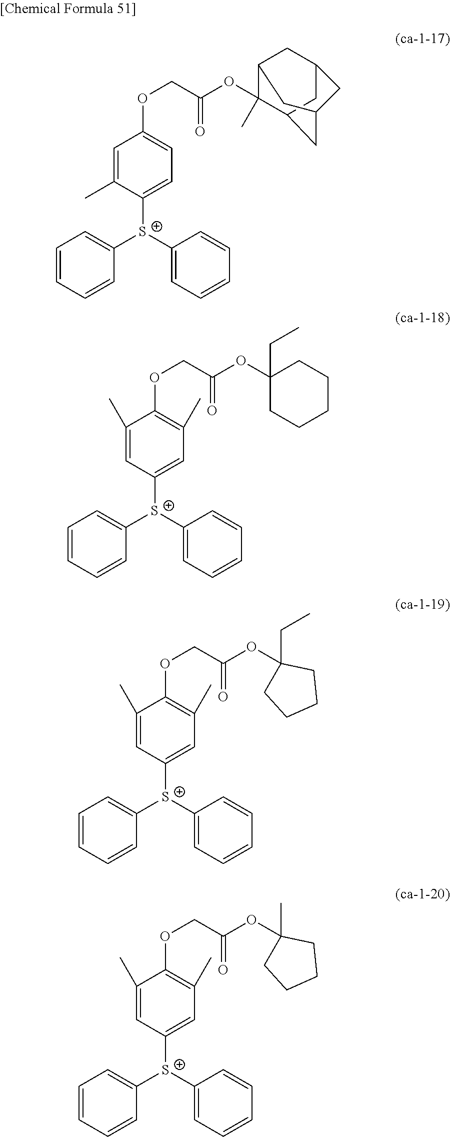 [SMILES: Cc1cc(OCC(=O)OC2(C)C3CC4CC(C3)CC2C4)ccc1[S+](c1ccccc1)c1ccccc1 ; CCC1(OC(=O)COc2c(C)cc([S+](c3ccccc3)c3ccccc3)cc2C)CCCCC1 ; CCC1(OC(=O)COc2c(C)cc([S+](c3ccccc3)c3ccccc3)cc2C)CCCC1 ; Cc1cc([S+](c2ccccc2)c2ccccc2)cc(C)c1OCC(=O)OC1(C)CCCC1]

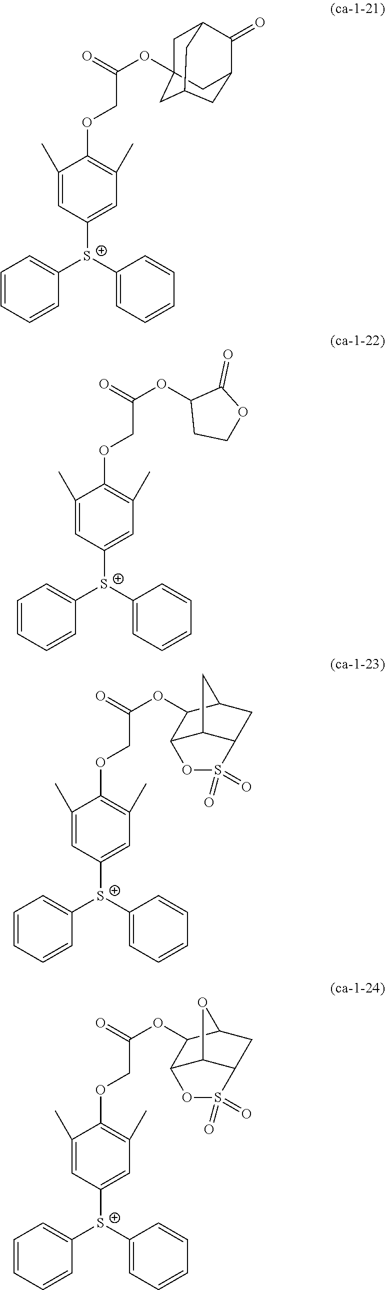 [SMILES: Cc1cc([S+](c2ccccc2)c2ccccc2)cc(C)c1OCC(=O)OC12CC3CC(C1)C(=O)C(C3)C2 ; Cc1cc([S+](c2ccccc2)c2ccccc2)cc(C)c1OCC(=O)OC1CCOC1=O ; Cc1cc([S+](c2ccccc2)c2ccccc2)cc(C)c1OCC(=O)OC1C2CC3C1OS(=O)(=O)C3C2 ; Cc1cc([S+](c2ccccc2)c2ccccc2)cc(C)c1OCC(=O)OC1C2CC3C(O2)C1OS3(=O)=O]

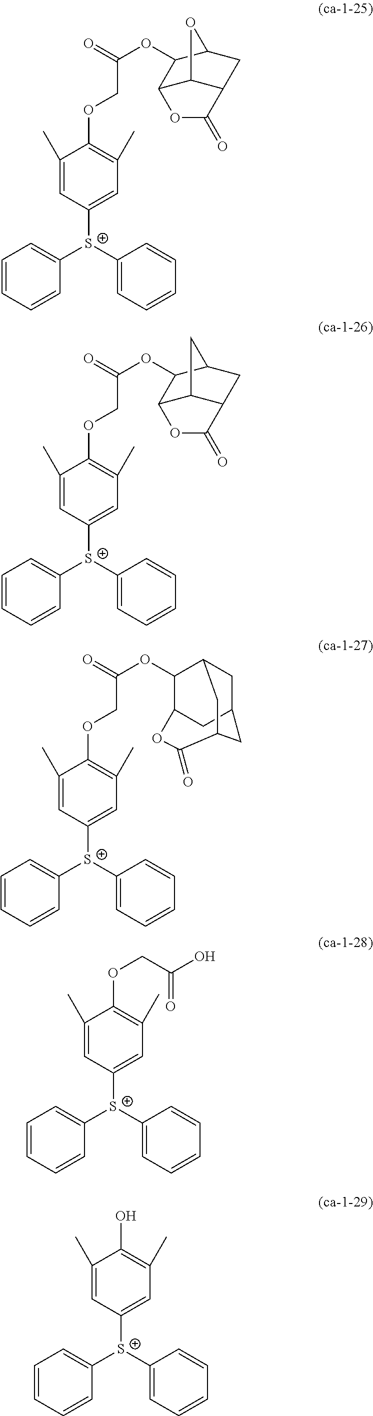 [SMILES: Cc1cc([S+](c2ccccc2)c2ccccc2)cc(C)c1OCC(=O)OC1C2CC3C(=O)OC1C3O2 ; Cc1cc([S+](c2ccccc2)c2ccccc2)cc(C)c1OCC(=O)OC1C2CC3C(=O)OC1C3C2 ; Cc1cc([S+](c2ccccc2)c2ccccc2)cc(C)c1OCC(=O)OC1C2CC3CC(C2)C(=O)OC1C3 ; Cc1cc([S+](c2ccccc2)c2ccccc2)cc(C)c1OCC(=O)O ; Cc1cc([S+](c2ccccc2)c2ccccc2)cc(C)c1O]

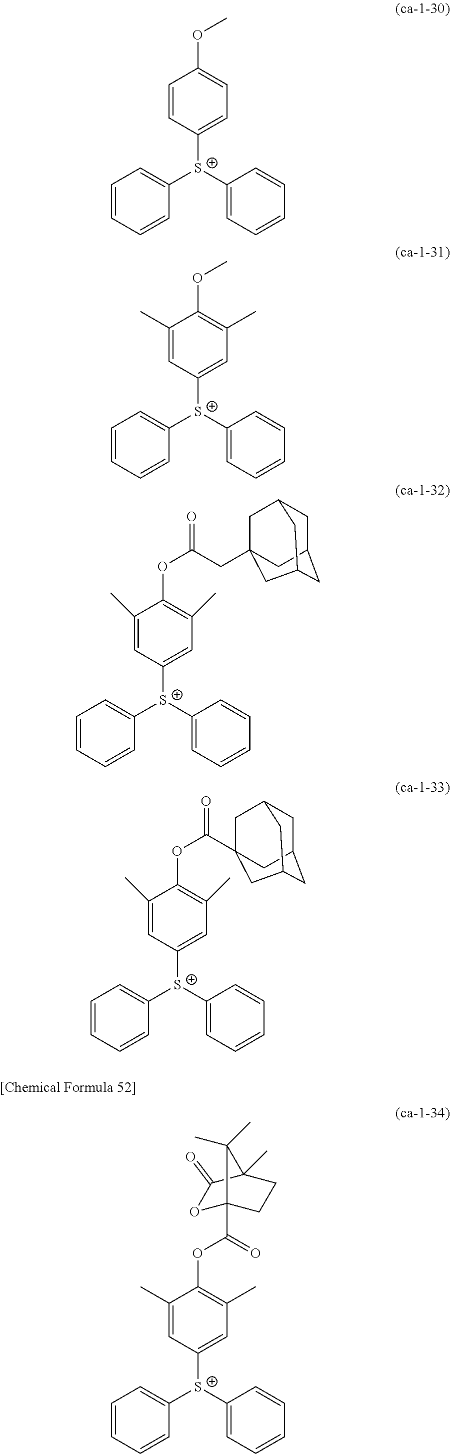 [SMILES: COc1ccc([S+](c2ccccc2)c2ccccc2)cc1 ; COc1c(C)cc([S+](c2ccccc2)c2ccccc2)cc1C ; Cc1cc([S+](c2ccccc2)c2ccccc2)cc(C)c1OC(=O)CC12CC3CC(CC(C3)C1)C2 ; Cc1cc([S+](c2ccccc2)c2ccccc2)cc(C)c1OC(=O)C12CC3CC(CC(C3)C1)C2 ; Cc1cc([S+](c2ccccc2)c2ccccc2)cc(C)c1OC(=O)C12CCC(C)(C(=O)O1)C2(C)C]

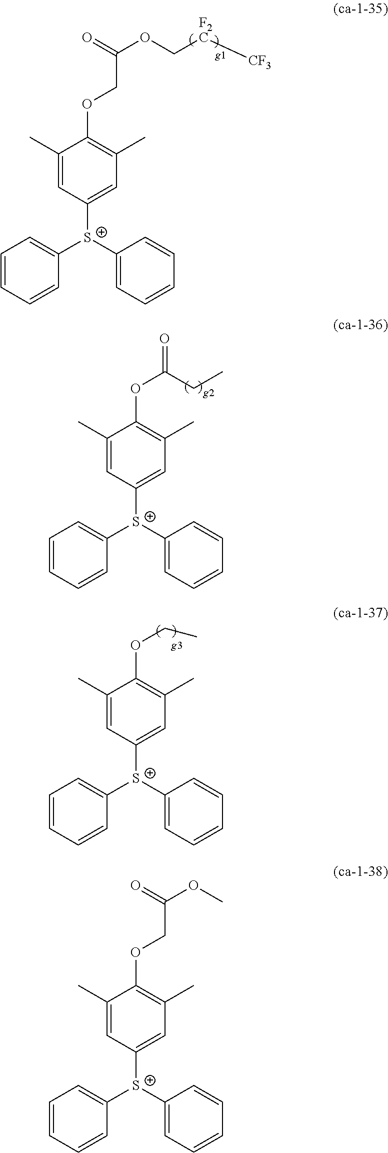 [SMILES: Cc1cc([S+](c2ccccc2)c2ccccc2)cc(C)c1OCC(=O)OCC(F)(F)C(F)(F)F ; CCC(=O)Oc1c(C)cc([S+](c2ccccc2)c2ccccc2)cc1C ; CCOc1c(C)cc([S+](c2ccccc2)c2ccccc2)cc1C ; COC(=O)COc1c(C)cc([S+](c2ccccc2)c2ccccc2)cc1C]

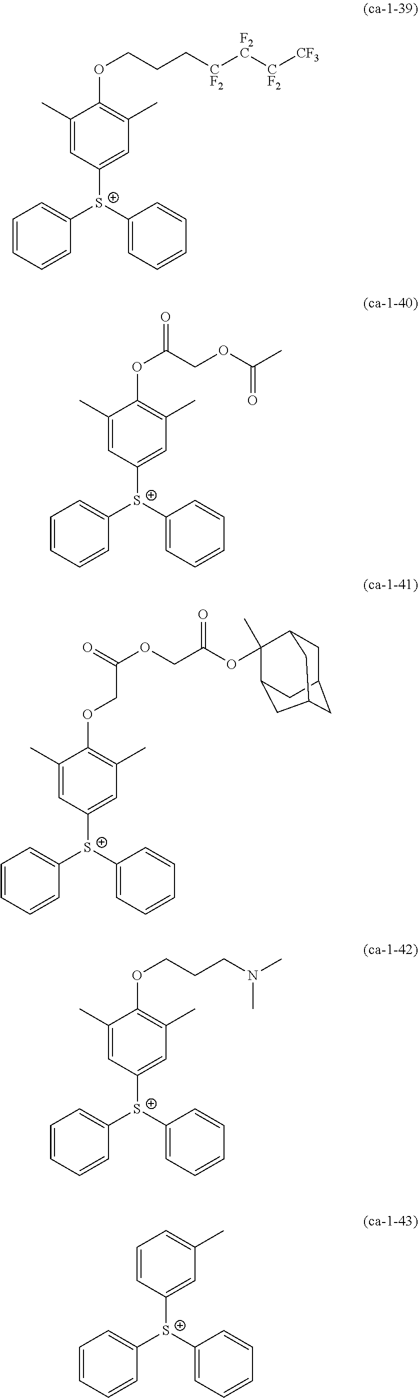 [SMILES: Cc1cc([S+](c2ccccc2)c2ccccc2)cc(C)c1OCCCC(F)(F)C(F)(F)C(F)(F)C(F)(F)F ; CC(=O)OCC(=O)Oc1c(C)cc([S+](c2ccccc2)c2ccccc2)cc1C ; Cc1cc([S+](c2ccccc2)c2ccccc2)cc(C)c1OCC(=O)OCC(=O)OC1(C)C2CC3CC(C2)CC1C3 ; Cc1cc([S+](c2ccccc2)c2ccccc2)cc(C)c1OCCCN(C)C ; Cc1cccc([S+](c2ccccc2)c2ccccc2)c1]

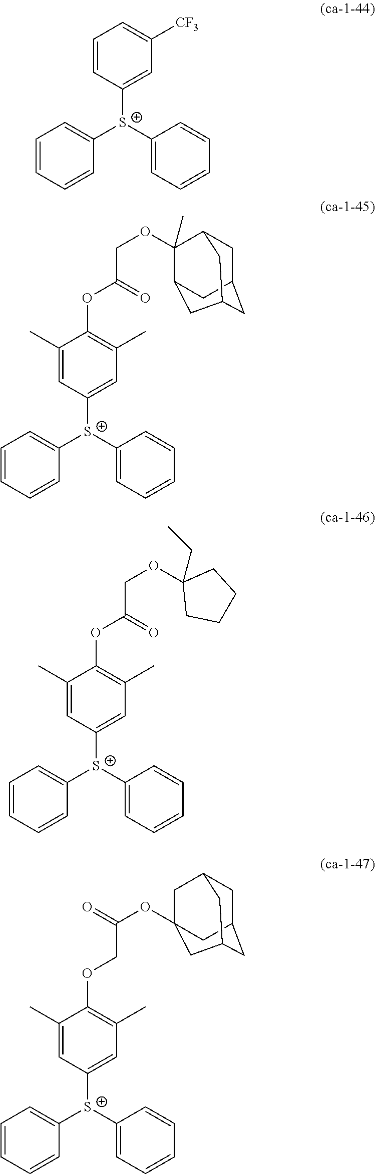 [SMILES: FC(F)(F)c1cccc([S+](c2ccccc2)c2ccccc2)c1 ; Cc1cc([S+](c2ccccc2)c2ccccc2)cc(C)c1OC(=O)COC1(C)C2CC3CC(C2)CC1C3 ; CCC1(OCC(=O)Oc2c(C)cc([S+](c3ccccc3)c3ccccc3)cc2C)CCCC1 ; Cc1cc([S+](c2ccccc2)c2ccccc2)cc(C)c1OCC(=O)OC12CC3CC(CC(C3)C1)C2]

[in the formula, g1, g2, and g3 are the number of repetitions, g1 is an integer of 1 to 5, g2 is an integer of 0 to 20, and g3 is an integer of 0 to 20].

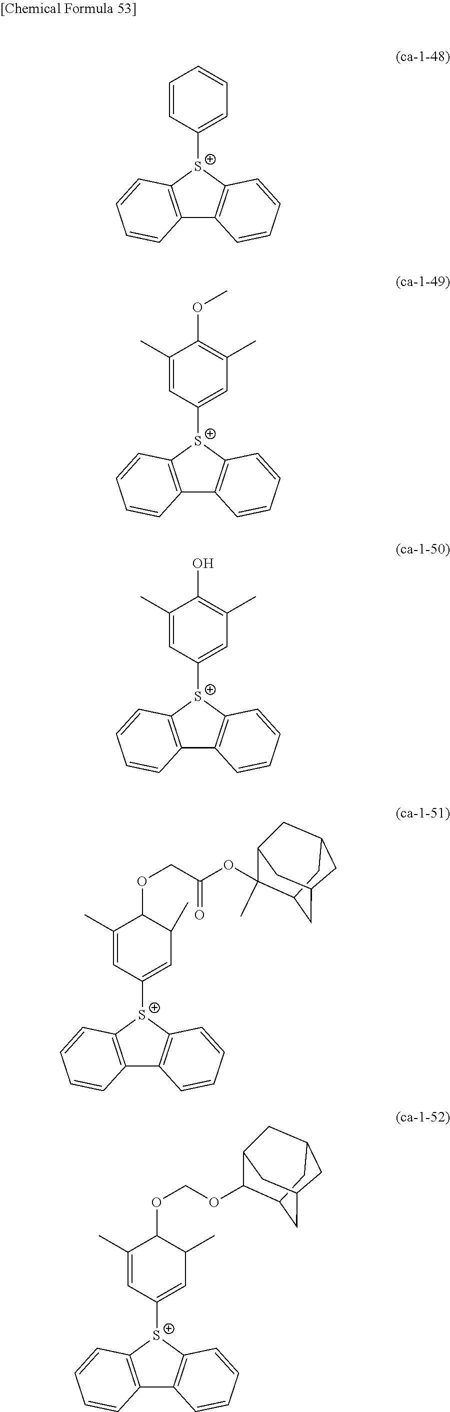 [SMILES: c1ccc(-[s+]2c3ccccc3c3ccccc32)cc1 ; COc1c(C)cc(-[s+]2c3ccccc3c3ccccc32)cc1C ; Cc1cc(-[s+]2c3ccccc3c3ccccc32)cc(C)c1O ; CC1=CC([s+]2c3ccccc3c3ccccc32)=CC(C)C1OCC(=O)OC1(C)C2CC3CC(C2)CC1C3 ; CC1=CC([s+]2c3ccccc3c3ccccc32)=CC(C)C1OCOC1C2CC3CC(C2)CC1C3]

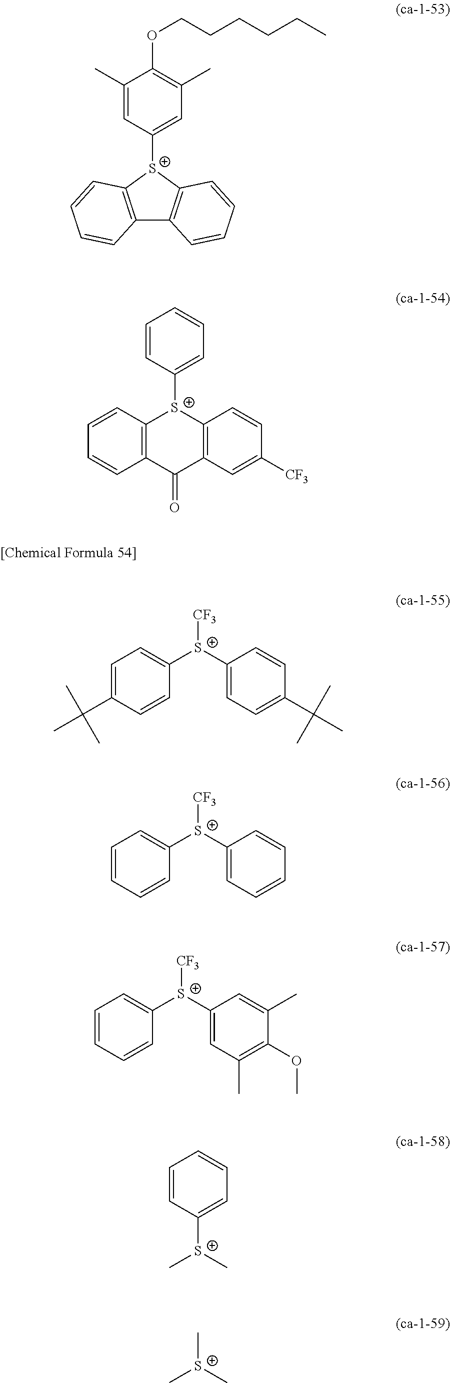

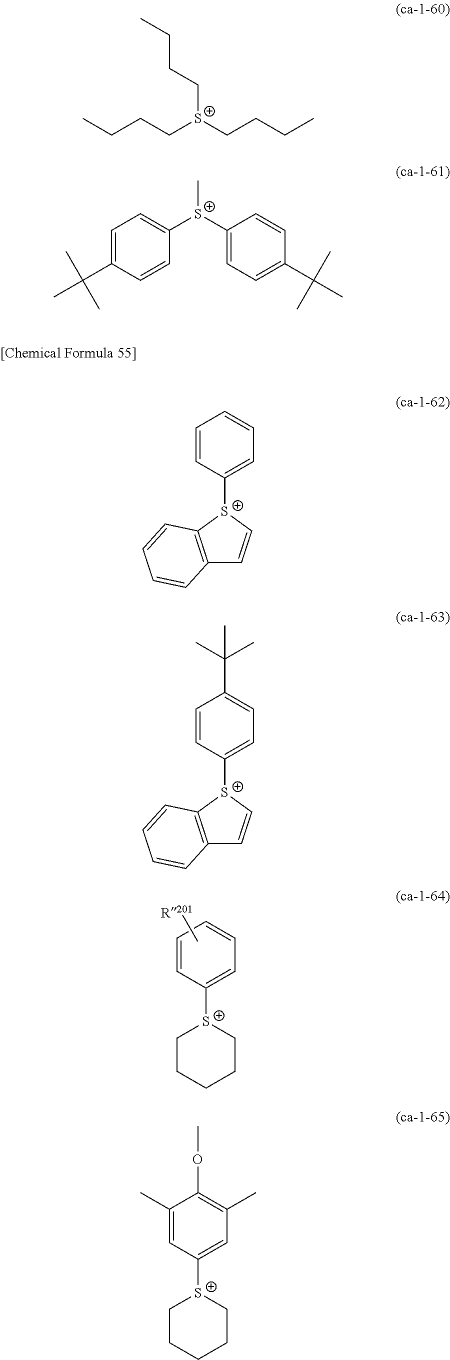

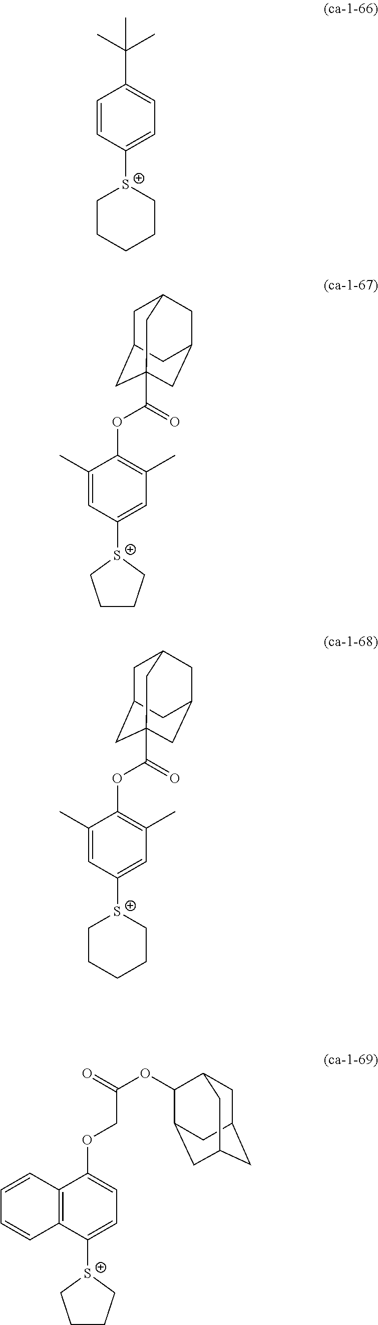 [SMILES: CC(C)(C)c1ccc([S+]2CCCCC2)cc1 ; Cc1cc([S+]2CCCC2)cc(C)c1OC(=O)C12CC3CC(CC(C3)C1)C2 ; Cc1cc([S+]2CCCCC2)cc(C)c1OC(=O)C12CC3CC(CC(C3)C1)C2 ; O=C(COc1ccc([S+]2CCCC2)c2ccccc12)OC1C2CC3CC(C2)CC1C3]

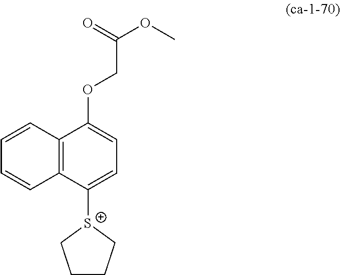 [SMILES: COC(=O)COc1ccc([S+]2CCCC2)c2ccccc12]

[in the formula, R″²⁰¹ is a hydrogen atom or a substituent, and the substituents are the same as those exemplified as the substituents that R²⁰¹ to R²⁰⁷, and R²¹⁰ to R²¹² may have].

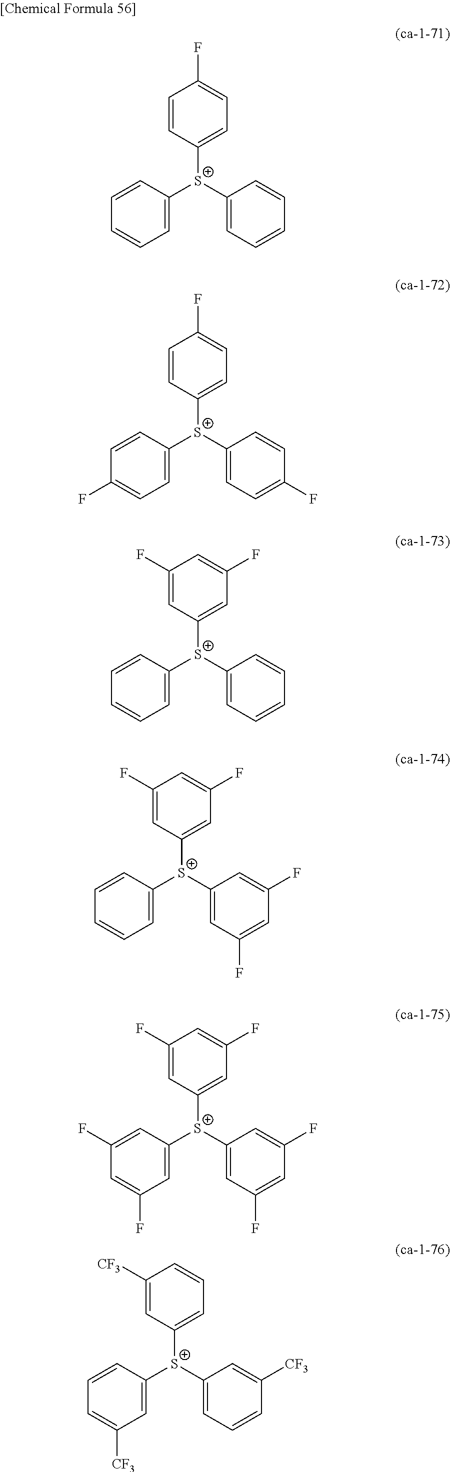 [SMILES: Fc1ccc([S+](c2ccccc2)c2ccccc2)cc1 ; Fc1ccc([S+](c2ccc(F)cc2)c2ccc(F)cc2)cc1 ; Fc1cc(F)cc([S+](c2ccccc2)c2ccccc2)c1 ; Fc1cc(F)cc([S+](c2ccccc2)c2cc(F)cc(F)c2)c1 ; Fc1cc(F)cc([S+](c2cc(F)cc(F)c2)c2cc(F)cc(F)c2)c1 ; FC(F)(F)c1cccc([S+](c2cccc(C(F)(F)F)c2)c2cccc(C(F)(F)F)c2)c1]

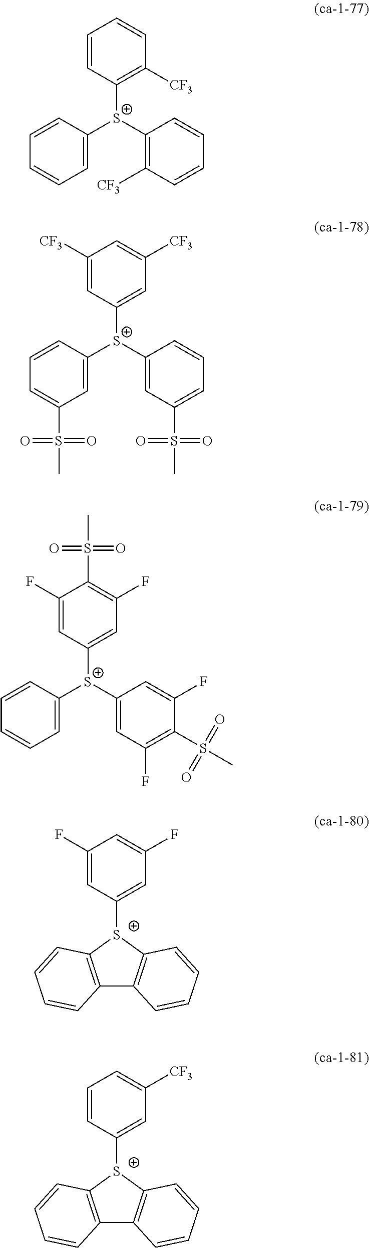 [SMILES: FC(F)(F)c1ccccc1[S+](c1ccccc1)c1ccccc1C(F)(F)F ; CS(=O)(=O)c1cccc([S+](c2cc(C(F)(F)F)cc(C(F)(F)F)c2)c2cccc(S(C)(=O)=O)c2)c1 ; CS(=O)(=O)c1c(F)cc([S+](c2ccccc2)c2cc(F)c(S(C)(=O)=O)c(F)c2)cc1F ; Fc1cc(F)cc(-[s+]2c3ccccc3c3ccccc32)c1 ; FC(F)(F)c1cccc(-[s+]2c3ccccc3c3ccccc32)c1]

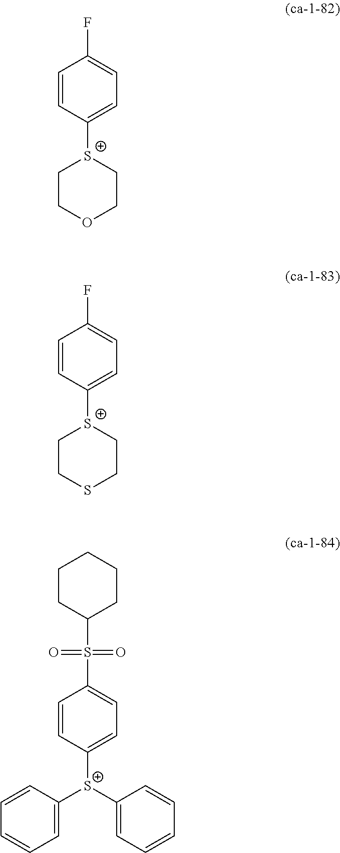 [SMILES: Fc1ccc([S+]2CCOCC2)cc1 ; Fc1ccc([S+]2CCSCC2)cc1 ; O=S(=O)(c1ccc([S+](c2ccccc2)c2ccccc2)cc1)C1CCCCC1]

Specific examples of preferable cations represented by Formula (ca-2) include diphenyliodonium cations and bis(4-tert-butylphenyl)iodonium cations.

Specific examples of preferable cations represented by Formula (ca-3) include cations represented by the following Formulae (ca-3-1) to (ca-3-6).

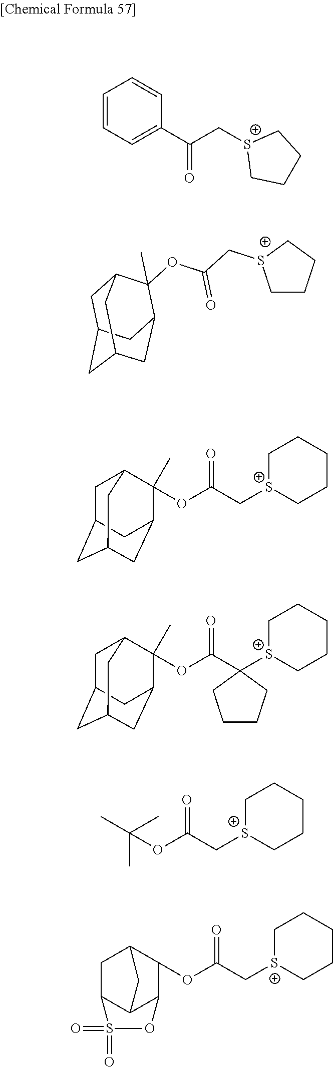 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

The cation moiety ((M′^{m+})_{1/m}) in Formula (a5-1) is preferably a sulfonium cation, more preferably a cation represented by each of Formulae (ca-1) to (ca-3), still more preferably a cation represented by Formula (ca-1), and particularly preferably a cation represented by each of Formulae (ca-1-1) to (ca-1-84).

Particularly, in order to achieve high sensitivity, as the preferable cation represented by Formula (ca-1), one having an electron-withdrawing group such as a fluorine atom, a fluorinated alkyl group, or a sulfonyl group, as a substituent, is preferable, and for example, a cation selected from the group consisting of cations represented by Chemical Formulae (ca-1-44), and (ca-1-71) to (ca-1-84) is particularly preferable.

Preferable specific examples of structural units (a5) are shown below.

In the following formulae, R^{α} is a hydrogen atom, a methyl group or a trifluoromethyl group. m and M′^{m+} are the same as m and M′^{m+} in General Formula (a5-1).

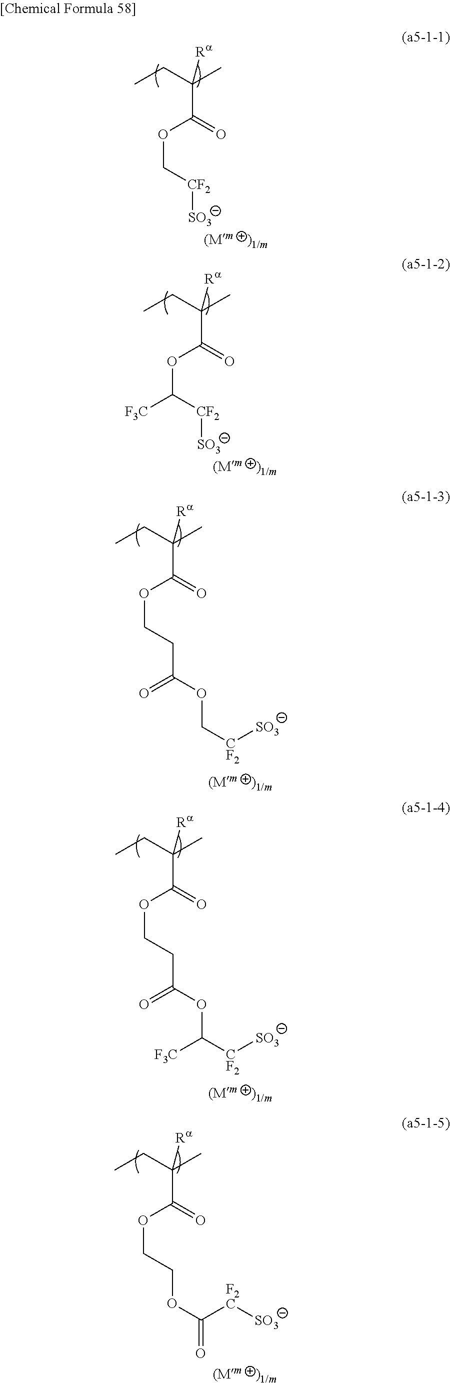

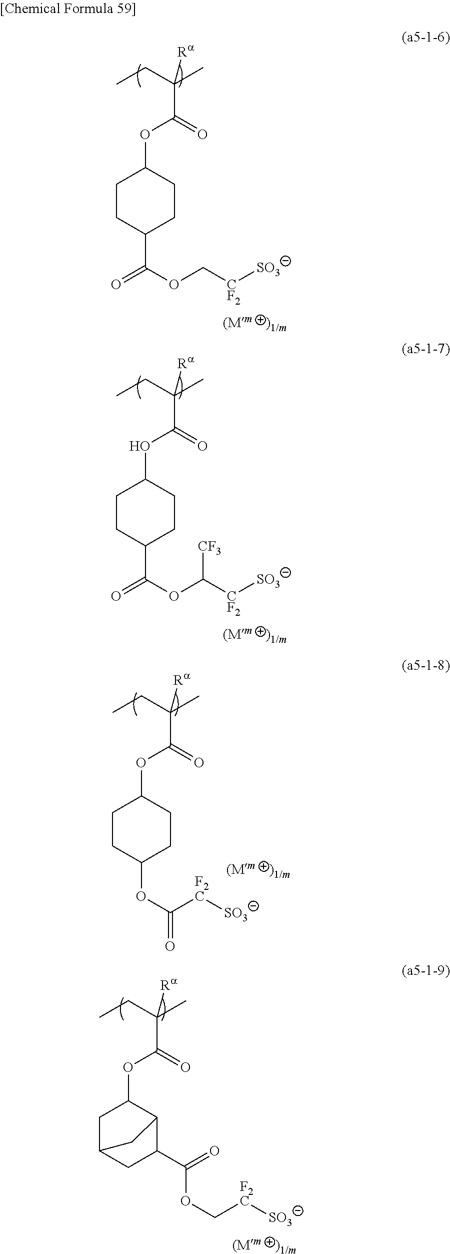

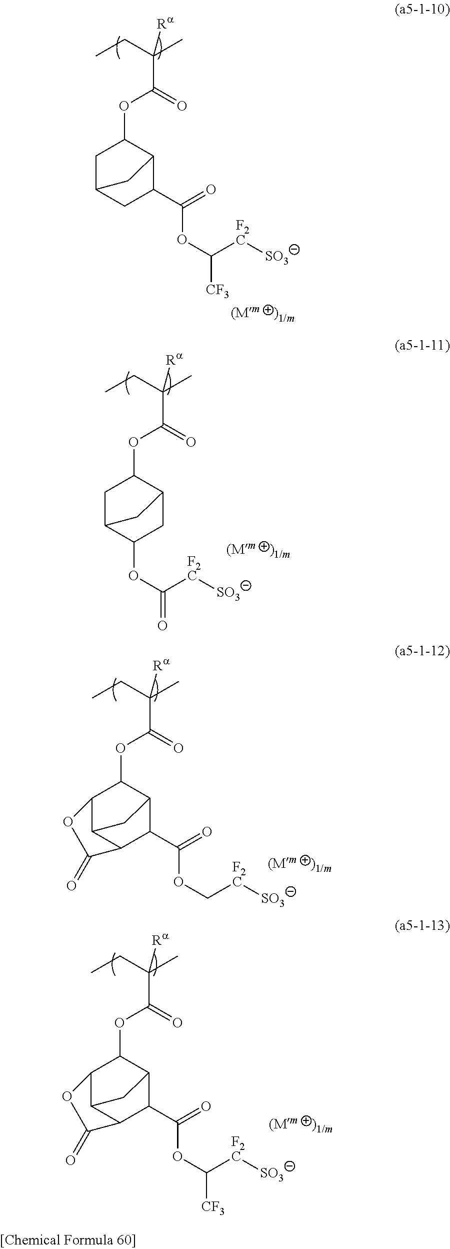

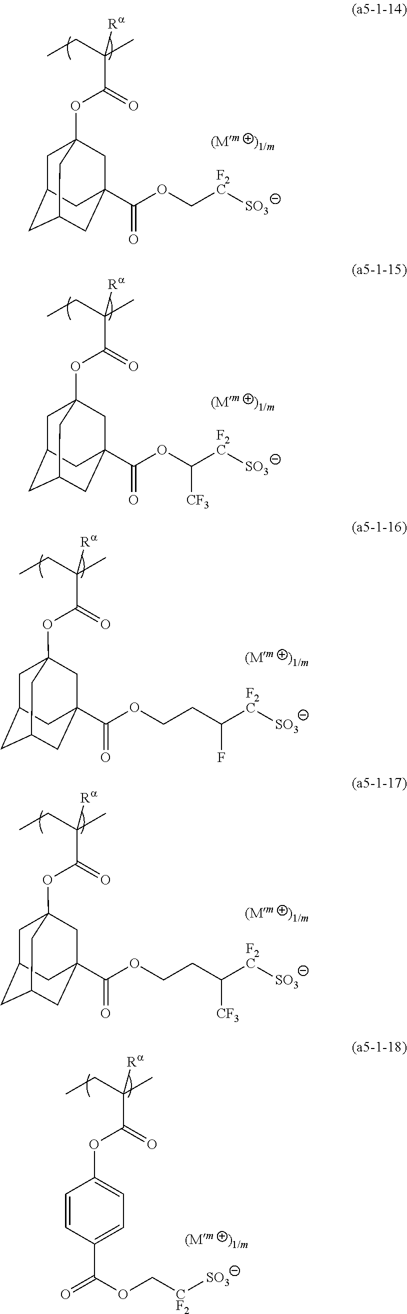

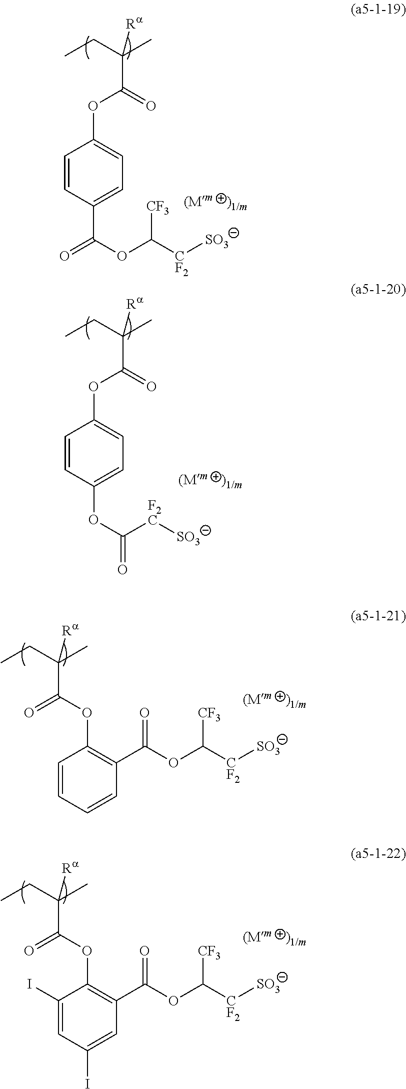

The structural unit (a5) of the component (A1) may be of one type or of two or more types.

When the component (A1) has the structural unit (a5), the proportion of the structural unit (a5) in the component (A1) based on a total amount (100 mol %) of all structural units constituting the component (A1) is preferably 5 to 25 mol %, more preferably 10 to 20 mol %, and still more preferably 15 to 20 mol %.

When the proportion of the structural unit (a5) is set to be equal to or larger than the lower limit value of the preferable range, it becomes easier to achieve higher sensitivity and improved resolution. On the other hand, when the proportion is set to be equal to or smaller than the upper limit value of the preferable range, it becomes easier to achieve a balance with other structural units.

### Structural Unit (a6):

The structural unit (a6) is a structural unit having an ability to control acid diffusion. The component (A1) may or may not have the structural unit (a6). As the structural unit (a6), known structural units can be used. Examples of structural units (a6) include structural units having structures described in the following component (D1) and component (D2). Examples thereof include structural units having structures represented by any of the following General Formulae (d1-1) to (d1-3).

The structural unit (a6) of the component (A1) may be of one type or of two or more types.

When the component (A1) has the structural unit (a6), the proportion of the structural unit (a6) in the component (A1) based on a total amount (100 mol %) of all structural units constituting the component (A1) is preferably 1 to 20 mol %, more preferably 2 to 15 mol %, and still more preferably 3 to 10 mol %.

When the proportion of the structural unit (a6) is set to be equal to or larger than the lower limit value of the preferable range, it becomes easier to achieve higher sensitivity. On the other hand, when the proportion is set to be equal to or smaller than the upper limit value of the preferable range, it becomes easier to achieve a balance with other structural units.

### Structural Unit (a8):

The structural unit (a8) is a structural unit derived from the compound represented by the following General Formula (a8-1). The component (A1) may or may not have the structural unit (a8).

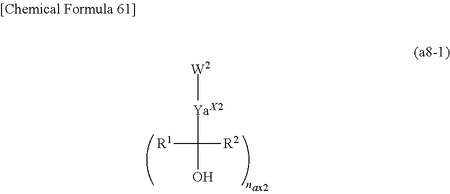

[in the formula, W² is a polymerizable group-containing group, Ya^{x2} is a single bond or a (nₐₓ₂+1)-valent linking group, Ya^{x2} and W² may form a fused ring, R¹ is a fluorinated alkyl group having 1 to 12 carbon atoms, R² is an organic group having 1 to 12 carbon atoms, which may have a fluorine atom or a hydrogen atom, R² and Ya^{x2} may be bonded to each other to form a ring structure, and nₐₓ₂ is an integer of 1 to 3].

The “polymerizable group” in the polymerizable group-containing group for W² is a group that enables a compound having a polymerizable group to be polymerized by radical polymerization or the like, and is, for example, a group containing a multiple bond between carbon atoms such as an ethylenic double bond.

The polymerizable group-containing group may be a group composed of only a polymerizable group or may be a group composed of a polymerizable group and a group other than the polymerizable group. Examples of groups other than the polymerizable group include a divalent hydrocarbon group which may have a substituent and a heteroatom-containing divalent linking group.

Preferable examples of polymerizable group-containing groups include groups represented by chemical formula: C(R^{X11})(R^{X12})═C(R^{X13})—Ya^{x0}—.

In the chemical formula, R^{X11}, R^{X12} and R^{X13} are each hydrogen atom, an alkyl group having 1 to 5 carbon atoms or a halogenated alkyl group having 1 to 5 carbon atoms, and Ya^{x0} is a single bond or a divalent linking group.

Examples of fused rings formed by Ya^{x2} and W² include a fused ring formed by a polymerizable group at the W² moiety and Ya^{x2} and a fused ring formed by a group other than the polymerizable group at the W² moiety and Ya^{x2}.

The fused ring formed by Ya^{x2} and W² may have a substituent.

Specific examples of structural units (a8) are shown below.

In the following formulae, R^{α} is a hydrogen atom, a methyl group or a trifluoromethyl group.

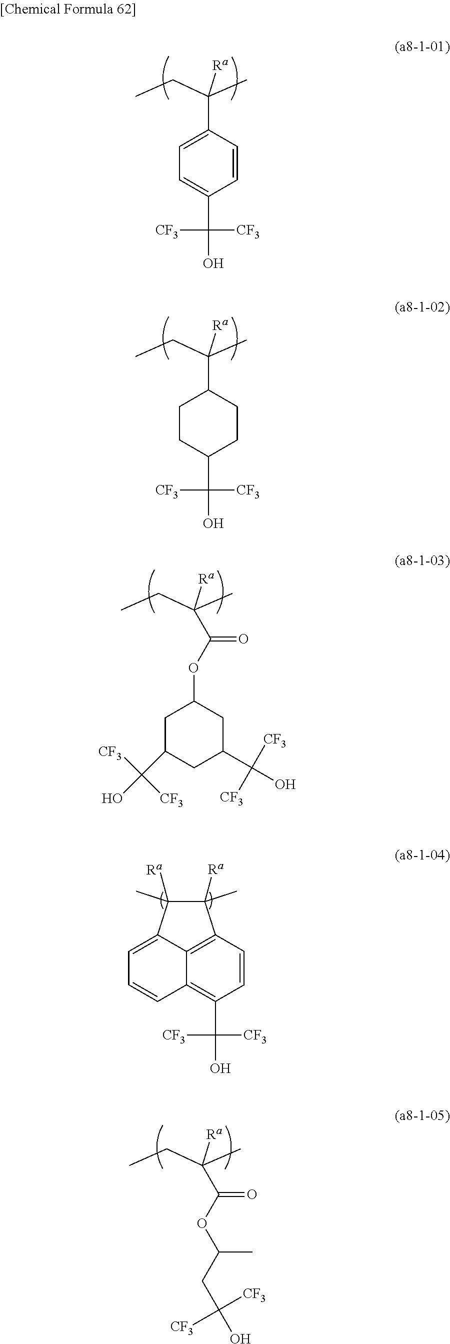

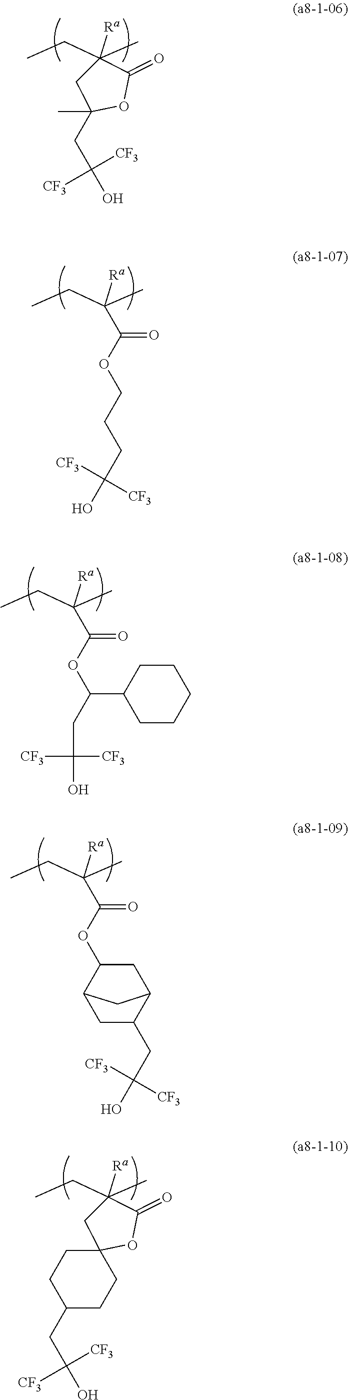

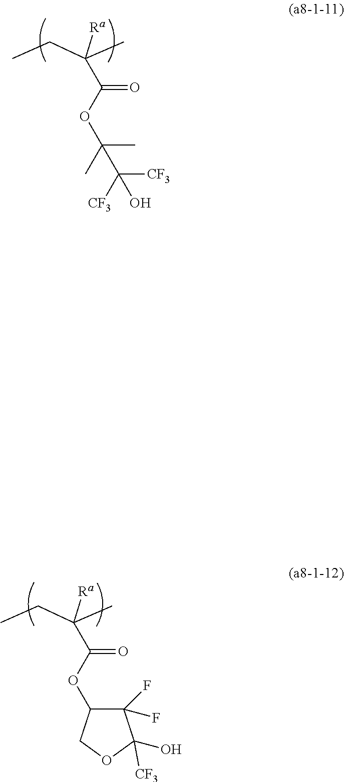

Among the above examples, the structural unit (a8) is preferably at least one selected from the group consisting of structural units represented by Chemical Formulae (a8-1-01) to (a8-1-04), (a8-1-06), (a8-1-08), (a8-1-09), and (a8-1-10), and more preferably at least one selected from the group consisting of structural units represented by Chemical Formulae (a8-1-01) to (a8-1-04), and (a8-1-09).

The structural unit (a8) of the component (A1) may be of one type or two or more types. The component (A1) may or may not have the structural unit (a8).

The proportion of the structural unit (a8) in the component (A1) based on a total amount (100 mol %) of all structural units constituting the component (A1) is preferably 0 to 50 mol % and more preferably 0 to 30 mol %.

The components (A1) in the resist composition may be used alone or two or more thereof may be used in combination.

Examples of components (A1) include a polymer compound having a repeating structure of the structural unit (a1) and the structural unit (a10).

In the polymer compound having a repeating structure of the structural unit (a1) and the structural unit (a10), the proportion of the structural unit (a1) based on a total amount (100 mol %) of all structural units constituting the polymer compound is preferably 25 to 80 mol %, more preferably 10 to 75 mol %, still more preferably 30 to 70 mol %, and yet more preferably 40 to 70 mol %.

The proportion of the structural unit (a10) in the polymer compound based on a total amount (100 mol %) of all structural units constituting the polymer compound is preferably 25 to 90 mol %, more preferably 30 to 70 mol %, still more preferably 30 to 60 mol %, and particularly preferably 20 to 50 mol %.

The component (A1) can be produced by dissolving monomers from which structural units are derived in a polymerization solvent, and adding a radical polymerization initiator such as azobisisobutyronitrile (AIBN), or dimethyl azobisisobutyrate (for example, V-60l) thereto to cause polymerization.

Alternatively, the component (A1) can be produced by dissolving a monomer from which the structural unit (a1) is derived and a monomer from which any structural unit (for example, the structural unit (a10)) is derived in a polymerization solvent, causing polymerization by adding the above radical polymerization initiator thereto, and then performing a deprotection reaction.

Here, during polymerization, for example, when a chain transfer agent such as HS—CH₂—CH₂—CH₂—C(CF₃)₂—OH is used in combination, a —C(CF₃)₂—OH group may be introduced into the end. Accordingly, a copolymer in which a hydroxyalkyl group in which some hydrogen atoms in the alkyl group are substituted with fluorine atoms is introduced is effective in reducing development defects and reducing line edge roughness (LER: non-uniform unevenness of line sidewalls).

The weight average molecular weight (Mw) (in terms of polystyrene determined through gel permeation chromatography (GPC)) of the component (A1) is not particularly limited, and is preferably 1,000 to 50,000, more preferably 5,000 to 40,000, and still more preferably 5,000 to 30,000.

When the Mw of the component (A1) is equal to or smaller than the preferable upper limit value in this range, the component has sufficient solubility in a resist solvent for use as a resist, and when the Mw of the component (A1) is equal to or larger than the preferable lower limit value in this range, the component has favorable dry etching resistance and a favorable cross-sectional shape of the resist pattern.

The dispersity (Mw/Mn) of the component (A1) is not particularly limited, and is preferably 1.0 to 4.0, more preferably 1.0 to 3.0, and particularly preferably 1.0 to 2.0. Here, Mn is the number average molecular weight.

### Component (A2)

In the resist composition of the present embodiment, as the component (A), a base material component (hereinafter referred to as a “component (A2)”) that does not correspond to the component (A1) and whose solubility in a development solution changes under the action of an acid may be used in combination.

The component (A2) is not particularly limited, and any component selected from among many components conventionally known as base material components for chemically amplified resist compositions may be used.

As the component (A2), a high-molecular-weight compound or a low-molecular-weight compound may be used alone or two or more thereof may be used in combination.

The proportion of the component (A1) in the component (A) based on a total mass of the component (A) is preferably 25 mass % or more, more preferably 50 mass % or more, still more preferably 75 mass % or more, and may be 100 mass %. When the proportion is 25 mass % or more, it is easier to form a resist pattern having various excellent lithography properties such as high sensitivity and resolution, and improved CDU.

In the resist composition of the present embodiment, the content of the component (A) may be adjusted depending on the thickness of the resist film to be formed and the like.

### <Acid Generator Component (B)>

The resist composition of the present embodiment contains an acid generator component (B) that generates an acid upon exposure (component (B)).

### <<Component (B0)>>

The component (B) includes a compound (B0) represented by the following General Formula (b0) (hereinafter referred to as a “component (B0)”).

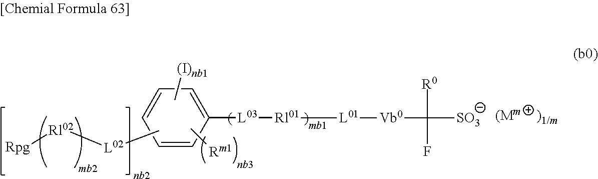

[in the formula, Rpg is an acid-decomposable group, R1⁰¹ and R1⁰² are each independently a cyclic hydrocarbon group which may have a substituent, L⁰¹ is a divalent linking group or a single bond, L⁰² and L⁰¹ are each independently a divalent linking group, R^{m1} is a substituent other than an iodine atom, Vb⁰ is a single bond, an alkylene group or a fluorinated alkylene group, R⁰ is a hydrogen atom, a fluorinated alkyl group having 1 to 5 carbon atoms or a fluorine atom, nb1 is an integer of 1 to 4, nb2 is an integer of 1 to 4, nb3 is an integer of 0 to 3, and nb1+nb2+nb3≤5, mb1 and mb2 are each independently 0 or 1, and mb1 and mb2 are not both 0, M^{m+} is an m-valent organic cation, and m is an integer of 1 or more].

### [Anion Moiety]

In General Formula (b0), the acid-decomposable group for Rpg is a group that decomposes under the action of an acid to generate a polar group. Examples of polar groups include a carboxy group, a hydroxy group, an amino group, and a sulfo group (—SO₃H).

Rpg is preferably an acid-decomposable group represented by any of the following General Formulae (r-pg-1) to (r-pg-4).

<<Acid-Decomposable Group Represented by General Formula (r-Pg-1)>>

The acid-decomposable group represented by General Formula (r-pg-1) is a group in which a hydroxy group is protected with an “acetal-type acid-dissociable group” as described in the above structural unit (al).

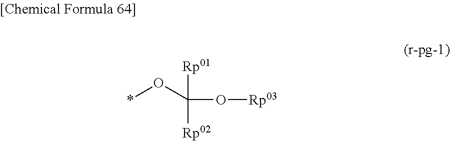

[in the formula, Rp⁰¹ and Rp⁰² are each independently a hydrogen atom or an alkyl group, Rp⁰³ is a hydrocarbon group, and Rp⁰³ may be bonded to either Rp⁰¹ or Rp⁰² to form a ring, and * indicates a bond].
<<Acid-Decomposable Group Represented by General Formula (r-Pg-2)>>

The acid-decomposable group represented by General Formula (r-pg-2) is a group in which a carboxy group is protected with an “acetal-type acid-dissociable group” as described in the above structural unit (al).

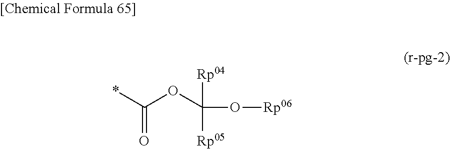

[in the formula, Rp⁰⁴ and Rp⁰⁵ are each independently a hydrogen atom or an alkyl group, Rp⁰⁶ is a hydrocarbon group, Rp⁰⁶ may be bonded to either Rp⁰⁴ or Rp⁰⁵ to form a ring, and * indicates a bond].
<<Acid-Decomposable Group Represented by General Formula (r-Pg-3)>>

The acid-decomposable group represented by General Formula (r-pg-3) is a group in which a carboxy group is protected with a “tertiary alkyl ester type acid-dissociable group” as described in the above structural unit (al).

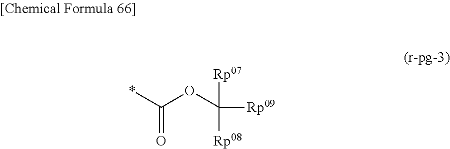

[in the formula, Rp⁰⁷ to Rp⁰⁹ are each independently a hydrocarbon group, Rp⁰⁸ and Rp⁰⁹ may be bonded to each other to form a ring, and * indicates a bond].
<<Acid-Decomposable Group Represented by General Formula (r-Pg-4)>>

The acid-decomposable group represented by General Formula (r-pg-4) is a group in which a carboxy group is protected with a “secondary alkyl ester type acid-dissociable group” as described in the above structural unit (al).

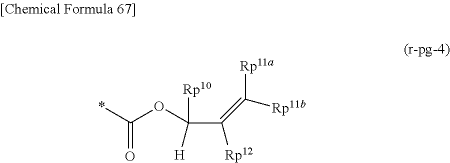

[in the formula, Rp¹⁰ is a hydrocarbon group, Rp^{11a} and Rp^{11b} are each independently a hydrogen atom, halogen atom or an alkyl group, Rp¹² is a hydrogen atom or a hydrocarbon group, Rp¹⁰ and Rp^{11a} or Rp^{11b} may be bonded to each other to form a ring, Rp^{11a} or Rp^{11b} and Rp¹² may be bonded to each other to form a ring, and * indicates a bond].

In Formula (b0), Rpg is preferably an acid-decomposable group represented by General Formula (r-pg-3), and more preferably an acid-decomposable group represented by any of the following General Formulae (r-pg-3-01) to (r-pg-3-04).

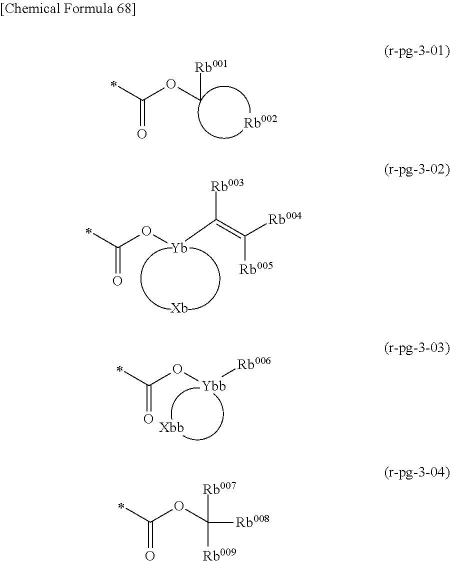

[in Formula (r-pg-3-01), Rb⁰⁰¹ is a linear or branched alkyl group having 1 to 12 carbon atoms, some of which may be substituted with a halogen atom or a heteroatom-containing group, Rb⁰⁰² is a group that forms an aliphatic cyclic group together with a carbon atom to which Rb⁰⁰¹ is bonded,

in Formula (r-pg-3-02), Yb is a carbon atom, Xb is a group which forms a cyclic organic group together with Yb, some or all of the hydrogen atoms of the cyclic organic group may be substituted, Rb⁰⁰³ to Rb⁰⁰⁵ are each independently a hydrogen atom, a monovalent chain saturated hydrocarbon group having 1 to 10 carbon atoms or a monovalent aliphatic cyclic saturated hydrocarbon group having 3 to 20 carbon atoms, some or all of the hydrogen atoms in the chain saturated hydrocarbon group and the aliphatic cyclic saturated hydrocarbon group may be substituted, two or more of Rb⁰⁰³ to Rb⁰⁰⁵ may be bonded to each other to form a ring structure,

in Formula (r-pg-3-03), Ybb is a carbon atom, Xbb is a group which forms an aliphatic cyclic group together with Ybb, Rb⁰⁰⁶ is an aromatic hydrocarbon group which may have a substituent,

in Formula (r-pg-3-04), Rb⁰⁰⁷ and Rb⁰⁰⁸ are each independently a monovalent chain hydrocarbon group having 1 to 10 carbon atoms, some or all of the hydrogen atoms of the chain hydrocarbon group may be substituted, Rb⁰⁰⁹ is a hydrocarbon group which may have a substituent, and * indicates a bond (the same applies hereinafter)].

Acid-Decomposable Group Represented by General Formula (r-Pg-3-01)

In Formula (r-pg-3-01), the linear alkyl group for Rb⁰⁰′ preferably has 1 to 10 carbon atoms and particularly preferably has 1 to 5 carbon atoms.

The branched alkyl group for Rb⁰⁰¹ has, for example, 3 to 12 carbon atoms, preferably has 3 to 10 carbon atoms, and more preferably has 3 to 5 carbon atoms. Examples of branched alkyl groups include an isopropyl group, an isobutyl group, a tert-butyl group, an isopentyl group, a neopentyl group, a 1,1-diethylpropyl group, and a 2,2-dimethylbutyl group.

Some carbon atoms in the alkyl group for Rb⁰⁰¹ may be substituted with a halogen atom or a heteroatom-containing group. For example, some hydrogen atoms constituting the alkyl group may be substituted with a halogen atom or a heteroatom-containing group. In addition, some carbon atoms (a methylene group, etc.) constituting the alkyl group may be substituted with a heteroatom-containing group.

Examples of heteroatoms referred to here include an oxygen atom, a sulfur atom, and a nitrogen atom. Examples of heteroatom-containing groups include —O—, —C(═O)—O—, —O—C(═O)—, —C(═O)—, —O—C(═O)—O—, —C(═O)—NH—, —NH—, —S—, —S(═O)₂—, and —S(═O)₂—O—.

Among the above examples, Rb⁰⁰¹ in Formula (r-pg-3-01) is preferably a linear or branched alkyl group having 1 to 5 carbon atoms, more preferably a linear alkyl group having 1 to 5 carbon atoms, and still more preferably a methyl group or an ethyl group.

In Formula (r-pg-3-01), Rb⁰⁰² (an aliphatic cyclic group formed together with a carbon atom to which Rb⁰⁰¹ is bonded) is, for example, an alicyclic hydrocarbon group which is a monocyclic group or a polycyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane, and the polycycloalkane is preferably one having 7 to 12 carbon atoms, and specific examples thereof include adamantane, norbornane, isobornane, tricyclodecane, and tetracyclododecane.

Among the above examples, Rb⁰⁰² is preferably a cyclopentyl group, a cyclohexyl group, an adamantyl group, or a norbornyl group, and more preferably a cyclopentyl group or an adamantyl group.

Preferable specific examples of acid-decomposable groups represented by General Formula (r-pg-3-01) are shown below.

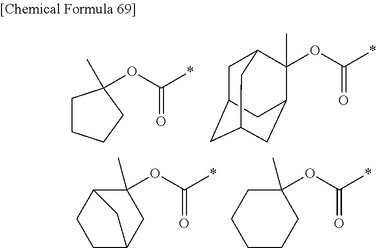

Acid-Decomposable Group Represented by General Formula (r-Pg-3-02)

In Formula (r-pg-3-02), a cyclic organic group formed by Xb together with Yb is, for example, an alicyclic hydrocarbon group which is a monocyclic group or a polycyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane. The polycycloalkane is preferably one having 7 to 12 carbon atoms, and specific examples thereof include adamantane, norbornane, isobornane, tricyclodecane, and tetracyclododecane.

The cyclic organic group formed by Xb together with Yb may contain a heteroatom such as in a heterocycle. Examples of heteroatoms include an oxygen atom, a sulfur atom, and a nitrogen atom. Specific examples of heterocycles include aliphatic heterocycles such as tetrahydrofuran, tetrahydropyran, and tetrahydrothiophene.

The cyclic organic group formed by Xb together with Yb may have a substituent. Examples of such substituents include those described above for Ra^{x5}.

In Formula (r-pg-3-02), the cyclic organic group formed by Xb together with Yb is preferably an alicyclic hydrocarbon group which is a monocyclic group or an aliphatic heterocyclic hydrocarbon group which is a monocyclic group, and more preferably a cyclopentyl group, a cyclohexyl group, or a tetrahydrofuranyl group.

In Formula (r-pg-3-02), examples of monovalent chain saturated hydrocarbon groups having 1 to 10 carbon atoms for Rb⁰⁰³ to Rb⁰⁰⁵ include a methyl group, an ethyl group, a propyl group, a butyl group, a pentyl group, a hexyl group, a heptyl group, an octyl group, and a decyl group.

Examples of monovalent aliphatic cyclic saturated hydrocarbon groups having 3 to 20 carbon atoms for Rb⁰⁰³ to Rb⁰⁰⁵ include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.0^{2,6}]decanyl group, a tricyclo[3.3.1.1^{3,7}]decanyl group, a tetracyclo[6.2.1.1^{3,6}.0^{2,7}]dodecanyl group, and an adamantyl group.

Examples of substituents that the chain saturated hydrocarbon groups or aliphatic cyclic saturated hydrocarbon groups represented by Rb⁰⁰³ to Rb⁰⁰⁵ include the same groups as for Ra^{x5}.

Examples of groups containing a carbon-carbon double bond generated when two or more of Rb⁰⁰³ to Rb⁰⁰⁵ are bonded to each other to form a ring structure include a cyclopentenyl group, a cyclohexenyl group, a methylcyclopentenyl group, a methylcyclohexenyl group, a cyclopentylideneethenyl group, and a cyclohexylideneethenyl group. Among these, in consideration of ease of synthesis, a cyclopentenyl group, a cyclohexenyl group, or a cyclopentylideneethenyl group is preferable.

Among the above examples, in consideration of ease of synthesis, Rb⁰⁰³ to Rb⁰⁰⁵ are preferably a hydrogen atom or a monovalent chain saturated hydrocarbon group having 1 to 10 carbon atoms, and more preferably a hydrogen atom, a methyl group, or an ethyl group.

More specifically, it is preferable that all of Rb⁰⁰³ to Rb⁰⁰⁵ be a hydrogen atom, Rb⁰⁰³ and Rb⁰⁰⁴ be a hydrogen atom, and Rb⁰⁰⁵ be a methyl group or an ethyl group.

Preferable specific examples of acid-decomposable groups represented by General Formula (r-pg-3-02) are shown below.

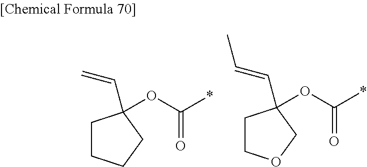

Acid-Decomposable Group Represented by General Formula (r-Pg-3-03)

In Formula (r-pg-3-03), the aliphatic cyclic group formed by Xbb together with Ybb may be a monocyclic group or a polycyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane, and the polycycloalkane is preferably one having 7 to 12 carbon atoms, and specific examples thereof include adamantane, norbornane, isobornane, tricyclodecane, and tetracyclododecane.

The aliphatic cyclic group formed by Xbb together with Ybb may contain a heteroatom such as in a heterocycle. Examples of heteroatoms include an oxygen atom, a sulfur atom, and a nitrogen atom. Specific examples of heterocycles include aliphatic heterocycles such as tetrahydrofuran, tetrahydropyran, and tetrahydrothiophene.

The cyclic hydrocarbon group formed by Xbb together with Ybb may have a substituent. Examples of such substituents include those described above for Ra^{x5}.

The aliphatic cyclic group formed by Xbb together with Ybb is preferably an alicyclic hydrocarbon group which is a monocyclic group or an aliphatic heterocyclic hydrocarbon group which is a monocyclic group, and more preferably a cyclopentyl group, a cyclohexyl group, or a tetrahydrofuranyl group.

In Formula (r-pg-3-03), examples of aromatic hydrocarbon groups for Rb⁰⁰⁶ include a group in which one or more hydrogen atoms have been removed from an aromatic hydrocarbon ring having 5 to 30 carbon atoms. Among these, Rb⁰⁰⁶ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from benzene or naphthalene, and most preferably a group in which one or more hydrogen atoms have been removed from benzene.

The aromatic hydrocarbon ring may contain a heteroatom such as in a heterocycle. Examples of heteroatoms include an oxygen atom, a sulfur atom, and a nitrogen atom. Specific examples of heterocycles include a pyridine ring and a thiophene ring.

Examples of substituents that Rb⁰⁰⁶ may have include a methyl group, an ethyl group, a propyl group, a hydroxy group, a carboxy group, a halogen atom, an alkoxy group (a methoxy group, an ethoxy group, a propoxy group, a butoxy group, etc.), and an alkyloxycarbonyl group.

Among the above examples, the aromatic hydrocarbon group for Rb⁰⁰⁶ is preferably a phenyl group or a thiophenyl group.

Preferable specific examples of acid-decomposable groups represented by General Formula (r-pg-3-03) are shown below.

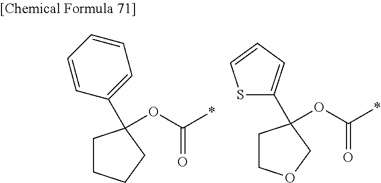

Acid-Decomposable Group Represented by General Formula (r-Pg-3-04)

In Formula (r-pg-3-04), Rb⁰⁰⁷ and Rb⁰⁰⁸ are each independently a monovalent chain hydrocarbon group having 1 to 10 carbon atoms. Some or all of the hydrogen atoms in the monovalent chain hydrocarbon group may be substituted with a substituent. Examples of such substituents include those described above for Ra^{x5}.

Examples of monovalent chain hydrocarbon groups include a linear or branched saturated hydrocarbon group (alkyl group) and a linear or branched unsaturated hydrocarbon group.

The linear alkyl group preferably has 1 to 6 carbon atoms, more preferably has 1 to 4 carbon atoms, and still more preferably 1 to 3 carbon atoms. The branched alkyl group preferably has 3 to 6 carbon atoms and more preferably has 3 or 4 carbon atoms. Specific examples of linear or branched alkyl groups include a methyl group, an ethyl group, a propyl group, an isopropyl group, an n-butyl group, an isobutyl group, a tert-butyl group, a pentyl group, an isopentyl group, and a neopentyl group.

More specific examples of linear or branched unsaturated hydrocarbon groups include unsaturated hydrocarbon groups having a double bond such as an alkenyl group, an alkadienyl group, and an alkatrienyl group; and unsaturated hydrocarbon groups having a triple bond such as an alkynyl group, a group in which one hydrogen atom has been removed from an alkyne, and a group in which one hydrogen atom has been removed from a trialkyne.

Specific examples of linear or branched alkenyl groups include linear alkenyl groups such as a vinyl group, a propenyl group (allyl group), and a 2-butenyl group; and branched alkenyl groups such as a 1-methylvinyl group, a 2-methylvinyl group, a 1-methylpropenyl group, and a 2-methylpropenyl group.

Specific examples of alkadienyl groups include a propadienyl group and a butadienyl group.

Specific examples of alkatrienyl groups include a butatrienyl group.

Specific examples of linear or branched alkynyl groups include linear alkynyl groups such as an ethynyl group, a propargyl group, a 3-pentynyl group, and a methylethynyl group (—C≡*C—CH₃); and branched alkynyl groups such as a 1-methylpropargyl group.

Specific examples of groups in which one hydrogen atom has been removed from an alkyne include a group in which one hydrogen atom has been removed from diacetylene.

Specific examples of groups in which one hydrogen atom has been removed from a trialkyne include a group in which one hydrogen atom has been removed from hexa-1,3,5-triyne.

In Formula (r-pg-3-04), Rb⁰⁰⁷ and Rb⁰⁰⁸ are each independently preferably a linear saturated hydrocarbon group having 1 to 10 carbon atoms or a linear unsaturated hydrocarbon group having 1 to 10 carbon atoms, and more preferably a linear saturated hydrocarbon group having 1 to 5 carbon atoms or a linear unsaturated hydrocarbon group having 1 to 5 carbon atoms.

In Formula (r-pg-3-04), examples of hydrocarbon groups for Rb⁰⁰⁹ include a linear or branched saturated hydrocarbon group (alkyl group), a linear or branched unsaturated hydrocarbon group, and a cyclic hydrocarbon group.

When Rb⁰⁰⁹ is a cyclic hydrocarbon group, the hydrocarbon group may be an alicyclic hydrocarbon group or an aromatic hydrocarbon group, and may be a polycyclic group or a monocyclic group.

The alicyclic hydrocarbon group which is a monocyclic group is preferably a group in which one hydrogen atom has been removed from a monocycloalkane. The monocycloalkane preferably has 3 to 6 carbon atoms, and specific examples thereof include cyclopentane and cyclohexane.

The alicyclic hydrocarbon group which is a polycyclic group is preferably a group in which one hydrogen atom has been removed from a polycycloalkane, and the polycycloalkane is preferably one having 7 to 12 carbon atoms, and specific examples thereof include adamantane, norbornane, isobornane, tricyclodecane, and tetracyclododecane.

Examples of aromatic hydrocarbon groups for Rb⁰⁰⁹ include the same aromatic hydrocarbon groups as for Rb⁰⁰⁶. Among these, Rb⁰⁰⁹ is preferably a group in which one or more hydrogen atoms have been removed from an aromatic hydrocarbon ring having 6 to 15 carbon atoms, more preferably a group in which one or more hydrogen atoms have been removed from benzene, naphthalene, anthracene or phenanthrene, still more preferably a group in which one or more hydrogen atoms have been removed from benzene, naphthalene or anthracene, particularly preferably a group in which one or more hydrogen atoms have been removed from naphthalene or anthracene, and most preferably a group in which one or more hydrogen atoms have been removed from naphthalene.

Examples of substituents that Rb⁰⁰⁹ may have include the same substituents that Rb⁰⁰⁶ may have.

Among the above examples, Rb⁰⁰⁹ is preferably a linear saturated hydrocarbon group or linear unsaturated hydrocarbon group having 1 to 10 carbon atoms or more preferably a linear saturated hydrocarbon group or linear unsaturated hydrocarbon group having 1 to 5 carbon atoms.

Preferable specific examples of acid-decomposable groups represented by General Formula (r-pg-3-04) are shown below.

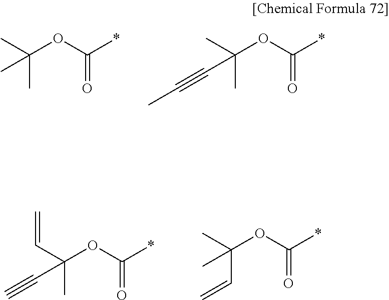

Among the above examples, Rpg is preferably an acid-decomposable group represented by General Formula (r-pg-3-01) or (r-pg-3-04), and more preferably an acid-decomposable group represented by General Formula (r-pg-3-01).

In General Formula (b0), the cyclic hydrocarbon group for R1⁰¹ and R1⁰² may be an aliphatic hydrocarbon group or an aromatic hydrocarbon group and may be a polycyclic group or a monocyclic group.

The monocyclic alicyclic group is preferably a group in which two or more hydrogen atoms have been removed from a monocycloalkane or monocycloalkene. The monocycloalkane preferably has 3 to 6 carbon atoms, and specific examples thereof include cyclopentane and cyclohexane. The monocycloalkene is preferably one having 3 to 6 carbon atoms, and specific examples thereof include cyclopentene and cyclohexene.

The polycyclic alicyclic group is preferably a group in which two or more hydrogen atoms have been removed from a polycycloalkane or polycycloalkene. The polycycloalkane is preferably one having 7 to 12 carbon atoms, and specific examples thereof include adamantane, norbornane, isobornane, tricyclodecane, and tetracyclododecane. The polycycloalkene is preferably one having 7 to 12 carbon atoms, and specific examples thereof include adamantene, norbornene, isobornene, tricyclodecene, and tetracyclododecene.

The aromatic hydrocarbon group is preferably a group in which two or more hydrogen atoms have been removed from benzene, naphthalene, anthracene, or phenanthrene, more preferably a group in which two or more hydrogen atoms have been removed from benzene, naphthalene, or anthracene, and still more preferably a group in which two or more hydrogen atoms have been removed from benzene.

Examples of substituents that the cyclic hydrocarbon group for R1⁰¹ and R1⁰² may have include the same groups as for Ra^{x5}.

In the cyclic hydrocarbon group for R1⁰¹ and R1⁰², some carbon atoms (a methylene group, etc.) constituting the hydrocarbon group may be substituted with a heteroatom-containing group.

Examples of heteroatoms referred to here include an oxygen atom, a sulfur atom, and a nitrogen atom. Examples of heteroatom-containing groups include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

That is, the cyclic hydrocarbon group which may have a substituent for R1⁰¹ and R1⁰² may be a group in which two or more hydrogen atoms have been removed from an aromatic heterocycle such as a pyridine ring or a thiophene ring; or a group in which two or more hydrogen atoms have been removed from an aliphatic heterocycle such as tetrahydrofuran, tetrahydropyran, tetrahydrothiophene, dioxolane, or dioxane.

The cyclic hydrocarbon group which may have a substituent for R1⁰¹ and R1⁰² may be a lactone-containing cyclic group such as a group represented by each of the following General Formulae (L-r-1) to (L-r-7).

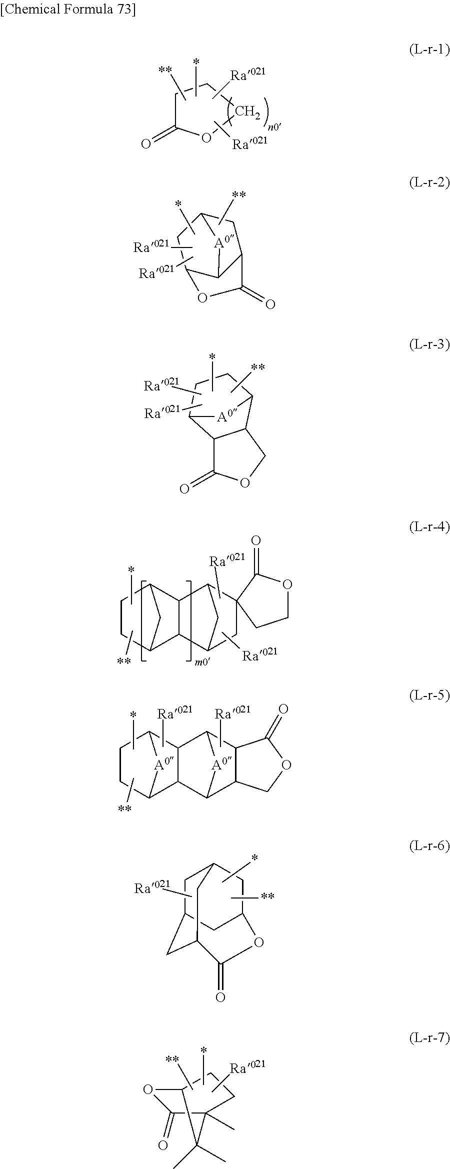

[in the formula, Ra′⁰²¹'s are each independently a hydrogen atom, an alkyl group, an alkoxy group, a halogen atom, a halogenated alkyl group, a hydroxy group, —COOR⁰″, —OC(═O)R⁰″, a hydroxyalkyl group or a cyano group; R⁰″ is a hydrogen atom, an alkyl group or a lactone-containing cyclic group; A⁰″ is an alkylene group having 1 to 5 carbon atoms which may contain an oxygen atom (—O—) or a sulfur atom (—S—), an oxygen atom, or a sulfur atom, n0′ is an integer of 0 to 2, and m0′ is 0 or 1].

In General Formulae (L-r-1) to (L-r-7), the alkyl group for Ra′⁰²¹ is preferably an alkyl group having 1 to 6 carbon atoms. The alkyl group is preferably linear or branched.

The alkoxy group for Ra′⁰²¹ is preferably an alkoxy group having 1 to 6 carbon atoms. The alkoxy group is preferably linear or branched. Specific examples thereof include a group in which an alkyl group exemplified as the alkyl group for Ra′⁰²¹ is linked to an oxygen atom (—O—).

The halogen atom for Ra′⁰²¹ is preferably a fluorine atom.

Examples of halogenated alkyl groups for Ra′⁰²¹ include a group in which some or all of the hydrogen atoms in the alkyl group for Ra′⁰²¹ are substituted with halogen atoms. The halogenated alkyl group is preferably a fluorinated alkyl group and particularly preferably a perfluoroalkyl group.

In —COOR⁰″ and —OC(═O)R⁰″ for Ra′⁰²¹, R⁰″'s are all a hydrogen atom, an alkyl group, or a lactone-containing cyclic group.

The alkyl group for R⁰″ may be linear, branched, or cyclic, and preferably has 1 to 15 carbon atoms.

When R⁰″ is a linear or branched alkyl group, it preferably has 1 to 10 carbon atoms, more preferably has 1 to 5 carbon atoms, and is particularly preferably a methyl group or an ethyl group.

When R⁰″ is a cyclic alkyl group, it preferably has 3 to 15 carbon atoms, more preferably has 4 to 12 carbon atoms, and most preferably has 5 to 10 carbon atoms.

Examples of lactone-containing cyclic groups for R⁰″ include the same groups represented by General Formulae (a2-r-1) to (a2-r-7).

The hydroxyalkyl group for Ra′⁰²¹ preferably has 1 to 6 carbon atoms, and specific examples thereof include a group in which at least one hydrogen atom in the alkyl group for Ra′⁰²¹ is substituted with a hydroxy group.

In General Formulae (L-r-2), (L-r-3), and (L-r-5), the alkylene group having 1 to 5 carbon atoms for A⁰″ is preferably a linear or branched alkylene group, and examples thereof include a methylene group, an ethylene group, an n-propylene group, and an isopropylene group. When the alkylene group contains an oxygen atom or a sulfur atom, specific examples thereof include a group in which —O— or —S— is inserted into the end or between carbon atoms of the alkylene group, and for example, —O—CH₂—, —CH₂—O—CH₂—, —S—CH₂—, and —CH₂—S—CH₂— are exemplified. A⁰″ is preferably an alkylene group having 1 to 5 carbon atoms or —O—.

The cyclic hydrocarbon group which may have a substituent for R1⁰¹ and R1⁰² may be a fused cyclic group containing a fused ring in which an aliphatic hydrocarbon ring and an aromatic ring are fused. Examples of fused rings include a ring in which one or more aromatic rings are fused to a polycycloalkane having a crosslinked ring type polycyclic framework. Specific examples of crosslinked ring type polycycloalkanes include bicycloalkanes such as bicyclo[2.2.1]heptane (norbornane), and bicyclo[2.2.2]octane. The fused cyclic type is preferably a group containing a fused ring in which 2 or 3 aromatic rings are fused to a bicycloalkane.

The cyclic hydrocarbon group which may have a substituent for R1⁰¹ and R1⁰² may be a group in which two or more ring structures selected from the group consisting of aromatic rings and aliphatic rings are linked. The two or more ring structures may be linked by a single bond or a divalent linking group. Examples of divalent linking groups include the same as those described above. The aromatic ring may be an aromatic hydrocarbon ring or an aromatic heterocycle. The aliphatic ring may be an aliphatic hydrocarbon ring or an aliphatic heterocycle. Examples of aromatic rings and aliphatic rings include the same as those described above. Examples of groups in which two or more ring structures are linked include a group in which two hydrogen atoms have been removed from biphenyl, a group in which two hydrogen atoms have been removed from bicyclohexyl, a group in which two hydrogen atoms have been removed from cyclohexylbenzene, and a group in which two hydrogen atoms have been removed from 2,2-diphenyl-1,3-oxolane.

Among the above examples, in order to improve uniformity within the resist film, R1⁰¹ and R1⁰² are preferably an aromatic hydrocarbon group which may have a substituent or a polycyclic alicyclic hydrocarbon group which may have a substituent, and more preferably an aromatic hydrocarbon group, a group in which two hydrogen atoms have been removed from a polycycloalkane or polycycloalkene, a lactone-containing cyclic group represented by General Formula (L-r-2), a fused cyclic group containing a fused ring in which an aliphatic hydrocarbon ring and an aromatic ring are fused, or a group in which two or more ring structures are linked.

R1⁰¹ and R1⁰² are preferably a group represented by any of the following Chemical Formulae (R10-1) to (R10-6), more preferably a group represented by the following Chemical Formula (R10-1), (R10-2), or (R10-5), still more preferably a group represented by the following Chemical Formula (R10-2) or (R10-5), and particularly preferably a group represented by the following Chemical Formula (R10-5).

The hydrogen atoms of the groups represented by the following Chemical Formulae (R10-1) to (R10-6) may each independently be substituted with a substituent. Examples of substituents include the same substituents as for Ra^{x5}.

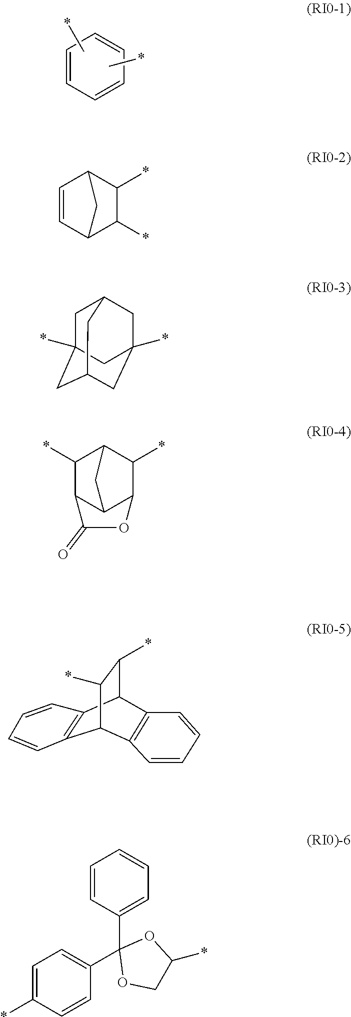

In General Formula (b0), L⁰¹ is a divalent linking group or a single bond, and L⁰² and L⁰³ are a divalent linking group.

Preferable examples of divalent linking groups for L⁰¹, L⁰² and L⁰³ include an oxygen atom-containing divalent linking group.

When L⁰¹, L⁰² and L⁰³ are an oxygen atom-containing divalent linking group, L⁰¹, L⁰² and L⁰³ may contain an atom other than an oxygen atom. Examples of atoms other than an oxygen atom include a carbon atom, a hydrogen atom, a sulfur atom, and a nitrogen atom.

Examples of oxygen atom-containing divalent linking groups include non-hydrocarbon-based oxygen atom-containing linking groups such as an oxygen atom (ether bond: —O—), an ester bond (—C(═O)—O—), an oxycarbonyl group (—O—C(═O)—), an amide bond (—C(═O)—NH—), a carbonyl group (—C(═O)—), and a carbonate bond (—O—C(═O)—O—); and a combination of the non-hydrocarbon-based oxygen atom-containing linking group and an alkylene group. A sulfonyl group (—SO₂—) may be additionally linked to this combination.

More specific examples of divalent linking groups for L⁰″, L⁰² and L⁰³ include —O—, —CO—, —OCO—, —COO—, —OCO—CH₂—O—, —SO₂—, —N(R^{a})—C(═O)—, —N(R^{a})—, —C(R^{a})(R^{a})—N(R^{a})—, —C(R^{a})(N(R^{a})(R^{a}))—, and —C(═O)—N(R^{a})—. R^{a}'s are each independently a hydrogen atom or an alkyl group.

L⁰¹ is preferably a divalent linking group, more preferably an oxygen atom-containing divalent linking group, still more preferably —OCO—, —COO—, —OCO—CH₂—O—, —C(═O)—N(R^{a})—, or —N(R^{a})—C(═O)—, and particularly preferably —OCO— or —COO—.

L⁰² and L⁰³ are more preferably an oxygen atom-containing divalent linking group, still more preferably —OCO—, —COO—, —OCO—CH₂—O—, —C(═O)—N(R^{a})—, or —N(R^{a})—C(═O)—, and particularly preferably —OCO— or —COO—.

In General Formula (b0), examples of substituents other than an iodine atom for R^{m1} include a hydroxy group, an alkyl group, a fluorinated alkyl group, a fluorine atom, and a chlorine atom.

The alkyl group and the alkyl group in the fluorinated alkyl group are preferably an alkyl group having 1 to 5 carbon atoms, and more preferably a methyl group or an ethyl group.

In General Formula (b0), the alkylene group and fluorinated alkylene group for Vb⁰ each preferably have 1 to 4 carbon atoms and more preferably have 1 to 3 carbon atoms.

Examples of fluorinated alkylene groups for Vb⁰ include a group in which some or all of the hydrogen atoms in the alkylene group are substituted with fluorine atoms.

Vb⁰ is preferably an alkylene group or a fluorinated alkylene group, more preferably an alkylene group having 1 to 4 carbon atoms or a fluorinated alkylene group having 1 to 4 carbon atoms, and still more preferably a methylene group, —CH(CF₃)—, —CH₂CH₂CF₂—, or —CH₂CH₂CHF—.

In General Formula (b0), R⁰ is preferably a fluorine atom or a perfluoroalkyl group having 1 to 5 carbon atoms, and more preferably a fluorine atom.

In General Formula (b0), nb1 is the number of iodine atoms (I).

nb1 is preferably an integer of 1 to 3, and more preferably 2 or 3.

In General Formula (b0), nb2 is preferably 1 or 2, and more preferably 1.

In General Formula (b0), nb3 is preferably 0 or 1, and more preferably 0.

nb1+nb2+nb3 is preferably 2 to 5, more preferably 2 to 4, and still more preferably 3 or 4.

In General Formula (b0), regarding mb1 and mb2, when mb1 is 1, mb2 is preferably 0, and when mb1 is 0, mb2 is preferably 1.

The component (B0) is preferably a compound represented by the following General Formula (b0-1) or (b0-2).

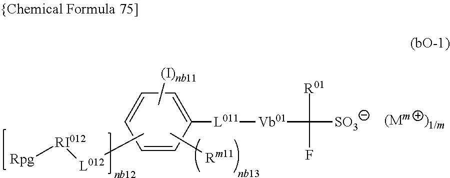

[in the formula, Rpg is an acid-decomposable group, R1⁰¹² is a cyclic hydrocarbon group which may have a substituent, L⁰¹¹ is a divalent linking group or a single bond, L⁰¹² is a divalent linking group, R^{m11} is a substituent other than an iodine atom, Vb⁰¹ is a single bond, an alkylene group or a fluorinated alkylene group, R⁰¹ is a hydrogen atom, a fluorinated alkyl group having 1 to 5 carbon atoms or a fluorine atom, nb11 is an integer of 1 to 4, nb12 is an integer of 1 to 4, nb13 is an integer of 0 to 3, and nb11+nb12+nb13≤5, M^{m+} is an m-valent organic cation, and m is an integer of 1 or more].

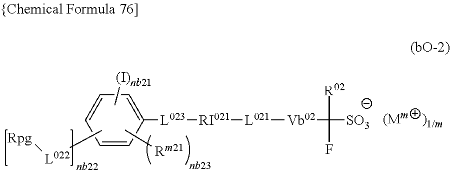

[in the formula, Rpg is an acid-decomposable group, R1⁰²¹ is a cyclic hydrocarbon group which may have a substituent, L⁰²¹ is a divalent linking group or a single bond, L⁰²² and L⁰²³ are each independently a divalent linking group, R^{m21} is a substituent other than an iodine atom, Vb⁰² is a single bond, an alkylene group or a fluorinated alkylene group, R⁰² is a hydrogen atom, a fluorinated alkyl group having 1 to 5 carbon atoms or a fluorine atom, nb21 is an integer of 1 to 4, nb22 is an integer of 1 to 4, nb23 is an integer of 0 to 3, and nb21+nb22+nb23≤5, M^{m+} is an m-valent organic cation, and m is an integer of 1 or more].

Rpg in Formulae (b0-1) and (b0-2) is the same as Rpg in Formula (b0).

Examples of R1⁰¹² and R1⁰²¹ in Formulae (b0-1) and (b0-2) include the same as R1⁰¹ and R1⁰² in Formula (b0), and a group represented by any of Chemical Formulae (R10-1) to (R10-6) is preferable, a group represented by Chemical Formula (R10-1), (R10-2), or (R10-5) is more preferable, a group represented by Chemical Formula (R10-2) or (R10-5) is still more preferable, and a group represented by Chemical Formula (R10-5) is particularly preferable.

Examples of divalent linking groups for L⁰¹¹, L¹², L⁰²¹, L⁰²², and L⁰²³ in Formulae (b0-1) and (b0-2) include the same groups for L⁰¹, L⁰², and L⁰³ in Formula (b0), and —OCO—, —COO—, or —C(═O)—N(R^{a})— is preferable, and —OCO— or —COO— is more preferable.

Examples of R^{m11} and R ^{m21} in Formulae (b0-1) and (b0-2) include the same as R^{m1} in Formula (b0), and an alkyl group having 1 to 5 carbon atoms is preferable, and a methyl group or an ethyl group is more preferable.

Examples of Vb⁰¹ and Vb⁰² in Formulae (b0-1) and (b0-2) include the same as Vb⁰ in Formula (b0), and an alkylene group having 1 to 4 carbon atoms or a fluorinated alkylene group having 1 to 4 carbon atoms is preferable, and a methylene group, —CH(CF₃)—, —CH₂CH₂CF₂—, or —CH₂CH₂CHF— is more preferable.

Examples of R⁰¹ and R⁰² in Formulae (b0-1) and (b0-2) include the same as R⁰ in Formula (b0), and a fluorine atom or a perfluoroalkyl group having 1 to 5 carbon atoms is preferable, and a fluorine atom is more preferable.

In Formulae (b0-1) and (b0-2), nb11 and nb21 are preferably an integer of 1 to 3, and more preferably 2 or 3.

In Formulae (b0-1) and (b0-2), nb12 and nb22 are preferably 1 or 2, and more preferably 1.

In Formulae (b0-1) and (b0-2), nb13 and nb23 are preferably 0 or 1 and more preferably 0.

Specific examples of components (B0) containing a preferable anion moiety are shown below, but the present invention is not limited thereto. In the following formulae, m is an integer of 1 or more, and M^{m+} is an m-valent organic cation.

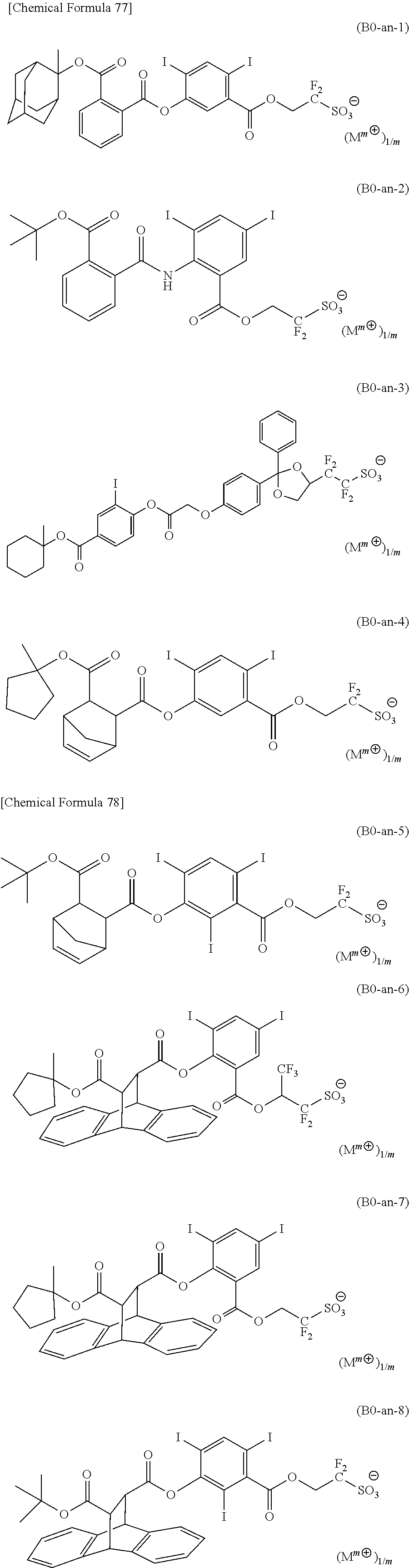

### [Cation Moiety]

In General Formula (b0), M^{m+} is an m-valent organic cation. The organic cation is preferably a sulfonium cation or an iodonium cation.

Preferable examples of cation moieties ((M^{m+})_{1/m}) include organic cations represented by General Formulae (ca-1) to (ca-3).

The cation moiety ((M′^{m+})_{1/m}) is preferably a sulfonium cation, more preferably a cation represented by Formula (ca-1), and particularly preferably a cation represented by each of Formulae (ca-1-1) to (ca-1-84).

Particularly, in order to achieve high sensitivity, as the preferable cation represented by Formula (ca-1), one having an electron-withdrawing group such as a fluorine atom, a fluorinated alkyl group, or a sulfonyl group, as a substituent, is preferable, and for example, a cation represented by each of Chemical Formulae (ca-1-44) and (ca-1-71) to (ca-1-84) is more preferable, and a cation represented by each of Chemical Formulae (ca-1-71) to (ca-1-75) and (ca-1-80) is still more preferable.

Preferable specific examples of components (B0) are shown below, but the present invention is not limited thereto.

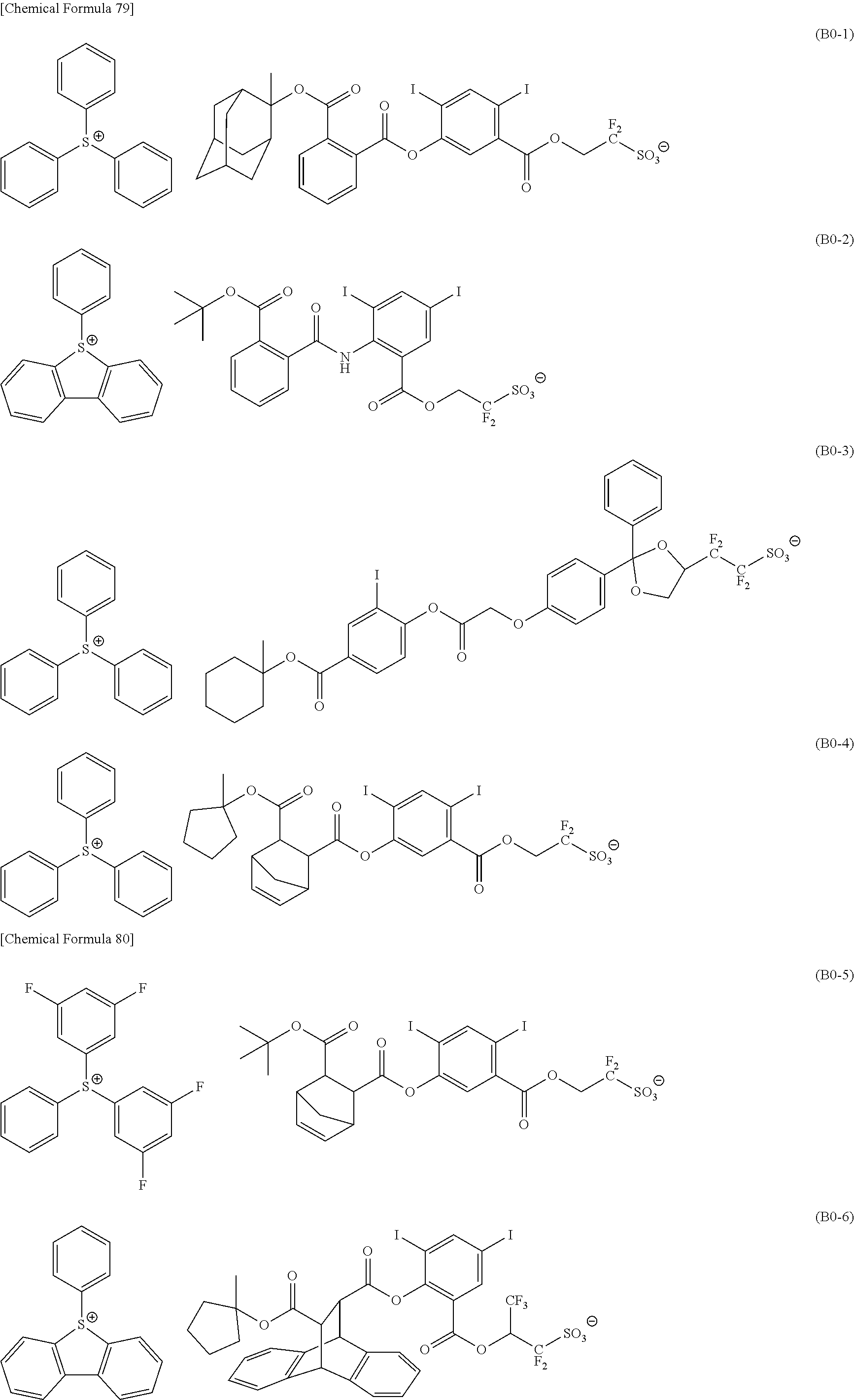

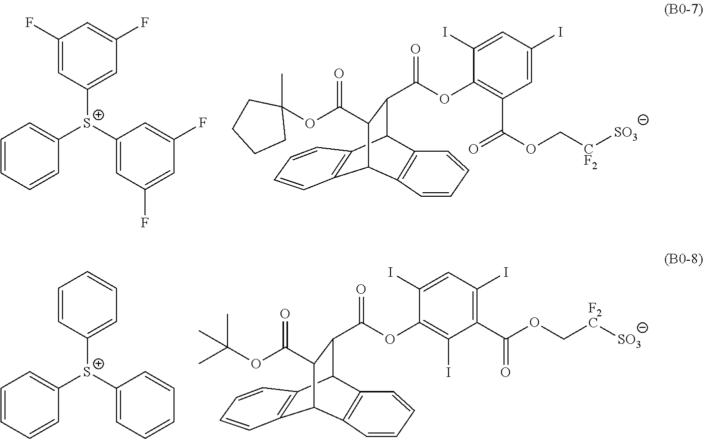

In the resist composition of the present embodiment, the components (B0) may be used alone or two or more thereof may be used in combination.

In the resist composition of the present embodiment, the content of the component (B0 with respect to 100 parts by mass of the component (A) is preferably 5 to 40 parts by mass, more preferably 10 to 35 parts by mass, and still more preferably 12 to 30 parts by mass.

When the content of the component (B0) is set to be equal to or larger than the lower limit value of the preferable range, in resist pattern formation, lithography properties such as sensitivity, CDU, and resolution are further improved. On the other hand, if the content of the component (B0) is set to be equal to or smaller than the upper limit value of the preferable range, when each component of the resist composition is dissolved in an organic solvent, a uniform solution is easily obtained, and the storage stability of the resist composition is further improved.

In the resist composition of the present embodiment, the proportion of the component (B0) in the component (B) with respect to a total mass of the component (B) is, for example, 50 mass % or more, preferably 70 mass % or more, and more preferably 95 mass % or more. The proportion of the component (B0) in the component (B) may be 100 mass %.

### <<Component (B1)>>

The component (B) may contain an acid generator component (B1) (hereinafter referred to as a “component (B1)”) other than the above component (B0).

The component (B1) is not particularly limited, and any of components that have been previously proposed as acid generators for chemically amplified resist compositions can be used.

Examples of such acid generators include onium salt-based acid generators such as iodonium salts and sulfonium salts, and oxime sulfonate-based acid generators; diazomethane-based acid generators such as bisalkyl or bisarylsulfonyldiazomethanes, and poly(bissulfonyl)diazomethanes; and various types such as nitrobenzyl sulfonate-based acid generators, imino sulfonate-based acid generators, and disulfone-based acid generators. The component (B) may be contained in the form of a compound or in a form that is incorporated as the above structural unit (a5) into the component (A1), or a combination of both forms.

Examples of onium salt-based acid generators include a compound represented by the following General Formula (b-1) (hereinafter referred to as a “component (b-1)”), a compound represented by General Formula (b-2) (hereinafter referred to as a “component (b-2)”) and a compound represented by General Formula (b-3) (hereinafter referred to as a “component (b-3)”).

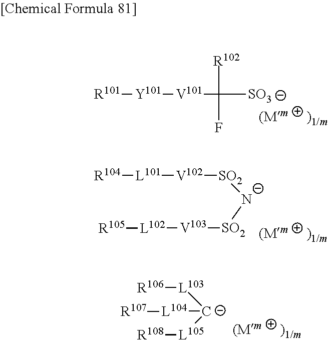

[in the formula, R¹⁰¹ and R¹⁰⁴ to R¹⁰⁸ are each independently a cyclic group which may have a substituent, a chain alkyl group which may have a substituent, or a chain alkenyl group which may have a substituent, R¹⁰⁴ and R¹⁰⁵ may be bonded to each other to form a ring structure, R¹⁰² is a fluorinated alkyl group having 1 to 5 carbon atoms or a fluorine atom, Y¹⁰¹ is an oxygen atom-containing divalent linking group or a single bond, V¹⁰¹ to V¹⁰³ are each independently a single bond, an alkylene group or a fluorinated alkylene group, where Y¹⁰¹ and V¹⁰¹ are not both a single bond, L¹⁰¹ to L¹⁰² are each independently a single bond or an oxygen atom, L¹⁰³ to L¹⁰⁵ are each independently a single bond, —CO— or —SO₂—, m is an integer of 1 or more, and M′^{m+} is an m-valent onium cation].

### {Anion Moiety}

Anion in Component (b-1)

In Formula (b-1), R¹⁰¹ is a cyclic group which may have a substituent, a chain alkyl group which may have a substituent, or a chain alkenyl group which may have a substituent.

Cyclic group which may have substituent:

The cyclic group is preferably a cyclic hydrocarbon group, and the cyclic hydrocarbon group may be an aromatic hydrocarbon group or an aliphatic hydrocarbon group. The aliphatic hydrocarbon group is a hydrocarbon group having no aromaticity. In addition, the aliphatic hydrocarbon group is preferably saturated.

The aromatic hydrocarbon group for R¹⁰¹ is a hydrocarbon group having an aromatic ring. The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 10 carbon atoms. Here, the number of carbon atoms does not include the number of carbon atoms in the substituent.

Specific examples of aromatic rings in the aromatic hydrocarbon group for R¹⁰¹ include benzene, fluorene, naphthalene, anthracene, phenanthrene, biphenyl and aromatic heterocycles in which some carbon atoms constituting these aromatic rings are substituted with heteroatoms. Examples of heteroatoms in aromatic heterocycles include an oxygen atom, a sulfur atom, and a nitrogen atom.

Specific examples of aromatic hydrocarbon groups for R¹⁰¹ include a group in which one hydrogen atom has been removed from the aromatic ring (an aryl group: for example, a phenyl group and a naphthyl group, etc.), and a group in which one hydrogen atom in the aromatic ring is substituted with an alkylene group (for example, a benzyl group, a phenethyl group, a 1-naphthyl methyl group, etc.). The alkylene group (the alkyl chain in the arylalkyl group) preferably has 1 to 4 carbon atoms, more preferably has 1 or 2 carbon atoms, and particularly preferably has 1 carbon atom.

Examples of cyclic aliphatic hydrocarbon groups for R¹⁰¹ include an aliphatic hydrocarbon group containing a ring in the structure.

Examples of aliphatic hydrocarbon groups containing a ring in the structure include an alicyclic hydrocarbon group (a group in which one hydrogen atom has been removed from an aliphatic hydrocarbon ring), a group in which an alicyclic hydrocarbon group is bonded to the end of a linear or branched aliphatic hydrocarbon group, and a group in which an alicyclic hydrocarbon group is inserted into a linear or branched aliphatic hydrocarbon group.

The alicyclic hydrocarbon group preferably has 3 to 20 carbon atoms and more preferably has 3 to 12 carbon atoms.

The alicyclic hydrocarbon group may be a polycyclic group or a monocyclic group. The monocyclic alicyclic hydrocarbon group is preferably a group in which one or more hydrogen atoms have been removed from a monocycloalkane. The monocycloalkane preferably has 3 to 6 carbon atoms, and specific examples thereof include cyclopentane and cyclohexane. The polycyclic alicyclic hydrocarbon group is preferably a group in which one or more hydrogen atoms have been removed from a polycycloalkane, and the polycycloalkane is preferably one having 7 to 30 carbon atoms. Among these, the polycycloalkane is more preferably a polycycloalkane having a crosslinked ring type polycyclic framework such as adamantane, norbornane, isobornane, tricyclo[5.2.1.02, 6]decane, and tetracyclododecane; or a polycycloalkane having a fused ring type polycyclic framework such as a cyclic group having a steroid framework.

Among these, the cyclic aliphatic hydrocarbon group for R¹⁰¹ is preferably a group in which one or more hydrogen atoms have been removed from a monocycloalkane or polycycloalkane, more preferably a group in which one hydrogen atom has been removed from a polycycloalkane, still more preferably an adamantyl group or a norbornyl group, and particularly preferably an adamantyl group.

The linear aliphatic hydrocarbon group which may be bonded to an alicyclic hydrocarbon group preferably has 1 to 10 carbon atoms, more preferably has 1 to 6 carbon atoms, still more preferably has 1 to 4 carbon atoms, and most preferably has 1 to 3 carbon atoms. The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group which may be bonded to an alicyclic hydrocarbon group preferably has 2 to 10 carbon atoms, more preferably has 3 to 6 carbon atoms, still more preferably has 3 or 4 carbon atoms, and most preferably has 3 carbon atoms. The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

In addition, the cyclic hydrocarbon group for R¹⁰¹ may contain a heteroatom such as in a heterocycle. Specific examples thereof include lactone-containing cyclic groups represented by General Formulae (a2-r-1) to (a2-r-7), —SO₂-containing cyclic groups represented by General Formulae (b5-r-1) to (b5-r-4), and other heterocyclic groups represented by chemical formulae (r-hr-1) to (r-hr-16).

Examples of substituents for the cyclic group for R¹⁰¹ include an alkyl group, an alkoxy group, a halogen atom, a halogenated alkyl group, a hydroxy group, a carbonyl group, and a nitro group.

The alkyl group as a substituent is preferably an alkyl group having 1 to 5 carbon atoms.

The alkoxy group as a substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

The halogen atom as a substituent is preferably a fluorine atom, a bromine atom, or an iodine atom.

Examples of halogenated alkyl groups as a substituent include an alkyl group having 1 to 5 carbon atoms, for example, a group in which some or all of the hydrogen atoms are substituted with halogen atoms such as a methyl group, an ethyl group, a propyl group, an n-butyl group, and a tert-butyl group.

The carbonyl group as a substituent is a group that substitutes a methylene group (—CH₂—) constituting a cyclic hydrocarbon group.

The cyclic hydrocarbon group for R¹⁰¹ may be a fused cyclic group containing a fused ring in which an aliphatic hydrocarbon ring and an aromatic ring are fused. Examples of fused rings include a ring in which one or more aromatic rings are fused to a polycycloalkane having a crosslinked ring type polycyclic framework. Specific examples of crosslinked ring type polycycloalkanes include bicycloalkanes such as bicyclo[2.2.1]heptane (norbornane), and bicyclo[2.2.2]octane. The fused cyclic type is preferably a group containing a fused ring in which 2 or 3 aromatic rings are fused to a bicycloalkane, and more preferably a group containing a fused ring in which 2 or 3 aromatic rings are fused to bicyclo[2.2.2]octane. Specific examples of fused cyclic groups for R¹⁰¹ include groups represented by the following Formulae (r-br-1) and (r-br-2). In the formulae, * indicates a bond boned to Y¹⁰¹ in Formula (b-1).

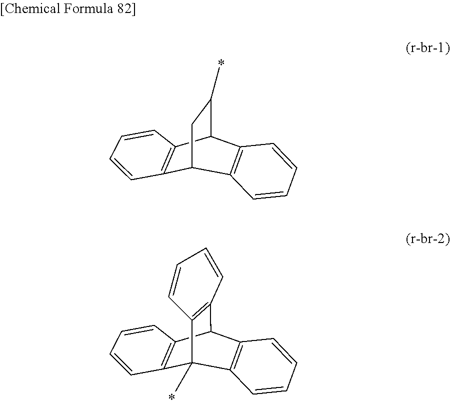

Examples of substituents that the fused cyclic group for R¹⁰¹ may have include an alkyl group, an alkoxy group, a halogen atom, a halogenated alkyl group, a hydroxy group, a carbonyl group, a nitro group, an aromatic hydrocarbon group, and an alicyclic hydrocarbon group.

Examples of alkyl groups, alkoxy groups, halogen atoms, and halogenated alkyl groups as substituents for the fused cyclic groups include the same as those exemplified as the substituents for the cyclic groups for R¹⁰¹.

Examples of aromatic hydrocarbon groups as a substituent for the fused cyclic group include a group in which one hydrogen atom has been removed from an aromatic ring (an aryl group: for example, a phenyl group, a naphthyl group, etc.), a group in which one hydrogen atom in the aromatic ring is substituted with an alkylene group (for example, arylalkyl groups such as a benzyl group, a phenethyl group, a 1-naphthyl methyl group, a 2-naphthyl methyl group, a 1-naphthyl ethyl group, and a 2-naphthyl ethyl group), and heterocyclic groups represented by Formulae (r-hr-1) to (r-hr-6).

Examples of alicyclic hydrocarbon groups as a substituent for the fused cyclic group include a group in which one hydrogen atom has been removed from a monocycloalkane such as cyclopentane and cyclohexane; a group in which one hydrogen atom has been removed from a polycycloalkane such as adamantane, norbornane, isobornane, tricyclo[5.2.1.0^{2,6}]decane, and tetracyclododecane; lactone-containing cyclic groups represented by General Formulae (a2-r-1) to (a2-r-7); —SO₂-containing cyclic groups represented by General Formulae (b5-r-1) to (b5-r-4); and heterocyclic groups represented by Formulae (r-hr-7) to (r-hr-16).

Chain alkyl group which may have substituent:

The chain alkyl group for R¹⁰¹ may be either linear or branched.

The linear alkyl group preferably has 1 to 20 carbon atoms, more preferably has 1 to 15 carbon atoms, and most preferably has 1 to 10 carbon atoms.

The branched alkyl group preferably has 3 to 20 carbon atoms, more preferably has 3 to 15 carbon atoms, and most preferably has 3 to 10 carbon atoms. Specific examples thereof include a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

Chain alkenyl group which may have substituent:

The chain alkenyl group for R¹⁰¹ may be either linear or branched, and preferably has 2 to 10 carbon atoms, more preferably has 2 to 5 carbon atoms, still more preferably has 2 to 4 carbon atoms, and particularly preferably has 3 carbon atoms. Examples of linear alkenyl groups include a vinyl group, a propenyl group (allyl group), and a butenyl group. Examples of branched alkenyl groups include a 1-methylvinyl group, a 2-methylvinyl group, a 1-methylpropenyl group, and a 2-methylpropenyl group.

Among the above examples, the chain alkenyl group is preferably a linear alkenyl group, more preferably a vinyl group or a propenyl group, and particularly preferably a vinyl group.

Examples of substituents in the chain alkyl group or alkenyl group for R¹⁰¹ include an alkoxy group, a halogen atom, a halogenated alkyl group, a hydroxy group, a carbonyl group, a nitro group, an amino group, and the cyclic groups for R¹⁰¹.

In Formula (b-1), Y¹⁰¹ is a single bond or an oxygen atom-containing divalent linking group.

When Y¹⁰¹ is an oxygen atom-containing divalent linking group, Y¹⁰¹ may contain an atom other than an oxygen atom. Examples of atoms other than an oxygen atom include a carbon atom, a hydrogen atom, a sulfur atom, and a nitrogen atom.

Examples of oxygen atom-containing divalent linking groups include linking groups represented by General Formulae (L-a1-1) to (L-al-8). Here, in the following General Formulae (L-al-1) to (L-al-8), one bonded to R¹⁰¹ in Formula (b-1) is V′¹⁰¹ in the following General Formulae (L-al-1) to (L-al-8).

In Formula (b-1), V¹⁰¹ is a single bond, an alkylene group or a fluorinated alkylene group. Among these, V¹⁰¹ is preferably a single bond or a linear fluorinated alkylene group having 1 to 4 carbon atoms.

In Formula (b-1), R¹⁰² is a fluorine atom or a fluorinated alkyl group having 1 to 5 carbon atoms. R¹⁰² is preferably a fluorine atom or a perfluoroalkyl group having 1 to 5 carbon atoms, and more preferably a fluorine atom.

Specific examples of anion moieties represented by Formula (b-1) include fluorinated alkylsulfonate anions such as a trifluoromethanesulfonate anion and a perfluorobutanesulfonate anion, for example, when Y¹⁰¹ is a single bond; and anions represented by any of the following Formulae (an-1) to (an-3) when Y¹⁰¹ is an oxygen atom-containing divalent linking group.

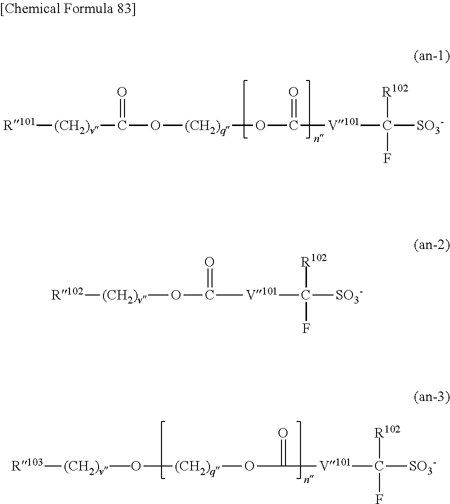

[in the formula, R″¹⁰¹ is an aliphatic cyclic group which may have a substituent, a monovalent heterocyclic group represented by each of Chemical Formulae (r-hr-1) to (r-hr-16), a fused cyclic group represented by Formula (r-br-1) or (r-br-2), a chain alkyl group which may have a substituent or an aromatic cyclic group which may have a substituent, R″¹⁰² is an aliphatic cyclic group which may have a substituent, a fused cyclic group represented by Formula (r-br-1) or (r-br-2), a lactone-containing cyclic group represented by each of General Formulae (a2-r-1), and (a2-r-3) to (a2-r-7), or a —SO₂-containing cyclic group represented by each of General Formulae (b5-r-1) to (b5-r-4), R″¹⁰³ is an aromatic cyclic group which may have a substituent, an aliphatic cyclic group which may have a substituent, or a chain alkenyl group which may have a substituent, V″101 is a single bond, an alkylene group having 1 to 4 carbon atoms, or a fluorinated alkylene group having 1 to 4 carbon atoms, R¹⁰² is a fluorine atom or a fluorinated alkyl group having 1 to 5 carbon atoms, v″'s are each independently an integer of 0 to 3, q″'s are each independently an integer of 0 to 20, and n″ is 0 or 1].

The aliphatic cyclic group which may have a substituent for R″¹⁰¹, R″¹⁰² and R″¹⁰³ is preferably a group exemplified as the cyclic aliphatic hydrocarbon group for R101 in Formula (b-1). Examples of substituents include the same substituents that may substitute the cyclic aliphatic hydrocarbon group for R¹⁰¹ in Formula (b-1).

The aromatic cyclic group which may have a substituent for R″¹⁰¹ and R″¹⁰³ is preferably a group exemplified as the aromatic hydrocarbon group in the cyclic hydrocarbon group for R¹⁰¹ in Formula (b-1). Examples of substituents include the same substituents that may substitute the aromatic hydrocarbon group for R¹⁰¹ in Formula (b-1).

The chain alkyl group which may have a substituent for R″¹⁰¹ is preferably a group exemplified as the chain alkyl group for R¹⁰¹ in Formula (b-1).

The chain alkenyl group which may have a substituent for R″¹⁰³ is preferably a group exemplified as the chain alkenyl group for R¹⁰¹ in Formula (b-1).

—Anion in Component (b-2)

In Formula (b-2), R¹⁰⁴ and R¹⁰⁵ are each independently a cyclic group which may have a substituent, a chain alkyl group which may have a substituent, or a chain alkenyl group which may have a substituent, and examples thereof include the same groups as for R¹⁰¹ in Formula (b-1). Here, R¹⁰⁴ and R¹⁰⁵ may be bonded to each other to form a ring.

R¹⁰⁴ and R¹⁰⁵ are preferably a chain alkyl group which may have a substituent and more preferably a linear or branched alkyl group or a linear or branched fluorinated alkyl group.

The chain alkyl group preferably has 1 to 10 carbon atoms, more preferably has 1 to 7 carbon atoms, and still more preferably has 1 to 3 carbon atoms. The number of carbon atoms in the chain alkyl group for R¹⁰⁴ and R¹⁰⁵ is preferably a smaller number within the above range of the number of carbon atoms, for reasons such as favorable solubility in a resist solvent. In addition, in the chain alkyl group for R¹⁰⁴ and R¹⁰⁵, a larger number of hydrogen atoms that are substituted with fluorine atoms is preferable because the strength of the acid is stronger and the transparency to high-energy light of 250 nm or less and electron beams is improved. The proportion of fluorine atoms in the chain alkyl group, that is, the fluorination rate, is preferably 70 to 100%, and more preferably 90 to 100%, and a perfluoroalkyl group in which all hydrogen atoms are substituted with fluorine atoms is most preferable.

In Formula (b-2), V¹⁰² and V¹⁰³ are each independently a single bond, an alkylene group, or a fluorinated alkylene group, and examples thereof include the same groups as for V¹⁰¹ in Formula (b-1).

In Formula (b-2), L¹⁰¹ and L¹⁰² are each independently a single bond or an oxygen atom.

—Anion in Component (b-3)

In Formula (b-3), R¹⁰⁶ to R¹⁰⁸ are each independently a cyclic group which may have a substituent, a chain alkyl group which may have a substituent, or a chain alkenyl group which may have a substituent, and examples thereof include the same groups as for R¹⁰¹ in Formula (b-1).

In Formula (b-3), L¹⁰³ to L¹⁰⁵ are each independently a single bond, —CO— or —SO₂—.

Among the above examples, the anion moiety of the component (B1) is preferably an anion in the component (b-1) and more preferably an anion represented by Formula (an-1).

### {Cation Moiety}

In Formula (b-1), Formula (b-2), and Formula (b-3), M′^{m+} is an m-valent onium cation. Among these, a sulfonium cation and an iodonium cation are preferable. m is an integer of 1 or more.

The cation moiety of the component (B1) is preferably a sulfonium cation, more preferably a cation represented by each of Formulae (ca-1) to (ca-3), still more preferably a cation represented by Formula (ca-1), and particularly preferably a cation represented by each of Formulae (ca-1-1) to (ca-1-83).

In the resist composition of the present embodiment, the components (B1) may be used alone or two or more thereof may be used in combination.

The content of the component (B1) with respect to 100 parts by mass of the component (A) is preferably 20 parts by mass or less, more preferably 10 parts by mass or less, and still more preferably 5 parts by mass or less.

The component (B) preferably does not include the component (B1) and preferably includes only the component (B0).

### <Fluorine Additive Component (F)>

The resist composition of the present embodiment contains a fluorine additive component (F) (component (F)) that is decomposable in an alkaline development solution.

<<Structural Unit (f0)>>

The component (F) preferably includes a fluorine resin component (F0) having a structural unit (f0) represented by the following General Formula (f0) (hereinafter referred to as a “component (F0)”).

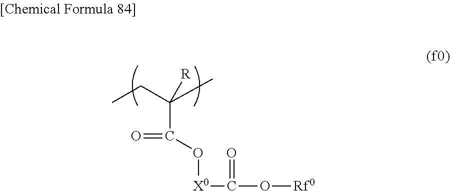

[in the formula, R is a hydrogen atom, an alkyl group having 1 to 5 carbon atoms or a halogenated alkyl group having 1 to 5 carbon atoms, X⁰ is a divalent linking group having no acid-dissociable moiety, and Rf⁰ is an organic group having a fluorine atom].

The structural unit (f0) is a structural unit having a base-dissociable group. The “base-dissociable group” is an organic group that is dissociable under the action of a base. That is, the “base-dissociable group” dissociates under the action of an alkaline development solution (for example, a 2.38 mass % aqueous TMAH solution at 23° C.). In Formula (f0), the group represented by Rf⁰ is a base-dissociable group. When the component (F0) has the structural unit (f0), it is decomposable in an alkaline development solution.

In General Formula (f0), the alkyl group having 1 to 5 carbon atoms for R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms for R is a group in which some or all of the hydrogen atoms in the alkyl group having 1 to 5 carbon atoms are substituted with halogen atoms. Examples of halogen atoms include a fluorine atom, a chlorine atom, a bromine atom, and an iodine atom, and a fluorine atom is particularly preferable.

R is preferably a hydrogen atom, an alkyl group having 1 to 5 carbon atoms or a fluorinated alkyl group having 1 to 5 carbon atoms, and in terms of industrial availability, a hydrogen atom, a methyl group or a trifluoromethyl group is more preferable, a hydrogen atom or a methyl group is still more preferable, and a methyl group is particularly preferable.

In General Formula (f0), X⁰ is a divalent linking group having no acid-dissociable moiety.

The “acid-dissociable moiety” is a moiety within the organic group such as the above “acid-dissociable group,” that dissociates under the action of an acid generated upon exposure. Since X⁰ does not contain an acid-dissociable moiety, an O atom of (—C(═O)—O—) bonded to the main chain in Formula (f0) and Rf⁰ do not dissociate under the action of an acid.

Examples of divalent linking groups having no acid-dissociable moiety for X⁰ include a divalent hydrocarbon group which may have a substituent and a heteroatom-containing divalent linking group.

Divalent hydrocarbon group which may have substituent:

The hydrocarbon group may be an aliphatic hydrocarbon group or an aromatic hydrocarbon group.

The aliphatic hydrocarbon group is a hydrocarbon group having no aromaticity. The aliphatic hydrocarbon group may be saturated or unsaturated, and is generally preferably saturated.

More specific examples of aliphatic hydrocarbon groups include a linear or branched aliphatic hydrocarbon group and an aliphatic hydrocarbon group containing a ring in the structure.

The linear or branched aliphatic hydrocarbon group preferably has 1 to 10 carbon atoms, more preferably has 1 to 8 carbon atoms, still more preferably has 1 to 5 carbon atoms, and particularly preferably has 1 to 4 carbon atoms.

The linear aliphatic hydrocarbon group is preferably a linear alkylene group, and specific examples thereof include a methylene group, an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group is preferably a branched alkylene group, and specific examples thereof include alkylalkylene groups, for example, alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyltrimethylene groups such as —CH(CH₃)CH₂CH₂— and —CH₂CH(CH₃)CH₂—; and alkyltetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The chain (linear or branched) aliphatic hydrocarbon group may or may not have a substituent. Examples of substituents include a fluorine atom, a fluorinated alkyl group having 1 to 5 carbon atoms and substituted with a fluorine atom, and an oxygen atom (═O).

Examples of aliphatic hydrocarbon groups containing a ring include a cyclic aliphatic hydrocarbon group (a group in which two hydrogen atoms have been removed from an aliphatic hydrocarbon ring), and a group in which the cyclic aliphatic hydrocarbon group is bonded to the end of the chain aliphatic hydrocarbon group or is inserted into a chain aliphatic hydrocarbon group.

The cyclic aliphatic hydrocarbon group preferably has 3 to 20 carbon atoms and more preferably has 3 to 12 carbon atoms.

The cyclic aliphatic hydrocarbon group may be a polycyclic group or a monocyclic group. The monocyclic group is preferably a group in which two hydrogen atoms have been removed from a monocycloalkane having 3 to 6 carbon atoms, and examples of monocycloalkanes include cyclopentane and cyclohexane.

The polycyclic group is preferably a group in which two hydrogen atoms have been removed from a polycycloalkane having 7 to 12 carbon atoms, and specific examples of polycycloalkanes include adamantane, norbornane, isobornane, tricyclodecane, and tetracyclododecane.

The cyclic aliphatic hydrocarbon group may or may not have a substituent. Examples of substituents include a lower alkyl group having 1 to 5 carbon atoms, a fluorine atom, a fluorinated alkyl group having 1 to 5 carbon atoms and substituted with a fluorine atom, and an oxygen atom (═O).

In the cyclic aliphatic hydrocarbon group, some carbon atoms constituting the ring structure may be substituted with a heteroatom-containing substituent. Examples of heteroatom-containing substituents include —O—, —C(═O)—O—, —S—, —S(═O)₂—, and —S(═O)₂—O—. Examples of cyclic aliphatic hydrocarbon groups containing heteroatoms in its ring framework include a lactone-containing cyclic group and a —SO₂— containing cyclic group. Examples of lactone-containing cyclic groups include groups represented by Formulae (L-r-1) to (L-r-7).

The aromatic hydrocarbon group for X⁰ may be monocyclic or polycyclic. The aromatic ring contained in the aromatic hydrocarbon group for X⁰ preferably has 5 to 30 carbon atoms, more preferably has 5 to 20 carbon atoms, still more preferably has 6 to 15 carbon atoms, and particularly preferably has 6 to 12 carbon atoms. Here, the number of carbon atoms does not include the number of carbon atoms in the substituent. Specific examples of aromatic rings include aromatic hydrocarbon rings such as benzene, naphthalene, anthracene, and phenanthrene; and aromatic heterocycles in which some carbon atoms constituting the aromatic hydrocarbon ring are substituted with heteroatoms. Examples of heteroatoms in aromatic heterocycles include an oxygen atom, a sulfur atom, and a nitrogen atom. Specific examples of aromatic heterocycles include a pyridine ring and a thiophene ring.

Examples of aromatic hydrocarbon groups for X⁰ include a group in which two hydrogen atoms have been removed from the aromatic hydrocarbon ring or aromatic heterocycle (an arylene group or a heteroarylene group); a group in which two hydrogen atoms have been removed from an aromatic compound containing two or more aromatic rings (for example, biphenyl, fluorene, etc.); and a group in which one hydrogen atom of a group (an aryl group or a heteroaryl group) in which one hydrogen atom has been removed from the aromatic hydrocarbon ring or aromatic heterocycle is substituted with an alkylene group (for example, a group in which one hydrogen atom has been additionally removed from the aryl group in the arylalkyl group such as a benzyl group, a phenethyl group, a 1-naphthyl methyl group, a 2-naphthyl methyl group, a 1-naphthyl ethyl group, and a 2-naphthyl ethyl group). The alkylene group bonded to the aryl group or heteroaryl group preferably has 1 to 4 carbon atoms, more preferably has 1 or 2 carbon atoms, and particularly preferably has 1 carbon atom.

The aromatic hydrocarbon group for X⁰ is preferably an arylene group or a heteroarylene group, and more preferably a phenylene group or a naphthyl group.

The aromatic hydrocarbon group for X⁰ may or may not have a substituent. Examples of substituents include a lower alkyl group having 1 to 5 carbon atoms, a fluorine atom, a fluorinated lower alkyl group having 1 to 5 carbon atoms and substituted with a fluorine atom, and an oxygen atom (═O).

The hydrocarbon group which may have a substituent for X⁰ is preferably a linear, branched or cyclic aliphatic hydrocarbon group, an arylene group, or a heteroarylene group, more preferably a linear or branched aliphatic hydrocarbon group, a lactone-containing cyclic group represented by any of Formulae (L-r-1) to (L-r-7) or a phenylene group, and still more preferably a linear or branched aliphatic hydrocarbon group. The linear or branched aliphatic hydrocarbon group may have a substituent. The linear or branched aliphatic hydrocarbon group is preferably a linear or branched alkylene group having 1 to 5 carbon atoms or a linear or branched fluorinated alkylene group having 1 to 5 carbon atoms.

### Heteroatom-Containing Divalent Linking Group

Examples of heteroatoms include an oxygen atom, a nitrogen atom, a sulfur atom, and a halogen atom.

Examples of heteroatom-containing divalent linking groups include combinations of a divalent hydrocarbon group with one or more selected from the group consisting of —O—, —C(═O)—, —C(═O)—O—, a carbonate bond (—O—C(═O)—O—), —NH—, —NR⁰⁴ (R⁰⁴ is an alkyl group)-, —NH—C(═O)—, and ═N—.

Examples of divalent hydrocarbon groups include the same groups described in the above hydrocarbon group which may have a substituent, and a linear or branched aliphatic hydrocarbon group or an aromatic hydrocarbon group is preferable.

The divalent linking group which is a combination of a heteroatom-containing divalent group and an aromatic hydrocarbon group is, for example, a group represented by **—(ArX)—O—RZ—*. In the formula, ArX is a divalent aromatic hydrocarbon group, RZ is a divalent linear or branched aliphatic hydrocarbon group, ** is a bond to (—O—) in Formula (f0), and * is a bond to a carbon atom of —(C═O)—O— in Formula (f0).

ArX is preferably a phenylene group.

RZ is preferably a divalent linear aliphatic hydrocarbon group having 1 to 5 carbon atoms, more preferably a divalent linear alkylene group having 1 to 3 carbon atoms, and still more preferably an ethylene group or a methylene group.

X⁰ is preferably a divalent hydrocarbon group which may have a substituent, preferably a linear, branched or cyclic aliphatic hydrocarbon group, an arylene group, or a heteroarylene group, more preferably a linear or aliphatic hydrocarbon group which may have a substituent, a lactone-containing cyclic group represented by any of Formulae (L-r-1) to (L-r-7), or a phenylene group, and still more preferably a linear or branched aliphatic hydrocarbon group which may have a substituent.

In order to improve sensitivity, resolution, and CDU, X⁰ is preferably a cyclic group other than a lactone-containing cyclic group or a linear or branched aliphatic hydrocarbon group which may have a substituent, and more preferably a linear or branched aliphatic hydrocarbon group which may have a substituent. In order to improve sensitivity, resolution, and CDU, X⁰ preferably does not contain a cyclic group. X⁰ is preferably a linear aliphatic hydrocarbon group having 1 to 5 carbon atoms which may have a substituent or a branched aliphatic hydrocarbon group having 3 to 5 carbon atoms which may have a substituent, more preferably a linear alkylene group having 1 to 5 carbon atoms which may have a substituent or a branched alkylene group having 3 to 5 carbon atoms which may have a substituent, and still more preferably a linear alkylene group having 1 to 5 carbon atoms, a branched alkylene group having 3 to 5 carbon atoms, a linear fluorinated alkylene group having 1 to 5 carbon atoms, or a branched fluorinated alkylene group having 3 to 5 carbon atoms.

In General Formula (f0), Rf⁰ is preferably a fluorine atom-containing hydrocarbon group.

The fluorine atom-containing hydrocarbon group may be linear, branched or cyclic, and preferably has 1 to 20 carbon atoms, more preferably has 1 to 15 carbon atoms, still more preferably has 1 to 10 carbon atoms, and particularly preferably has 1 to 5 carbon atoms.

In addition, the number of fluorine atoms in the fluorine atom-containing hydrocarbon group is preferably 1 to 10, more preferably 1 to 6, and still more preferably 1 to 3.

The fluorine atom-containing hydrocarbon group may be an aliphatic hydrocarbon group or an aromatic hydrocarbon group, and is preferably an aliphatic hydrocarbon group.

The aliphatic hydrocarbon group may be saturated or unsaturated, but is preferably saturated.

The aliphatic hydrocarbon group is preferably a linear or branched alkyl group having 1 to 20 carbon atoms, more preferably a linear or branched alkyl group having 1 to 15 carbon atoms, still more preferably a linear or branched alkyl group having 1 to 10 carbon atoms, and particularly preferably a linear or branched alkyl group having 1 to 5 carbon atoms.

The fluorine atom-containing hydrocarbon group is preferably a group in which some or all of the hydrogen atoms in the above hydrocarbon group are substituted with fluorine atoms.

In the fluorine atom-containing hydrocarbon group, it is preferable that 25% or more of hydrogen atoms in the hydrocarbon group be fluorinated and it is more preferable that 40% or more thereof be fluorinated.

Rf⁰ is preferably a fluorinated hydrocarbon group having 1 to 6 carbon atoms, more preferably a linear or branched fluorinated alkyl group having 1 to 6 carbon atoms, and still more preferably a trifluoromethyl group, —CH₂—CF₃, —CH₂—CH₂—CF₃, —CH₂—CF₂—CF₃, or —CH(CF₃)₂.

Preferable specific examples of structural units (f0) are shown below, but the present invention is not limited thereto.

In the following formulae, R^{α} is a hydrogen atom, a methyl group or a trifluoromethyl group.

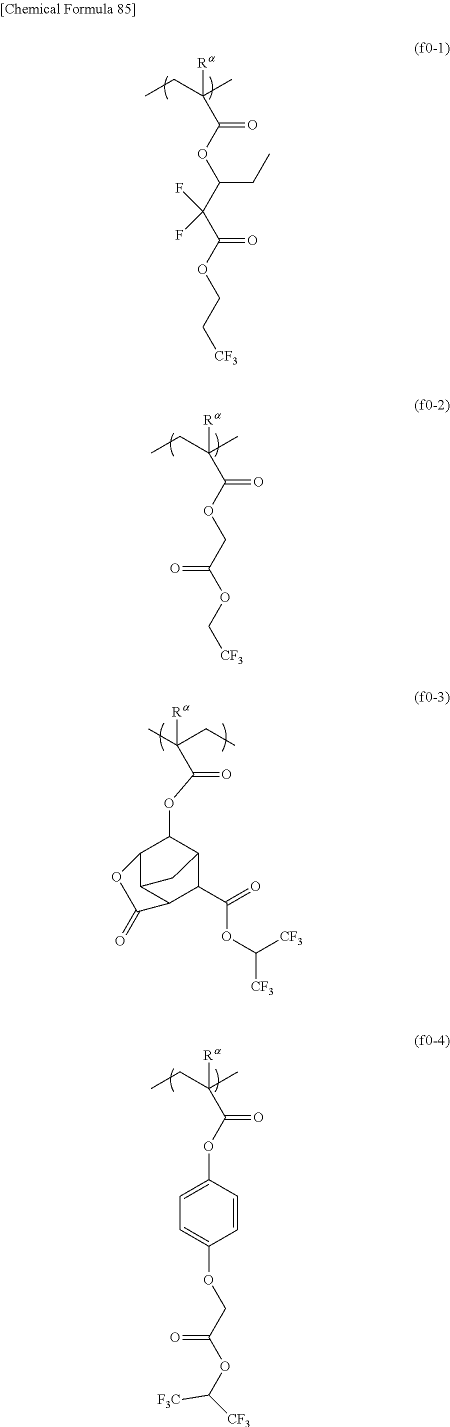

The structural unit (f0) of the component (F0) may be of one type or of two or more types.

The proportion of the structural unit (f0) in the component (F0) based on a total amount (100 mol %) of all structural units constituting the component (F0) is preferably 10 to 100 mol %, more preferably 30 to 100 mol %, still more preferably 40 to 100 mol %, and particularly preferably 50 to 100 mol %.

When the proportion of the structural unit (f0) is set to be equal to or larger than the lower limit value of the preferable range, lithography properties such as sensitivity, resolution, and CDU are improved.

### <<Other Structural Units>>

The component (F0) may have, in addition to the structural unit (f0), other structural units. The component (F0) may or may not have, but preferably has, a structural unit other than the structural unit (f0).

Examples of other structural units include the structural unit (al), the structural unit (a2), the structural unit (a8), a structural unit (f1) represented by the following General Formula (f1), and a structural unit (f2) represented by the following General Formula (f2) (excluding those that correspond to the structural unit (f0)).

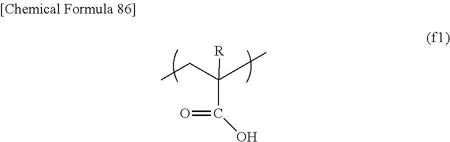

[in the formula, R is a hydrogen atom, an alkyl group having 1 to 5 carbon atoms or a halogenated alkyl group having 1 to 5 carbon atoms].

In Formula (f1), R is the same as R in Formula (f0). R is preferably a hydrogen atom, an alkyl group having 1 to 5 carbon atoms or a fluorinated alkyl group having 1 to 5 carbon atoms. In terms of industrial availability, R is more preferably a hydrogen atom, a methyl group or a trifluoromethyl group, still more preferably a hydrogen atom or a methyl group, and particularly preferably a methyl group.

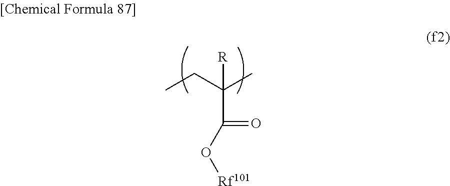

[in the formula, R is a hydrogen atom, an alkyl group having 1 to 5 carbon atoms or a halogenated alkyl group having 1 to 5 carbon atoms, and Rf¹⁰¹ is a fluorine atom-containing organic group].

In Formula (f2), R is the same as R in Formula (f0). R is preferably a hydrogen atom, an alkyl group having 1 to 5 carbon atoms or a fluorinated alkyl group having 1 to 5 carbon atoms. In terms of industrial availability, R is more preferably a hydrogen atom, a methyl group or a trifluoromethyl group, still more preferably a hydrogen atom or a methyl group, and particularly preferably a methyl group.

In Formula (f2), Rf¹⁰¹ is a fluorine atom-containing organic group, and preferably a fluorine atom-containing hydrocarbon group. Rf¹⁰¹ having no base-dissociable group is preferable.

The fluorine atom-containing hydrocarbon group may be linear, branched or cyclic, and preferably has 1 to 20 carbon atoms, more preferably has 1 to 15 carbon atoms, and particularly preferably has 1 to 10 carbon atoms.

The fluorine atom-containing hydrocarbon group may be an aliphatic hydrocarbon group or an aromatic hydrocarbon group, and is preferably an aliphatic hydrocarbon group.

The aliphatic hydrocarbon group may be saturated or unsaturated, but is preferably saturated.

The aliphatic hydrocarbon group is preferably a linear or branched alkyl group having 1 to 20 carbon atoms, more preferably a linear or branched alkyl group having 1 to 15 carbon atoms, and still more preferably a linear or branched alkyl group having 1 to 10 carbon atoms.

In the fluorine atom-containing hydrocarbon group, it is preferable that 25% or more of hydrogen atoms in the hydrocarbon group be fluorinated, it is more preferable that 30% or more thereof be fluorinated, and it is still more preferable that 40% or more thereof be fluorinated.

Rf¹⁰¹ is preferably a fluorinated hydrocarbon group having 1 to 10 carbon atoms, more preferably a linear or branched fluorinated alkyl group having 1 to 10 carbon atoms, and still more preferably a branched fluorinated alkyl group having 3 to 10 carbon atoms. Specific examples of Rf¹⁰¹ include a trifluoromethyl group, —CH₂—CF₃, —CH₂—CF₂—CF₃, —CH(CF₃)₂, —CH₂—CH₂—CF₃, —CH₂—CH₂—CF₂—CF₂—CF₂—CF₃, and —CH(CH₂—CH(CH₂)₂)(CF₂—CF₂—CF₃).

Examples of components (F0) include a polymer compound having the structural unit (f1); a polymer compound having the structural unit (f0) and the structural unit (a1); a polymer compound having the structural unit (f0) and the structural unit (a2); a polymer compound having the structural unit (f⁰) and the structural unit (f1); a polymer compound having the structural unit (f0) and the structural unit (f1); a polymer compound having the structural unit (f0), the structural unit (a1) and the structural unit (f2); and a polymer compound having the structural unit (f0), the structural unit (a1) and the structural unit (f8).

More specific examples of components (F0) include a polymer compound composed of the structural unit (f0); a polymer compound composed of the structural unit (f0) and the structural unit (al); a polymer compound composed of the structural unit (f0) and the structural unit (a2); a polymer compound composed of the structural unit (f0) and the structural unit (f1); a polymer compound composed of the structural unit (f0) and the structural unit (f2); a polymer compound composed of the structural unit (f0), the structural unit (a1) and the structural unit (f1); and a polymer compound composed of the structural unit (f0) and the structural unit (f8).

The structural unit (a1) preferably one containing an acid-dissociable group represented by Formula (al-r-2), more preferably one containing an acid-dissociable group represented by any of Formulae (a1-r2-1) to (al-r2-4), and still more preferably one containing an acid-dissociable group represented by Formula (a1-r2-1). The structural unit (a1) is preferably a structural unit represented by Formula (al-1) and more preferably a structural unit in which Ra¹ in Formula (a1-1) is an acid-dissociable group represented by Formula (a1-r-2).

The structural unit (a2) is preferably one containing a lactone-containing cyclic group represented by any of Formulae (a2-r-1) to (a2-r-7), more preferably one containing a lactone-containing cyclic group represented by Formula (a2-r-1), (a2-r-2), (a2-r-6) or (a2-r-7), and still more preferably one containing a lactone-containing cyclic group represented by Formula (a2-r-1), (a2-r-2), or (a2-r-6). The structural unit (a2) is preferably a structural unit represented by Formula (a2-1) and more preferably a structural unit in which Ra²¹ in Formula (a2-1) is a lactone-containing cyclic group represented by any of Formulae (a2-r-1) to (a2-r-7).

The structural unit (a8) is preferably a structural unit represented by any of Formulae (a8-1-01) to (a8-1-12) and more preferably a structural unit represented by Formula (a8-1-01).

In the polymer compound composed of a repeating structure of the structural unit (f0) and the structural unit (a1), the proportion of the structural unit (f0) based on a total amount (100 mol %) of all structural units constituting the polymer compound is preferably 40 to 95 mol %, more preferably 50 to 90 mol %, and still more preferably 60 to 80 mol %.

In addition, the proportion of the structural unit (al) in the polymer compound based on a total amount (100 mol %) of all structural units constituting the polymer compound is preferably 5 to 60 mol %, more preferably 10 to 50 mol %, and still more preferably 20 to 40 mol %.

In the polymer compound composed of a repeating structure of the structural unit (f0) and the structural unit (a2), the proportion of the structural unit (f0) based on a total amount (100 mol %) of all structural units constituting the polymer compound is preferably 40 to 95 mol %, more preferably 50 to 90 mol %, and still more preferably 60 to 80 mol %.

The proportion of the structural unit (a2) in the polymer compound based on a total amount (100 mol %) of all structural units constituting the polymer compound is preferably 5 to 60 mol %, more preferably 10 to 50 mol %, and still more preferably 20 to 40 mol %.

In the polymer compound composed of a repeating structure of the structural unit (f0) and the structural unit (f1), the proportion of the structural unit (f0) based on a total amount (100 mol %) of all structural units constituting the polymer compound is preferably 40 to 95 mol %, more preferably 50 to 90 mol %, and still more preferably 60 to 80 mol %.

The proportion of the structural unit (f1) in the polymer compound based on a total amount (100 mol %) of all structural units constituting the polymer compound is preferably 5 to 60 mol %, more preferably 10 to 50 mol %, and still more preferably 20 to 40 mol %.

In the polymer compound composed of a repeating structure of the structural unit (f0) and the structural unit (f2), the proportion of the structural unit (f0) based on a total amount (100 mol %) of all structural units constituting the polymer compound is preferably 30 to 95 mol %, more preferably 40 to 95 mol %, and still more preferably 50 to 80 mol %.

The proportion of the structural unit (f2) in the polymer compound based on a total amount (100 mol %) of all structural units constituting the polymer compound is preferably 5 to 60 mol %, more preferably 10 to 50 mol %, and still more preferably 20 to 40 mol %.

In the polymer compound composed of a repeating structure of the structural unit (f1), the structural unit (a1), and the structural unit (f1), the proportion of the structural unit (f0) based on a total amount (100 mol %) of all structural units constituting the polymer compound is preferably 40 to 95 mol %, more preferably 50 to 90 mol %, and still more preferably 60 to 80 mol %.

The proportion of the structural unit (a1) in the polymer compound based on a total amount (100 mol %) of all structural units constituting the polymer compound is preferably 1 to 50 mol %, more preferably 5 to 40 mol %, and still more preferably 10 to 30 mol %.

In addition, the proportion of the structural unit (f1) in the polymer compound based on a total amount (100 mol %) of all structural units constituting the polymer compound is preferably 1 to 40 mol %, more preferably 1 to 30 mol %, and still more preferably 5 to 20 mol %.

In the polymer compound composed of a repeating structure of the structural unit (f0) and the structural unit (a8), the proportion of the structural unit (f0) based on a total amount (100 mol %) of all structural units constituting the polymer compound is preferably 40 to 95 mol %, more preferably 50 to 90 mol %, and still more preferably 60 to 80 mol %.

The proportion of the structural unit (a8) in the polymer compound based on a total amount (100 mol %) of all structural units constituting the polymer compound is preferably 5 to 60 mol %, more preferably 10 to 50 mol %, and still more preferably 20 to 40 mol %.

Specific examples of fluorine resin components (F0) are shown below, but the present invention is not limited thereto.

In the following formulae, R^{α} is a hydrogen atom, a methyl group or a trifluoromethyl group.

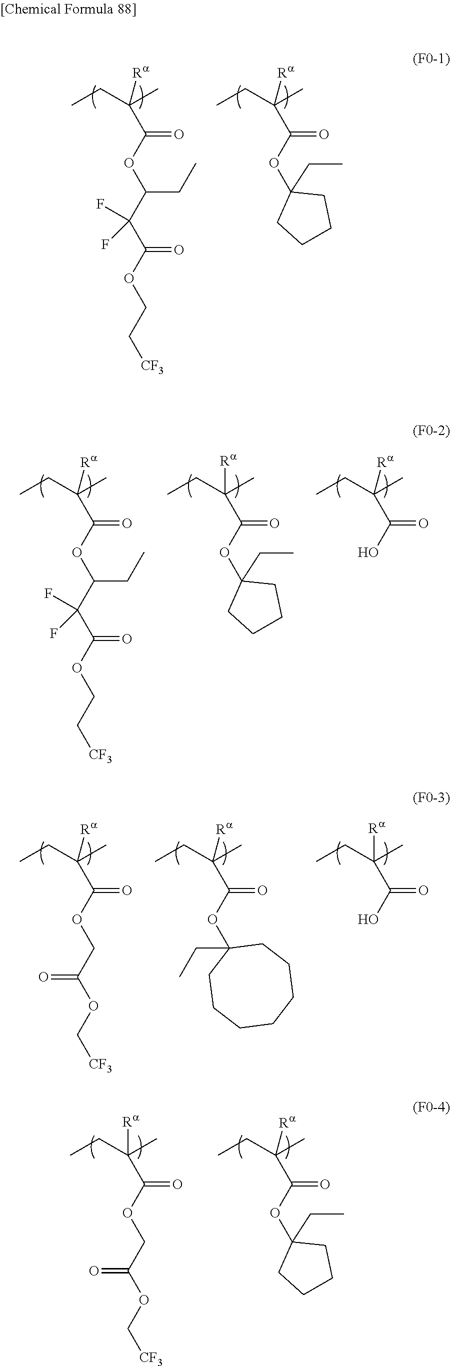

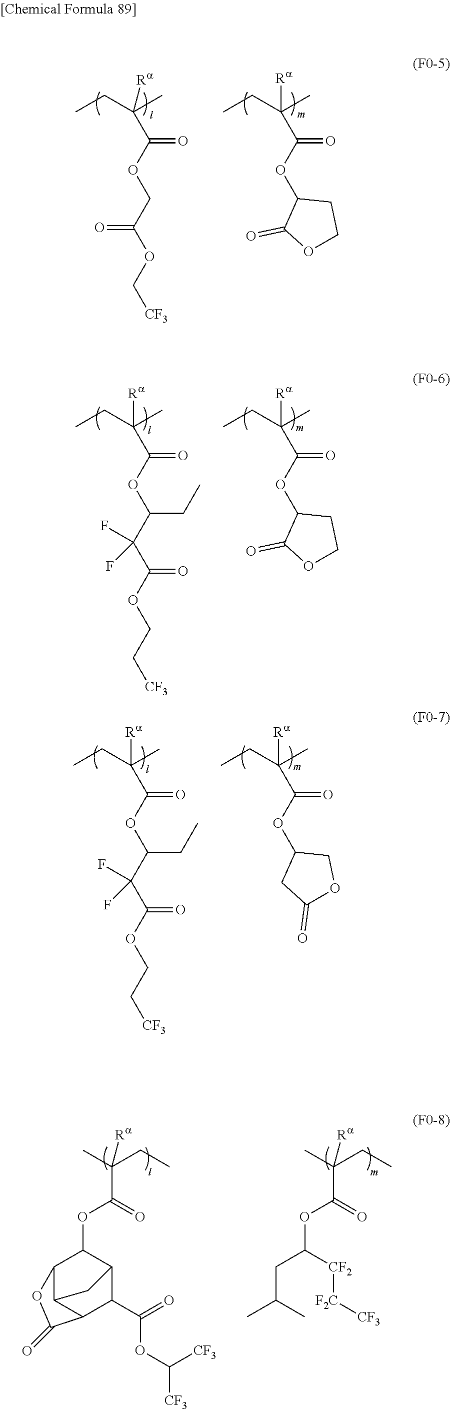

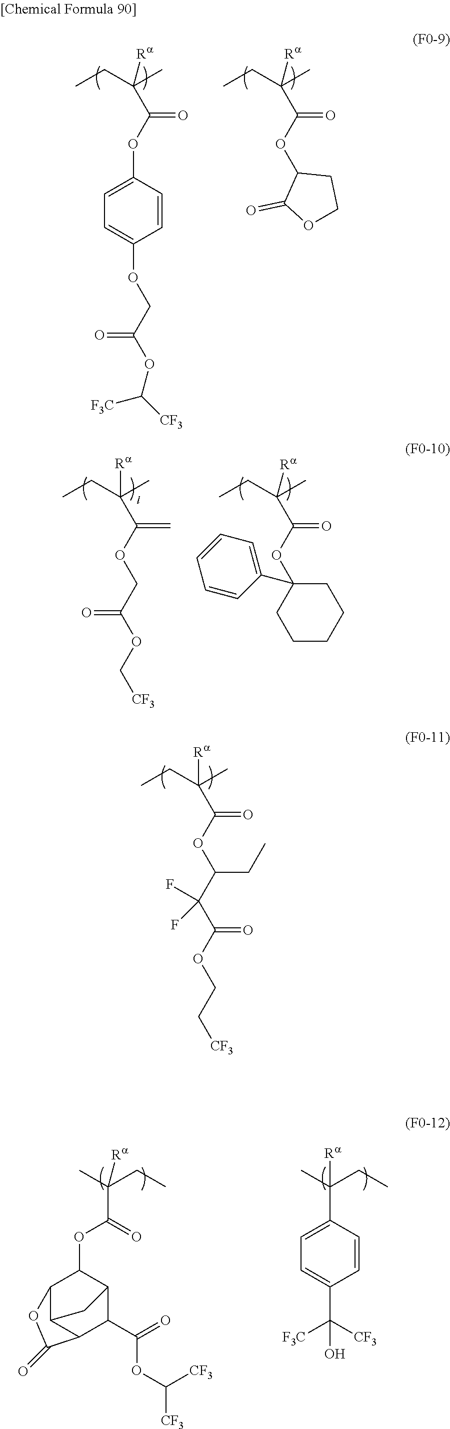

The weight average molecular weight (Mw) (in terms of polystyrene determined through gel permeation chromatography) of the component (F0) is preferably 1,000 to 50,000, more preferably 5,000 to 40,000, and most preferably 10,000 to 30,000. When the weight average molecular weight is set to equal to or smaller than the upper limit value of the range, the component has sufficient solubility in a resist solvent for use as a resist, and when the weight average molecular weight is set to equal to or larger than the lower limit value of the range, the resist film has favorable water repellency.

The dispersity (Mw/Mn) of the component (F0) is preferably 1.0 to 5.0, more preferably 1.0 to 3.0, and most preferably 1.0 to 2.5.

In the resist composition of the present embodiment, the components (F0) may be used alone or two or more thereof may be used in combination.

In the resist composition of the present embodiment, the content of the component (F0) with respect to 100 parts by mass of the component (A1) is preferably 0.5 to 15 parts by mass, more preferably 1 to 10 parts by mass, and still more preferably 3 to 7 parts by mass.

When the content of the component (F0) is set to be equal to or larger than the preferable lower limit value, lithography properties such as resolution and CDU are easily improved.

When the content of the component (F0) is set to be equal to or smaller than the preferable upper limit value, sensitivity is easily improved.

The resist composition of the present embodiment may or may not contain, as the component (F), in addition to the component (F0), a fluorine resin component (F₁) (hereinafter referred to as a “component (F₁)”) that does not correspond to the component (F0). The component (F) is preferably composed of only the component (F0).

The resist composition of one embodiment excludes a resist composition containing the component (F₁).

The proportion of the component (F0) in the component (F) based on a total mass of the component (F) is preferably 25 mass % or more, more preferably 50 mass % or more, still more preferably 75 mass % or more, and particularly preferably 100 mass %. When the proportion is 25 mass % or more, it is easier to form a resist pattern having various excellent lithography properties such as high sensitivity, improved resolution, and improved CDU.

### <Other Components>

The resist composition of the present embodiment may further contain other components in addition to the above component (A), component (B), and component (F). Examples of other components include the following component (D), component (E), and component (S).

### <<Base Component (D)>>

The resist composition of the present embodiment may contain, in addition to the component (A), a base component (component (D)) that traps (that is, controls diffusion of an acid) an acid generated upon exposure. The component (D) functions as a quencher (acid diffusion control agent) that traps an acid generated in the resist composition upon exposure.

Examples of components (D) include a photodecomposable base (D1) (hereinafter referred to as a “component (D1)”) that decomposes upon exposure and loses its ability to control acid diffusion, and a nitrogen-containing organic compound (D2) (hereinafter referred to as a “component (D2)”) that does not correspond to the component (Dl). Among these, the photodecomposable base (component (D1)) is preferable because it is easier to improve all properties of high sensitivity, reduced roughness, and minimization of the occurrence of coating defects.

The component (D1) and the component (D2) may be contained in the form of a compound or in a form that is incorporated as the above structural unit (a6) into the component (A1) or a combination of both forms. The compound exemplified as the following component (D1) may be used as the above acid generator component (component (B)) depending on the combination with other compounds.

### Component (D1)

The component (D1) is not particularly limited as long as it is decomposed upon exposure and loses its ability to control acid diffusion, and is preferably one or more compounds selected from the group consisting of a compound represented by the following General Formula (d1-1) (hereinafter referred to as a “component (dl-1)”), , a compound represented by the following General Formula (dl-2) (hereinafter referred to as a “component (dl-2)”) and a compound represented by the following General Formula (d1-3) (hereinafter referred to as a “component (dl-3)”).

The components (dl-1) to (dl-3) do not function as a quencher in the exposed part of the resist film because they decompose and lose their ability to control acid diffusion (basicity), but function as a quencher in the unexposed part of the resist film.

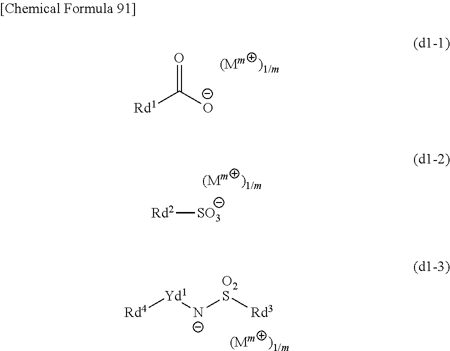

[in the formula, Rd¹ to Rd⁴ are a cyclic group which may have a substituent, a chain alkyl group which may have a substituent, or a chain alkenyl group which may have a substituent, here, in Rd² in Formula (dl-2), the carbon atom adjacent to the S atom has no fluorine atom bonded thereto, Yd¹ is a single bond or a divalent linking group, m is an integer of 1 or more, and M^{m+}'s are each independently an m-valent organic cation].
{Component (d1-1)}

### Anion Moiety

In Formula (d1-1), Rd¹ is a cyclic group which may have a substituent, a chain alkyl group which may have a substituent, or a chain alkenyl group which may have a substituent, and examples thereof include the same groups as for R′²⁰¹.

Among these, Rd¹ is preferably an aromatic hydrocarbon group which may have a substituent, an aliphatic cyclic group which may have a substituent, or a chain alkyl group which may have a substituent. Examples of substituents that these groups may have include a hydroxyl group, an oxo group, an alkyl group, an aryl group, a fluorine atom, a fluorinated alkyl group, the lactone-containing cyclic groups represented by General Formulae (a2-r-1) to (a2-r-7), an ether bond, an ester bond, and a combination thereof. When an ether bond or an ester bond is contained as a substituent, an intervening alkylene group may be provided, and the substituent in this case is preferably a linking group represented by each of Formulae (L-al-1) to (L-al-5). Here, when the aromatic hydrocarbon group, the aliphatic cyclic group, or the chain alkyl group for Rd¹ has, as a substituent, a linking group represented by each of General Formulae (L-al-1) to (L-al-8), in General Formulae (L-al-1) to (L-al-8), V′101 in General Formulae (L-al-1) to (L-al-8) bonds to a carbon atom constituting the aromatic hydrocarbon group, the aliphatic cyclic group, or the chain alkyl group for Rd¹ in Formula (dl-1).

Preferable examples of aromatic hydrocarbon groups include a phenyl group, a naphthyl group, and a polycyclic structure containing a bicyclooctane framework (a polycyclic structure composed of a bicyclooctane framework and another ring structure).

The aliphatic cyclic group is more preferably a group in which one or more hydrogen atoms have been removed from a polycycloalkane such as adamantane, norbornane, isobornane, tricyclo[5.2.1.0^{2,6}]decane, and tetracyclododecane.

The chain alkyl group preferably has 1 to 10 carbon atoms, and specific examples thereof include linear alkyl groups such as a methyl group, an ethyl group, a propyl group, a butyl group, a pentyl group, a hexyl group, a heptyl group, an octyl group, a nonyl group, and a decyl group; and branched alkyl groups such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

When the chain alkyl group is a fluorinated alkyl group having a fluorine atom or a fluorinated alkyl group as a substituent, the fluorinated alkyl group preferably has 1 to 11 carbon atoms, more preferably has 1 to 8 carbon atoms, and still more preferably 1 to 4 carbon atoms. The fluorinated alkyl group may contain an atom other than a fluorine atom. Examples of atoms other than a fluorine atom include an oxygen atom, a sulfur atom, and a nitrogen atom.

Preferable specific examples of anion moieties of the component (dl-1) are shown below.

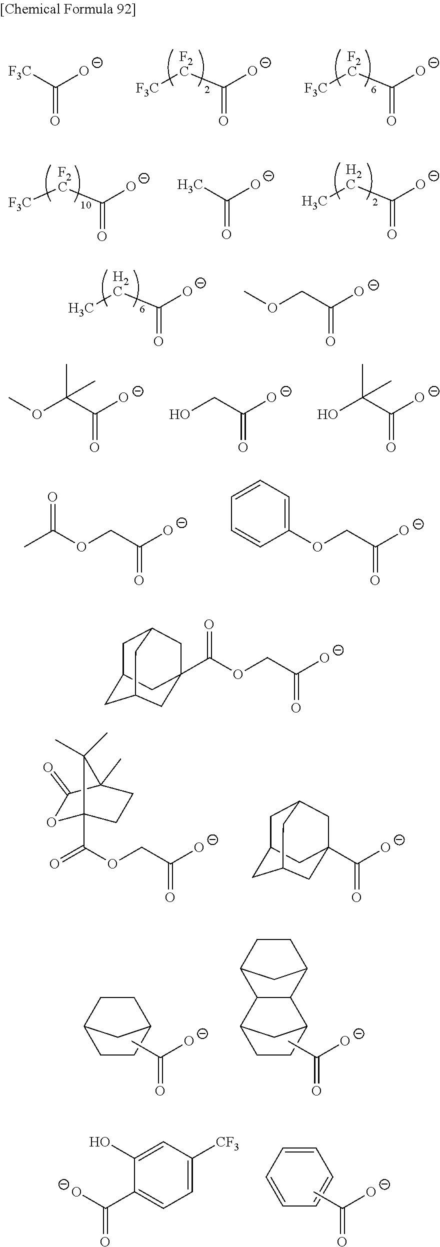

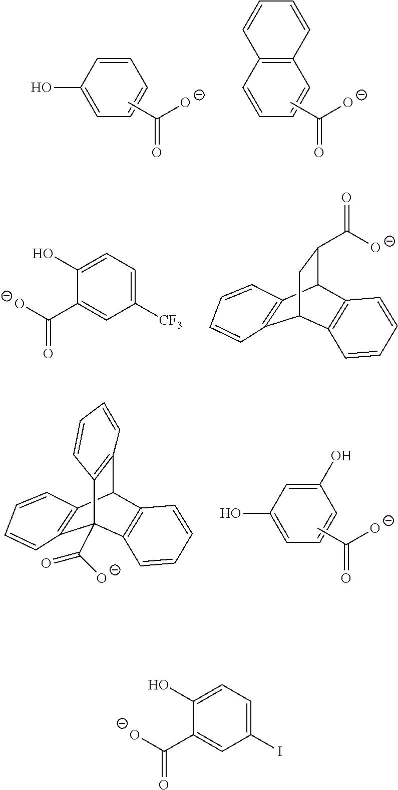

### Cation Moiety

In Formula (d1-1), M^{m+} is an m-valent organic cation.

Preferable examples of organic cations for M^{m+} include the same cations as those represented by General Formulae (ca-1) to (ca-3), and the cation represented by General Formula (ca-1) is more preferable, and the cations represented by Formulae (ca-1-1) to (ca-1-84) are still more preferable.

Components (d1-1) may be used alone or two or more thereof may be used in combination.

{Component (d1-2)}

### Anion Moiety

In Formula (dl-2), Rd² is a cyclic group which may have a substituent, a chain alkyl group which may have a substituent, or a chain alkenyl group which may have a substituent, and examples thereof include the same groups as for R′²⁰¹.

Here, in Rd², the carbon atom adjacent to the S atom has no fluorine atom bonded thereto (is not substituted with fluorine). Accordingly, the anion of the component (d1-2) becomes an appropriately weak acid anion, and the quenching ability of the component (D) is improved.

Rd² is preferably a chain alkyl group which may have a substituent or an aliphatic cyclic group which may have a substituent, and more preferably an aliphatic cyclic group which may have a substituent.

The chain alkyl group preferably has 1 to 10 carbon atoms and more preferably has 3 to 10 carbon atoms.

The aliphatic cyclic group is more preferably a group in which one or more hydrogen atoms have been removed from adamantane, norbornane, isobornane, tricyclo[5.2.1.0^{2,6}]decane, tetracyclododecane or the like (which may have a substituent); or a group in which one or more hydrogen atoms have been removed from camphor.

The hydrocarbon group for Rd² may have a substituent, and examples of substituents include the same substituents that the hydrocarbon group (aromatic hydrocarbon group, aliphatic cyclic group, chain alkyl group) for Rd¹ in Formula (dl-1) may have.

Preferable specific examples of anion moieties of the component (dl-2) are shown below.

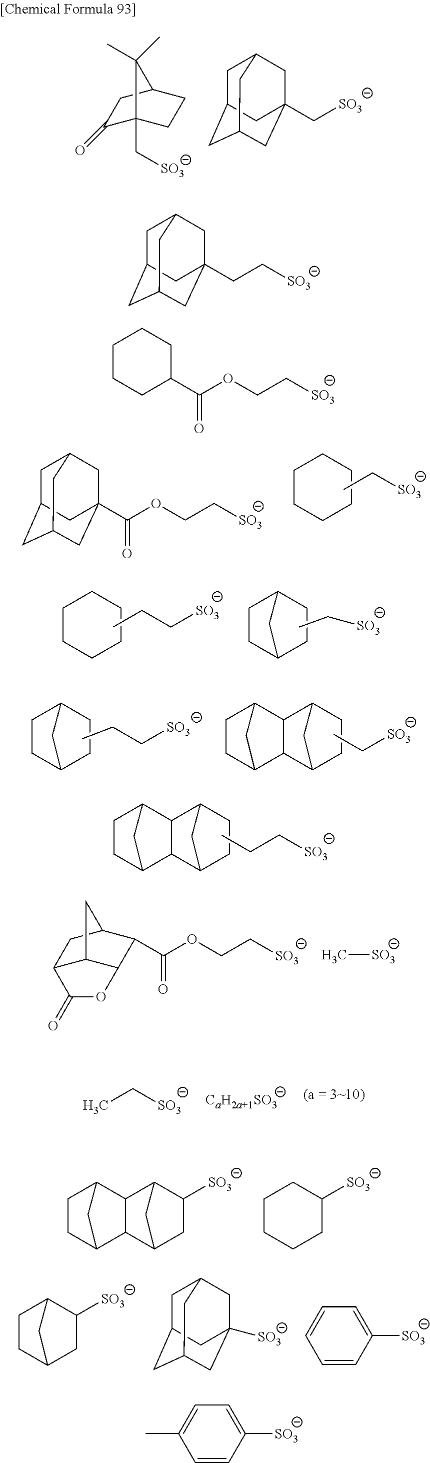

### Cation Moiety

In Formula (dl-2), M^{m+} is an m-valent organic cation, and is the same as M^{m+} in Formula (d1-1).

Components (d1-2) may be used alone or two or more thereof may be used in combination.

{Component (d1-3)}

### Anion Moiety

In Formula (dl-3), Rd³ is a cyclic group which may have a substituent, a chain alkyl group which may have a substituent, or a chain alkenyl group which may have a substituent, and examples thereof include the same as those for R′²⁰¹, and Rd³ is preferably a fluorine atom-containing cyclic group, a chain alkyl group, or a chain alkenyl group. Among these, a fluorinated alkyl group is preferable, and the same fluorinated alkyl group as for Rd¹ is more preferable.

In Formula (d1-3), Rd⁴ is a cyclic group which may have a substituent, a chain alkyl group which may have a substituent, or a chain alkenyl group which may have a substituent, and examples thereof include the same as those for R′²⁰¹.

Among these, an alkyl group, an alkoxy group, an alkenyl group, or a cyclic group which may have a substituent is preferable.

The alkyl group for Rd⁴ is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. Some hydrogen atoms in the alkyl group for Rd⁴ may be substituted with a hydroxy group, a cyano group or the like.

The alkoxy group for Rd⁴ is preferably an alkoxy group having 1 to 5 carbon atoms, and specific examples of alkoxy groups having 1 to 5 carbon atoms include a methoxy group, an ethoxy group, an n-propoxy group, an iso-propoxy group, an n-butoxy group, and a tert-butoxy group. Among these, a methoxy group or an ethoxy group is preferable.

Examples of alkenyl groups for Rd⁴ include the same alkenyl groups as for R′²⁰¹, and a vinyl group, a propenyl group (allyl group), a 1-methylpropenyl group, and a 2-methylpropenyl group are preferable. These groups may further contain, as a substituent, an alkyl group having 1 to 5 carbon atoms or a halogenated alkyl group having 1 to 5 carbon atoms.

Examples of cyclic groups for Rd⁴ include the same cyclic groups as for R′²⁰¹, and an alicyclic group in which one or more hydrogen atoms have been removed from a cycloalkane such as cyclopentane, cyclohexane, adamantane, norbornane, isobornane, tricyclo[5.2.1.0^{2,6}]decane, or tetracyclododecane, or an aromatic group such as a phenyl group and a naphthyl group is preferable. When Rd⁴ is an alicyclic group, the resist composition is easily dissolved in an organic solvent and thus exhibits favorable lithography properties. In addition, when Rd⁴ is an aromatic group, in lithography using EUV or the like as an exposure light source, the resist composition exhibits excellent light absorption efficiency and exhibits favorable sensitivity and lithography properties.

In Formula (d1-3), Yd¹ is a single bond or a divalent linking group.

The divalent linking group for Yd¹ is not particularly limited, and examples thereof include a divalent hydrocarbon group which may have a substituent (an aliphatic hydrocarbon group, and an aromatic hydrocarbon group), and a heteroatom-containing divalent linking group. Examples of these groups each include the same divalent hydrocarbon group which may have a substituent and heteroatom-containing divalent linking group exemplified in the description of the divalent linking group for Ya²¹ in Formula (a2-1).

Yd¹ is preferably a carbonyl group, an ester bond, an amide bond, an alkylene group or a combination thereof. The alkylene group is more preferably a linear or branched alkylene group and still more preferably a methylene group or an ethylene group.

Preferable specific examples of anion moieties of the component (d1-3) are shown below.

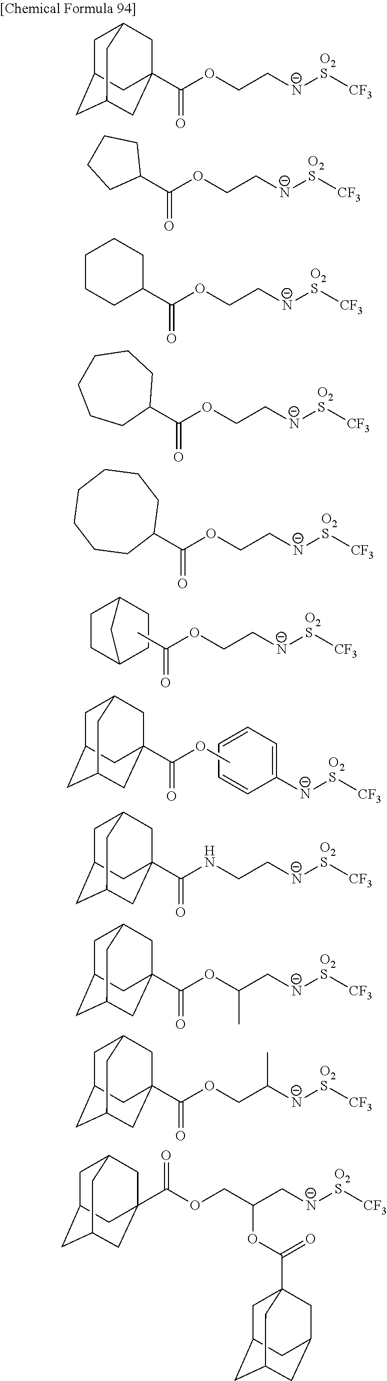

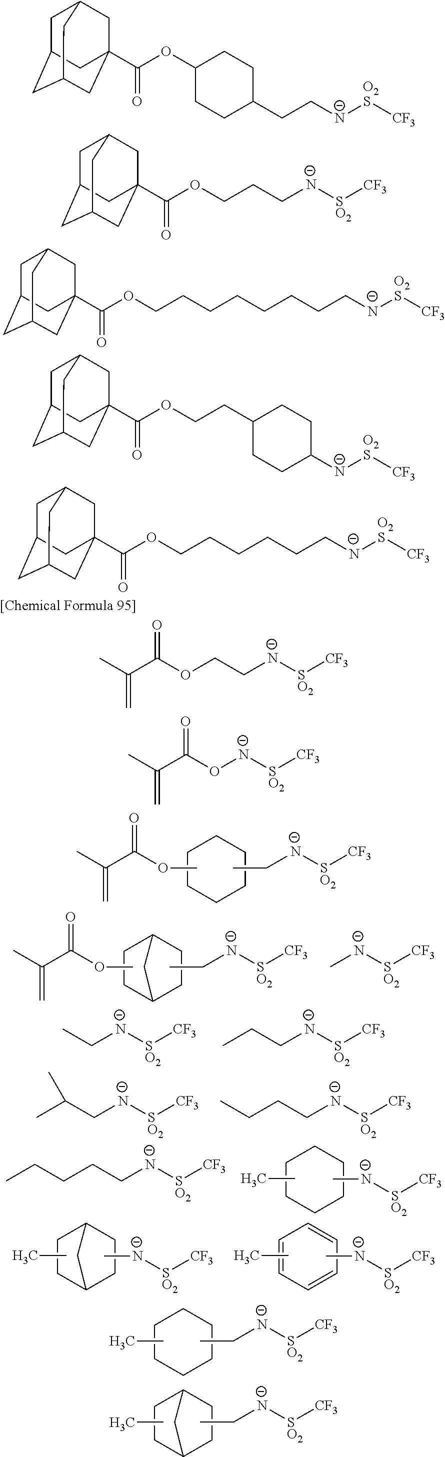

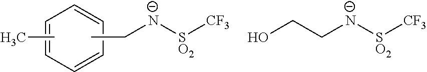

### Cation Moiety

In Formula (d1-3), M^{m+} is an m-valent organic cation, and is the same as M^{m+} in Formula (d1-1).

The components (d1-3) may be used alone or two or more thereof may be used in combination.

As the component (D1), any one of the components (d1-1) to (dl-3) may be used alone or two or more thereof may be used in combination.

When the resist composition contains the component (D1), the content of the component (D1) in the resist composition with respect to 100 parts by mass of the component (A) is preferably 0.5 to 15 parts by mass, more preferably 1 to 12 parts by mass, and still more preferably 2 to 10 parts by mass.

The component (D1) preferably includes the component (dl-1).

The content of the component (dl-1) in the entire component (D1) is preferably 50 mass % or more, more preferably 70 mass % or more, and still more preferably 90 mass % or more, and the component (D1) may be composed of only the compound component (d1-1).

### Method for Producing Component (D1):

The method for producing the component (dl-1) and component (d1-2) is not particularly limited, and they can be produced by known methods.

In addition, the method of producing the component (dl-3) is not particularly limited, and the component can be produced by, for example, the same method described in US2012-0149916.

Although the compound of the component (Dl) has been shown as an example of a base component (component (D)) that traps an acid generated upon exposure, the compound of the component (D1) may also be used as the component (B).

For example, in the resist composition of the present embodiment, the compound of the component (D1) may be used as the component (B), and a compound that generates an acid with a lower acidity than the acid generated by the compound of the component (D1) upon exposure may be used as the component (D). In addition, in the resist composition of the present embodiment, the compound of the component (D1) may be used as the component (B), and the following component (D2) may be used as the component (D).

### Component (D2)

The component (D) may contain a nitrogen-containing organic compound component (hereinafter referred to as a “component (D2)”) that does not correspond to the component (D1).

The component (D2) is not particularly limited as long as it functions as an acid diffusion control agent and does not correspond to the component (Dl), and any known component may be optionally used. Among these, an aliphatic amine is preferable, and among these, particularly, a secondary aliphatic amine or a tertiary aliphatic amine is more preferable.

The aliphatic amine is an amine having one or more aliphatic groups, and the aliphatic group preferably has 1 to 12 carbon atoms.

Examples of aliphatic amines include amines in which at least one hydrogen atom of ammonia NH₃ is substituted with an alkyl group or hydroxyalkyl group having 12 or fewer carbon atoms (alkylamines or alkyl alcohol amines) and cyclic amines.

Specific examples of alkylamines and alkyl alcohol amines include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among these, a trialkylamine having 6 to 30 carbon atoms is more preferable, and tri-n-pentylamine or tri-n-octylamine is particularly preferable.

Examples of cyclic amines include a heterocyclic compound containing a nitrogen atom as a heteroatom. The heterocyclic compound may be a monocyclic compound (aliphatic monocyclic amine) or a polycyclic compound (aliphatic polycyclic amine).

Specific examples of aliphatic monocyclic amines include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris {2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate, and triethanolamine triacetate is preferable.

In addition, an aromatic amine may be used as the component (D2).

Examples of aromatic amines include 4-dimethylaminopyridine, pyrrole, indole, pyrazole, imidazole, derivatives thereof, tribenzylamine, 2,6-diisopropylaniline, N-tert-butoxycarbonylpyrrolidine, 2,6-di-tert-butylpyridine, and 2,6-di-tert-butylpyridine.

Components (D2) may be used alone or two or more thereof may be used in combination.

When the resist composition contains the component (D2), the content of the component (D2) in the resist composition with respect to 100 parts by mass of the component (A) is generally used in a range of 0.01 to 5 parts by mass. Within the above range, a resist pattern shape, post-exposure temporal stability and the like are improved.

<<at Least One Compound (E) Selected from the Group Consisting of Organic Carboxylic Acid, Phosphorus Oxoacid and Derivatives Thereof>>

In order to prevent deterioration of sensitivity and improve a resist pattern shape and post-exposure temporal stability, the resist composition of the present embodiment can contain, as an optional component, at least one compound (E) selected from the group consisting of an organic carboxylic acid, a phosphorus oxoacid and derivatives thereof (hereinafter referred to as a “component (E)”).

Specific examples of organic carboxylic acids include acetic acid, malonic acid, citric acid, malic acid, succinic acid, benzoic acid, and salicylic acid, and among these, salicylic acid is preferable.

Examples of phosphorus oxoacids include phosphoric acid, phosphonic acid, and phosphinic acid, and among these, phosphonic acid is particularly preferable.

In the resist composition of the present embodiment, the components (E) may be used alone or two or more thereof may be used in combination.

When the resist composition contains the component (E), the content of the component (E) with respect to 100 parts by mass of the component (A) is preferably 0.01 to 5 parts by mass, and more preferably 0.05 to 3 parts by mass. Within the above range, lithography properties are further improved.

### <<Organic Solvent Component (S)>>

The resist composition of the present embodiment can be produced by dissolving a resist material in an organic solvent component (hereinafter referred to as a “component (S)”).

In the resist composition of the present embodiment, the components (S) may be used alone or a mixed solvent of two or more thereof may be used. Among these, propylene glycol monomethyl ether acetate (PGMEA), propylene glycol monomethyl ether (PGME), γ-butyrolactone, ethyl lactate (EL), and cyclohexanone are preferable.

In addition, the component (S) is preferably a mixed solvent in which PGMEA and a polar solvent are mixed. The mixing ratio (mass ratio) may be appropriately determined in consideration of the compatibility between PGMEA and the polar solvent.

As the component (S), a mixed solvent of at least one selected from among PGMEA and EL and γ-butyrolactone is also preferable. In this case, the mixing ratio (mass ratio) between the former and the latter is preferably 70:30 to 95:5.

The amount of the component (S) used is not particularly limited and is appropriately set according to the coating film thickness at a concentration at which it can be applied to a substrate and the like. Generally, the component (S) is used so that the solid content concentration of the resist composition is within a range of 0.1 to 20 mass %, and preferably 0.2 to 15 mass %.

In the resist composition of the present embodiment, the resist material may be dissolved in the component (S), and impurities and the like may be then removed using the polyimide porous film, the polyamide-imide porous film or the like. For example, the resist composition may be filtered using a filter made of a polyimide porous film, a filter made of a polyamide-imide porous film, a filter made of a polyimide porous film and a polyamide-imide porous film or the like. As the polyimide porous film and the polyamide-imide porous film, for example, those described in Japanese Unexamined Patent Application, First Publication No. 2016-155121 may be exemplified.

The resist composition of the present embodiment described above contains the compound (B0) and the fluorine resin component (F0) having the structural unit (f0). When the compound (B0) and the fluorine resin component (F0) are used in combination, it is possible to achieve a resist composition exhibiting high sensitivity, high resolution, and low CDU.

The reason why the resist composition of the present embodiment exhibits the above effects is not clear, but is inferred to be as follows.

Because the compound (B0) has an aromatic ring substituted with iodine, it exhibits high absorption efficiency for extreme ultraviolet (EUV) and electron beam (EB). On the other hand, the presence of iodine atoms tends to decrease the solubility in a development solution. However, since the compound (B0) has an acid-decomposable group (Rpg), the acid-dissociable group of the acid-decomposable group dissociates in the exposed part of the resist film. Thereby, the acid-decomposable group is converted into a polar group, and the solubility in a development solution is improved. That is, when the compound (B0) contains an aromatic ring substituted with an iodine atom and an acid-decomposable group, it becomes more soluble in a development solution in the exposed part of the resist film and less soluble in a development solution in the unexposed part of the resist film. In addition, the compound (B0) has a ring structure introduced between the acid-decomposable group and the iodine-substituted aromatic ring and/or between the iodine-substituted aromatic ring and the sulfonate group. Since this ring structure is a relatively bulky structure, the acid diffusion length is appropriately reduced.

The fluorine resin component (F0) has a structural unit (f0), and upon contact with an alkaline development solution, the base-dissociable group of the structural unit (f0) dissociates, thereby exhibiting solubility in the alkaline development solution. After alkaline development, hydrophilicity is improved due to the structural unit (f0) from which the base-dissociable group has dissociated. It is thought that, when this fluorine resin component (F0) is combined with the compound (B0), the uniformity of resist film dissolution in the alkaline development process and the subsequent water rinse process can be improved.

Due to these synergistic effects, the resist composition of the present embodiment is inferred to exhibit improved sensitivity and resolution and reduced CDU.

### (Resist Pattern Formation Method)

A resist pattern formation method according to a second aspect of the present invention is a method including a step of forming a resist film on a support using the resist composition according to the first aspect of the present invention, a step of exposing the resist film, and a step of developing the exposed resist film to form a resist pattern.

One embodiment of the resist pattern formation method is, for example, a resist pattern formation method performed as follows.

First, the resist composition of the above embodiment is applied onto a support using a spinner or the like and subjected to a bake (post apply bake (PAB)) treatment, for example, under a temperature condition of 80 to 150° C. for 40 to 120 seconds, preferably 60 to 90 seconds, to form a resist film.

Next, the resist film is subjected to exposure using, for example, an exposure device such as an electron beam lithography device or an ArF exposure device, by exposure through a mask (mask pattern) on which a predetermined pattern is formed, or by selective exposure through lithography using direct electron beam emission without using a mask pattern, and then subjected to a bake (post exposure bake (PEB)) treatment, for example, under a temperature condition of 80 to 150° C. for 40 to 120 seconds, preferably for 60 to 90 seconds.

Next, the resist film is subjected to a development treatment. The development treatment is performed using an alkaline development solution in the alkaline development process, and using a development solution containing an organic solvent (organic development solution) in the solvent development process.

After the development treatment, a rinse treatment is preferably performed. In the alkaline development process, the rinse treatment is preferably a water rinse using pure water, and in the solvent development process, a rinse solution containing an organic solvent is preferably used.

In the solvent development process, after the development treatment or the rinse treatment, a treatment of removing the development solution or rinse solution adhered to the pattern using a supercritical fluid may be performed.

After the development treatment or after the rinse treatment, drying is performed. In addition, in some cases, a baking treatment (post-baking) may be performed after the development treatment.

The support is not particularly limited, and any conventionally known support can be used, and examples thereof include a substrate for an electronic component and a substrate on which a predetermined wiring pattern is formed. More specific examples thereof include a silicon wafer, a substrate made of a metal such as copper, chromium, iron, or aluminum, and a glass substrate. As the material of the wiring pattern, for example, copper, aluminum, nickel, gold or the like can be used.

The wavelength used for exposure is not particularly limited, and radiation such as ArF excimer laser, KrF excimer laser, F₂ excimer laser, extreme ultraviolet (EUV) radiation, vacuum ultraviolet (VUV), electron beam (EB), X-rays, and soft X-rays can be used.

The resist pattern formation method of the present embodiment is particularly useful for a method in which, in the step of exposing the resist film, the resist film is exposed to extreme ultraviolet (EUV) or electron beams (EB).

The resist film exposure method may be general exposure (dry exposure) that is performed in air or in an inert gas such as nitrogen, or may be liquid immersion exposure (liquid immersion lithography).

The liquid immersion exposure is an exposure method in which the space between the resist film and the lowest lens of the exposure device is filled in advance with a solvent (liquid immersion medium) that has a larger refractive index than air, and exposure (immersion exposure) is performed in this state.

The liquid immersion medium is preferably a solvent having a larger refractive index than air and having a smaller refractive index than the resist film to be exposed, and examples thereof include water, a fluorine-based inert liquid, a silicone solvent, and a hydrocarbon solvent.

As the liquid immersion medium, water is preferably used.

Examples of alkaline development solutions used in the development treatment in the alkaline development process include a 0.1 to 10 mass % tetramethylaimnonium hydroxide (TMAH) aqueous solution.

The organic solvent contained in the organic development solution used in the development treatment in the solvent development process may be any solvent that can dissolve the component (A) (the component (A) before exposure), and can be appropriately selected from among known organic solvents. Specific examples thereof include polar solvents such as a ketone solvent, an ester solvent, an alcohol solvent, a nitrile solvent, an amide solvent, and an ether solvent, and a hydrocarbon solvent.

Examples of ester solvents include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl 3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of nitrile solvents include acetonitrile, propionitrile, valeronitrile, and butyronitrile.

As necessary, known additives can be added to the organic development solution. Examples of additives include a surfactant. The surfactant is not particularly limited, and for example, ionic or non-ionic fluorine-based and/or silicone-based surfactants can be used.

The development treatment can be performed by a known development method, for example, a method of immersing a support in a development solution for a certain time (dip method), a method of raising a development solution on the surface of a support by surface tension and leaving it for a certain time (puddle method), a method of spraying a development solution to the surface of a support (spray method), or a method of continuously applying a development solution by scanning a development solution application nozzle at a certain speed onto a support that rotates at a certain speed (dynamic dispensing method).

As the organic solvent contained in the rinse solution used in the rinse treatment after the development treatment in the solvent development process, for example, one that does not easily dissolve the resist pattern that is appropriately selected from among the organic solvents exemplified as the organic solvents used in the organic development solution can be used. Generally, at least one solvent selected from among a hydrocarbon solvent, a ketone solvent, an ester solvent, an alcohol solvent, an amide solvent and an ether solvent is used.

These organic solvents may be used alone or two or more thereof may be used in combination. In addition, they may be used in combination with other organic solvents or water.

The rinse treatment (washing treatment) using a rinse solution is performed by a known rinse method. Examples of rinse treatment methods include a method of continuously applying a rinse solution onto a support that rotates at a certain speed (rotary application method), a method of immersing a support in a rinse solution for a certain time (dip method), and a method of spraying a rinse solution to the surface of a support (spray method).

According to the resist pattern formation method of the present embodiment described above, since the above resist composition is used, it is possible to form a resist pattern with a favorable shape in which favorable sensitivity is maintained during resist pattern formation, and lithography properties such as improved resolution and reduced CDU of the resist pattern are further improved.

It is preferable that the resist composition of the above embodiment and various materials (for example, a resist solvent, a development solution, a rinse solution, a composition for forming an anti-reflective film, and a composition for forming a top coat) used in the pattern formation method of the above embodiment do not contain impurities such as metals, halogen-containing metal salts, acids, alkalis, and components containing sulfur atoms or phosphorus atoms. Here, examples of metal atom-containing impurities include Na, K, Ca, Fe, Cu, Mn, Mg, Al, Cr, Ni, Zn, Ag, Sn, Pb, Li, and salts thereof. The content of impurities contained in these materials is preferably 200 ppb or less, more preferably 1 ppb or less, still more preferably 100 ppt (parts per trillion) or less, particularly preferably 10 ppt or less, and most preferably substantially zero (equal to or smaller than the detection limit of the measurement device).

### EXAMPLES

The present invention will be described in more detail below with reference to examples, but the present invention is not limited to these examples.

### Preparation of Resist Composition

### Examples 1 to 19 and Comparative Examples 1 to 7

The components shown in Tables 1 and 2 were mixed and dissolved to prepare resist compositions of examples.

TABLE 1

| | Component (A) | Component (B) | Component (D) | Component (F) | Component (S) |
|---|---|---|---|---|---|
| Example 1 | (A1)-1 [100] | (B0)-8 [15] | (D1)-1 [5] | (F0)-1 [5] | (S)-1 [6000] |
| Example 2 | (A1)-1 [100] | (B0)-8 [15] | (D1)-1 [5] | (F0)-2 [5] | (S)-1 [6000] |
| Example 3 | (A1)-1 [100] | (B0)-8 [15] | (D1)-1 [5] | (F0)-3 [5] | (S)-1 [6000] |
| Example 4 | (A1)-1 [100] | (B0)-8 [15] | (D1)-1 [5] | (F0)-4 [5] | (S)-1 [6000] |
| Example 5 | (A1)-1 [100] | (B0)-8 [15] | (D1)-1 [5] | (F0)-5 [5] | (S)-1 [6000] |
| Example 6 | (A1)-1 [100] | (B0)-8 [15] | (D1)-1 [5] | (F0)-6 [5] | (S)-1 [6000] |
| Example 7 | (A1)-1 [100] | (B0)-8 [15] | (D1)-1 [5] | (F0)-7 [5] | (S)-1 [6000] |
| Example 8 | (A1)-1 [100] | (B0)-8 [15] | (D1)-1 [5] | (F0)-8 [5] | (S)-1 [6000] |
| Example 9 | (A1)-1 [100] | (B0)-8 [15] | (D1)-1 [5] | (F0)-9 [5] | (S)-1 [6000] |
| Example 10 | (A1)-1 [100] | (B0)-8 [15] | (D1)-1 [5] | (F0)-10 [5] | (S)-1 [6000] |
| Example 11 | (A1)-1 [100] | (B0)-8 [15] | (D1)-1 [5] | (F0)-11 [5] | (S)-1 [6000] |
| Example 12 | (A1)-1 [100] | (B0)-8 [15] | (D1)-1 [5] | (F0)-12 [5] | (S)-1 [6000] |
| Example 13 | (A1)-1 [100] | (B0)-1 [15] | (D1)-1 [5] | (F0)-2 [5] | (S)-1 [6000] |
| Example 14 | (A1)-1 [100] | (B0)-2 [15] | (D1)-1 [5] | (F0)-2 [5] | (S)-1 [6000] |
| Example 15 | (A1)-1 [100] | (BO)-3 [15] | (D1)-1 [5] | (F0)-2 [5] | (S)-1 [6000] |
| Example 16 | (A1)-1 [100] | (B0)-4 [15] | (D1)-1 [5] | (F0)-2 [5] | (S)-1 [6000] |
| Example 17 | (A1)-1 [100] | (B0)-5 [15] | (D1)-1 [5] | (F0)-2 [5] | (S)-1 [6000] |
| Example 18 | (A1)-1 [100] | (B0)-6 [15] | (D1)-1 [5] | (F0)-2 [5] | (S)-1 [6000] |
| Example 19 | (A1)-1 [100] | (B0)-7 [15] | (D1)-1 [5] | (F0)-2 [5] | (S)-1 [6000] |

TABLE 2

| | Component (A) | Component (B) | Component (D) | Component (F) | Component (S) |
|---|---|---|---|---|---|
| Comparative Example 1 | (A1)-1 [100] | (B0)-1 [15] | (D1)-1 [5] | (F1)-1 [5] | (S)-1 [6000] |
| Comparative Example 2 | (A1)-1 [100] | (B1)-1 [15] | (D1)-1 [5] | (F0)-4 [5] | (S)-1 [6000] |
| Comparative Example 3 | (A1)-1 [100] | (B1)-2 [15] | (D1)-1 [5] | (F0)-8 [5] | (S)-1 [6000] |
| Comparative Example 4 | (A1)-1 [100] | (B1)-3 [15] | (D1)-1 [5] | (F0)-11 [5] | (S)-1 [6000] |
| Comparative Example 5 | (A1)-1 [100] | (B1)-1 [15] | (D1)-1 [5] | (F1)-1 [5] | (S)-1 [6000] |
| Comparative Example 6 | (A1)-1 [100] | (B1)-2 [15] | (D1)-1 [5] | (F1)-1 [5] | (S)-1 [6000] |
| Comparative Example 7 | (A1)-1 [100] | (B1)-3 [15] | (D1)-1 [5] | (F1)-1 [5] | (S)-1 [6000] |

The abbreviations in Tables 1 and 2 have the following meanings. The numbers in [ ] are the amounts added (parts by mass).
(A1)-1: polymer compound represented by the following Formula (A1-1). The weight average molecular weight (Mw) and the molecular weight dispersity (Mw/Mn) determined through GPC measurement (in terms of standard polystyrene) were 7,200 and 1.67, respectively. The copolymer composition ratio (ratio (molar ratio) of structural units in the structural formula) determined through ¹³C-NMR was l/m=50/50.

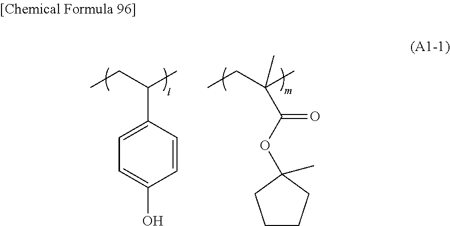

(B0)-1 to (B0)-8: acid generators containing the following compounds (B0-1) to (B0-8).

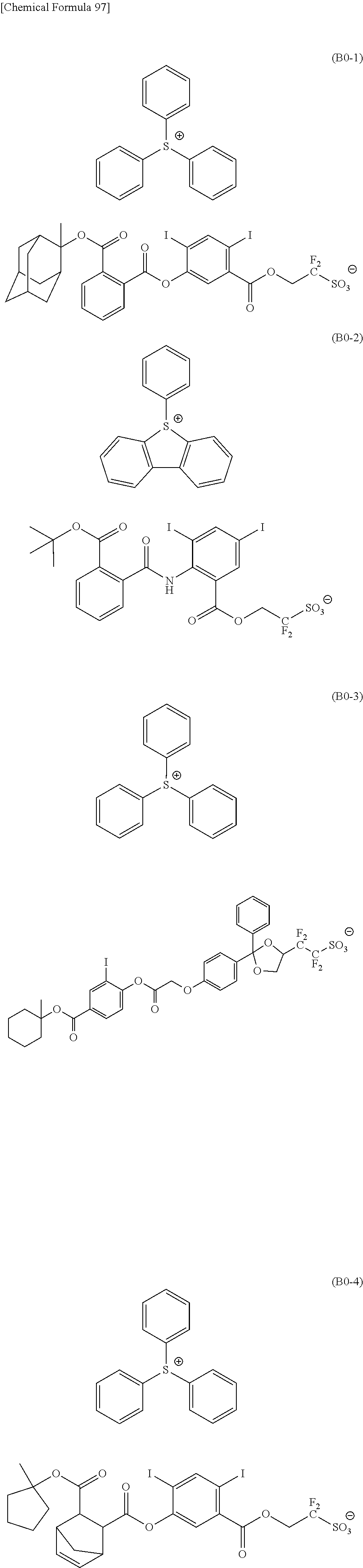

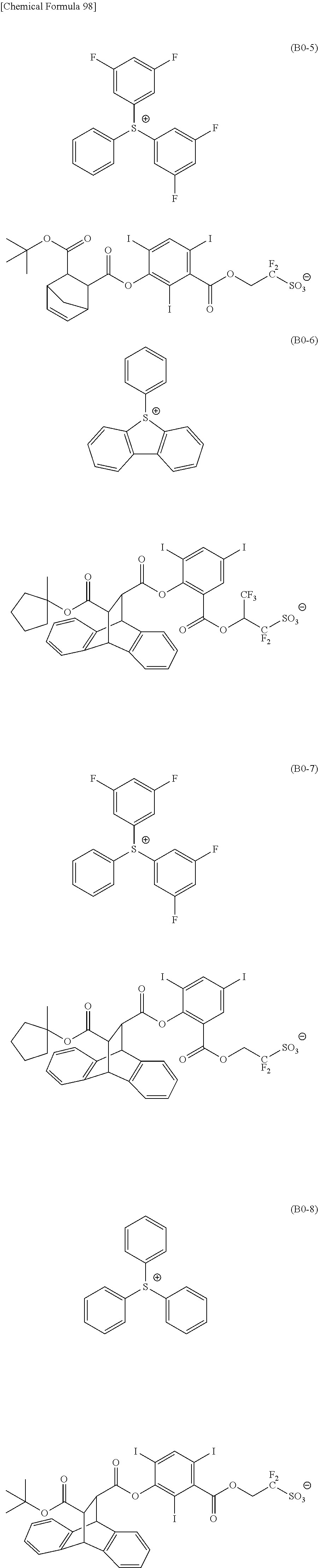

(B1)-1 to (B1)-3: acid generators containing the following compounds (B1-1) to (B1-3).

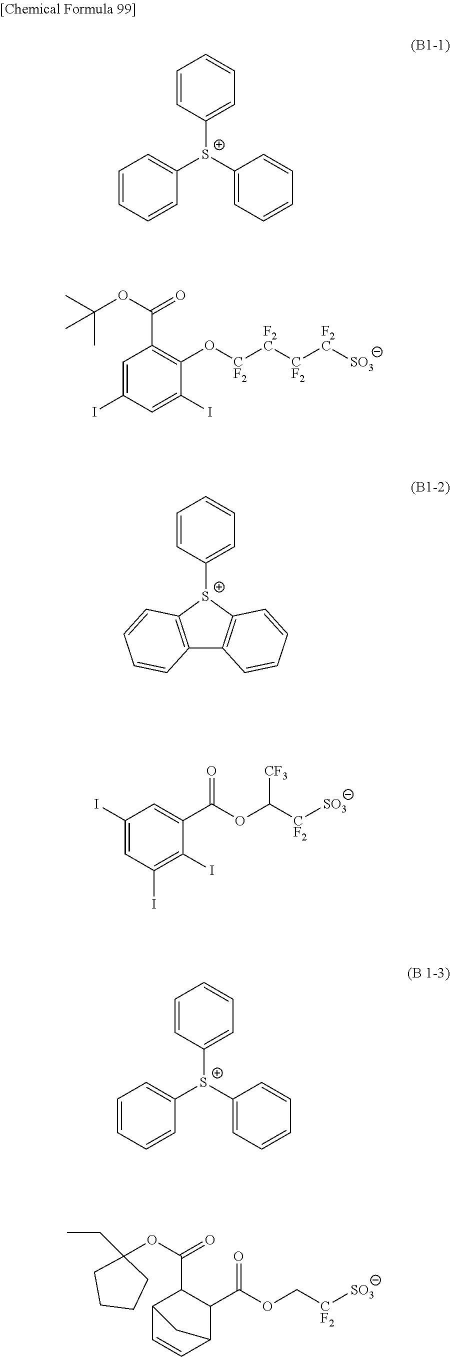

(D1)-1: acid diffusion control agent containing the following compound (D1-1).

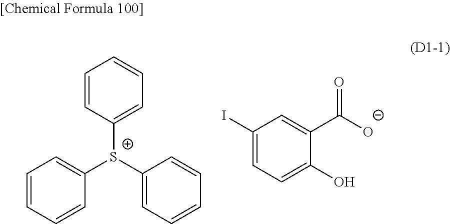 [SMILES: c1ccc([S+](c2ccccc2)c2ccccc2)cc1 ; O=C([O-])c1cc(I)ccc1O]

(F0)-1: fluorine resin component represented by the following Formula (F0-1). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-2: fluorine resin component represented by the following Formula (F0-2). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m/n=70/20/10.
(F0)-3: fluorine resin component represented by the following Formula (F0-3). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m/n=70/20/10.
(F0)-4: fluorine resin component represented by the following Formula (F0-4). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-5: fluorine resin component represented by the following Formula (F0-5). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-6: fluorine resin component represented by the following Formula (F0-6). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-7: fluorine resin component represented by the following Formula (F0-7). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-8: fluorine resin component represented by the following Formula (F0-8). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=50/50.
(F0)-9: fluorine resin component represented by the following Formula (F0-9). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-10: fluorine resin component represented by the following Formula (F0-10). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.
(F0)-11: fluorine resin component represented by the following Formula (F0-11). The weight average molecular weight (Mw) and the molecular weight dispersity (Mw/Mn) determined through GPC measurement (in terms of standard polystyrene) were 15,000, and 1.70, respectively.
(F0)-12: fluorine resin component represented by the following Formula (F0-12). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.

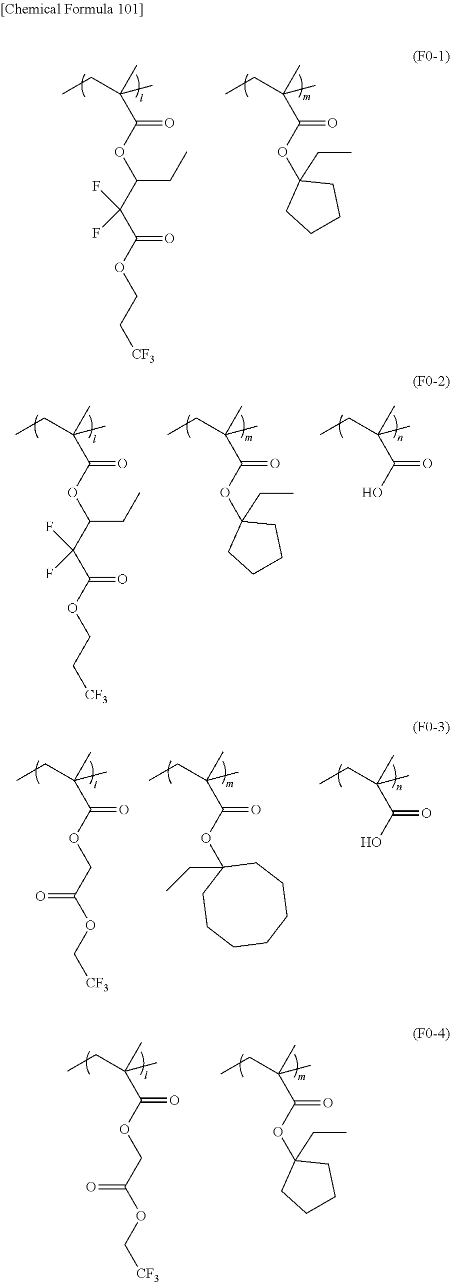

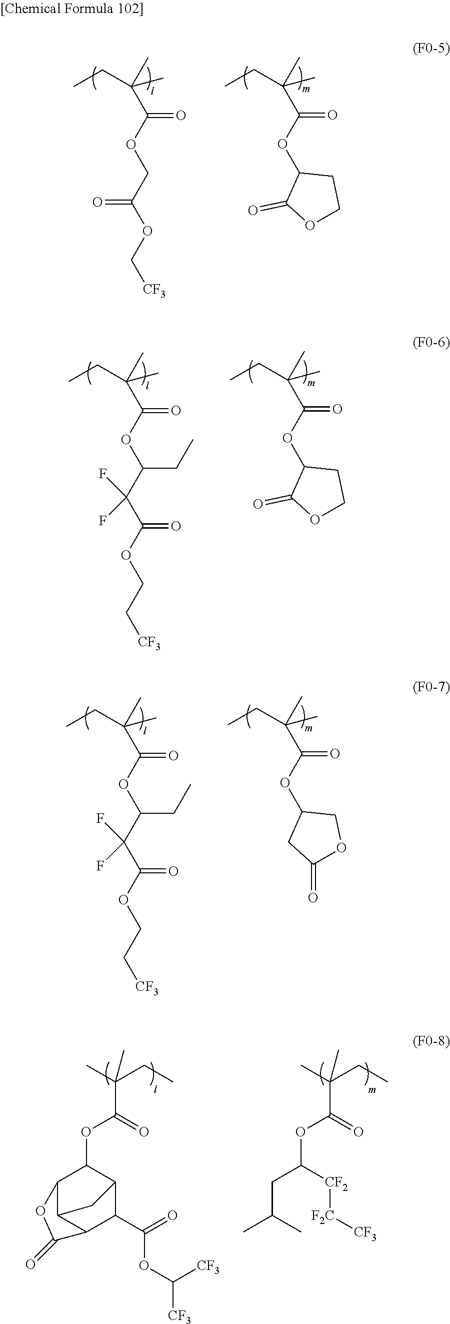

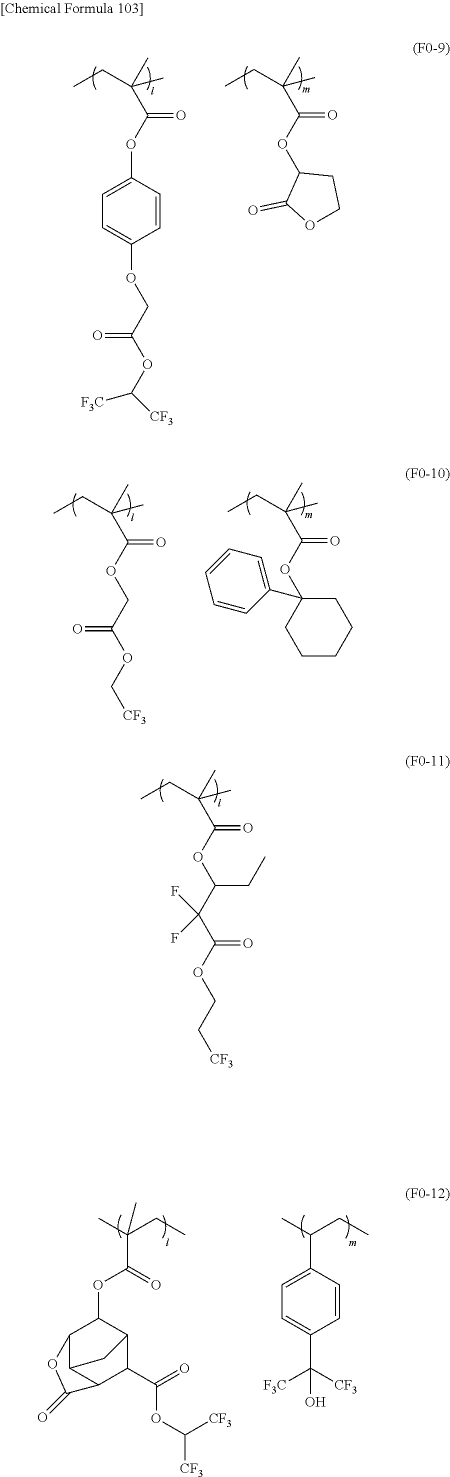

(F1)-1: fluorine resin component represented by the following Formula (F1-1). The weight average molecular weight (Mw) and the molecular weight dispersity (Mw/Mn) determined through GPC measurement (in terms of standard polystyrene) were 15,000, and 1.70, respectively. The copolymer composition ratio (ratio (molar ratio) of structural units in the structural formula) determined through ¹³C-NMR was l/m=70/30.

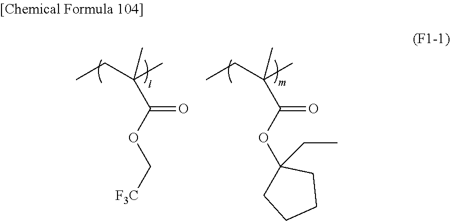

(S)-1: a mixed solvent of propylene glycol monomethyl ether acetate/propylene glycol monomethyl ether=60/40 (mass ratio).

### <Formation of Resist Pattern>

A resist composition of each example was applied onto a 12-inch silicon substrate on which a hexamethyldisilazane (HMDS) treatment was performed using a spinner, a pre-bake (PAB) treatment was performed on a hot plate at a temperature of 110° C. for 60 seconds, and drying was performed to form a resist film with a film thickness of 55 nm.

Next, the resist film was exposed to light using an EUV scanner NXE3400 (NXE3400 (NA0.33, inner c/outer σ=0.3/0.9, quadruple illumination) (commercially available from ASML) to form a contact hole pattern (hereinafter referred to as a “CH pattern”) in which holes with a diameter of 28 nm were arranged at equal intervals (a pitch of 56 nm).

Then, a post exposure bake (PEB) treatment was performed at 110° C. for 60 seconds.

Next, alkaline development was performed using a 2.38 mass % tetramethylammonium hydroxide (TMAH) aqueous solution “NMD-3” (product name, commercially available from Tokyo Ohka Kogyo Co., Ltd.) at 23° C. for 30 seconds.

Then, a water rinse treatment was performed using pure water for 15 seconds.

As a result, a CH pattern in which holes with a diameter of 28 nm were arranged at equal intervals (a pitch of 56 nm) was formed.

### [Evaluation of Sensitivity (Eop)]

The optimal exposure amount Eop (μC/cm²) at which a CH pattern with a target size was formed according to the above <Formation of resist pattern> was determined. The results are shown in Tables 3 and 4 as “Eop (nJ/cm²).”

### [Evaluation of Critical Dimension Uniformity (CDU) of Pattern Dimensions]

The CH pattern formed by the above <Formation of resist pattern> was observed from above the CH pattern using a critical dimension measurement SEM (scanning electron microscope, an acceleration voltage of 500 V, product name: CG6300, commercially available from Hitachi High-Tech Corporation), and the hole diameter (nm) of each hole was measured. Then, a value (3σ) three times the standard deviation (σ) calculated from the measurement result was determined. The results are shown in Tables 3 and 4 as “CDU (nm).”

A smaller 3σ determined in this manner indicates a higher dimension (CD) uniformity of the plurality of holes formed in the resist film.

### [Evaluation of Resolution]

When a CH pattern was formed by gradually decreasing the exposure amount from the optimal exposure amount Eop, the dimensions of the pattern that could be resolved without the residue were determined using a critical dimension measurement SEM (scanning electron microscope, acceleration voltage 500 V, product name: CG6300, commercially available from Hitachi High-Tech Corporation). The results are shown in Tables 3 and 4 as “limiting resolution (nm).”

TABLE 3

| | Eop (mJ/cm²) | CDU (nm) | Limiting resolution (nm) |
|---|---|---|---|
| Example 1 | 32 | 2.9 | 24 |
| Example 2 | 32 | 2.4 | 22 |
| Example 3 | 32 | 2.4 | 22 |
| Example 4 | 33 | 2.7 | 23 |
| Example 5 | 33 | 2.8 | 23 |
| Example 6 | 33 | 2.9 | 23 |
| Example 7 | 33 | 3.0 | 24 |
| Example 8 | 35 | 3.2 | 24 |
| Example 9 | 34 | 2.8 | 24 |
| Example 10 | 33 | 2.7 | 24 |
| Example 11 | 35 | 3.1 | 24 |
| Example 12 | 35 | 3.0 | 23 |
| Example 13 | 36 | 2.7 | 24 |
| Example 14 | 35 | 2.6 | 23 |
| Example 15 | 38 | 3.1 | 24 |
| Example 16 | 36 | 2.8 | 24 |
| Example 17 | 32 | 2.7 | 23 |
| Example 18 | 33 | 2.6 | 21 |
| Example 19 | 32 | 2.4 | 21 |

TABLE 4

| | Eop | CDU | Limiting |
|---|---|---|---|
| | (μC/cm²) | (nm) | resolution (nm) |
| Comparative Example 1 | 38 | 3.7 | 27 |
| Comparative Example 2 | 34 | 3.9 | 26 |
| Comparative Example 3 | 38 | 4.0 | 28 |
| Comparative Example 4 | 41 | 4.3 | 28 |
| Comparative Example 5 | 35 | 4.2 | 28 |
| Comparative Example 6 | 39 | 4.2 | 28 |
| Comparative Example 7 | 43 | 4.5 | 28 |

Based on the results shown in Tables 3 and 4, it was confirmed that the resist compositions of Examples 1 to 19 exhibited improvements in sensitivity, CDU, and limiting resolution compared to the resist compositions of Comparative Examples 1 to 7. Based on these results, it was confirmed that, when the component (B0) and the component (F0) were used in combination, lithography properties such as sensitivity, CDU, and limiting resolution were improved.

The present invention is not limited to the above descriptions, but it is only limited by the scope of the appended claims. Additions, omissions, substitutions and other modifications of the configuration can be made without departing from the scope of the present invention. The present invention is not limited to the above descriptions, but it is only limited by the scope of the appended claims.

What is claimed is:

1. A resist composition that generates an acid upon exposure and whose solubility in a development solution changes under the action of the acid, comprising:
a base material component (A) whose solubility in a development solution changes under the action of an acid;
an acid generator component (B) that generates an acid upon exposure; and
a fluorine additive component (F) that is decomposable in an alkaline development solution,
wherein the acid generator component (B) contains a compound (B0) represented by the following General Formula (bW), and
the fluorine additive component (F) contains a fluorine resin component (F0) having a structural unit (f0) represented by the following General Formula (f0): 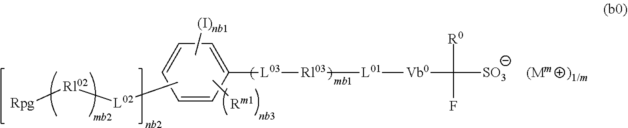
wherein Rpg is an acid-decomposable group, R1⁰¹ and R1⁰² are each independently a cyclic hydrocarbon group which may have a substituent, L⁰¹ is a divalent linking group or a single bond, L⁰² and L⁰³ are each independently a divalent linking group, R ^{m1} is a substituent other than an iodine atom, Vb⁰ is a single bond, an alkylene group or a fluorinated alkylene group, R⁰ is a hydrogen atom, a fluorinated alkyl group having 1 to 5 carbon atoms or a fluorine atom, nb1 is an integer of 1 to 4, nb2 is an integer of 1 to 4, nb3 is an integer of 0 to 3, and nb1+nb2+nb3≤5, mb1 and mb2 are each independently 0 or 1, and mb1 and mb2 are not both 0, M^{m+} is an m-valent organic cation, and m is an integer of 1 or more 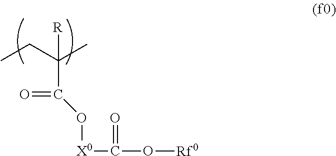
wherein R is a hydrogen atom, an alkyl group having 1 to 5 carbon atoms or a halogenated alkyl group having 1 to 5 carbon atoms, X⁰ is a divalent linking group having no acid-dissociable moiety, and Rf⁰ is an organic group having a fluorine atom.

2. The resist composition according to claim 1, wherein the compound (B0) is a compound represented by the following General Formula (b0-1) or (b0-2): 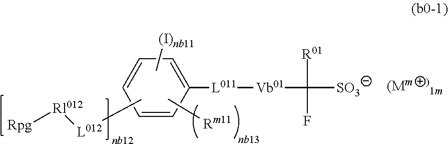
wherein Rpg is an acid-decomposable group, R1⁰¹² is a cyclic hydrocarbon group which may have a substituent, L⁰¹¹ is a divalent linking group or a single bond, L⁰¹² is a divalent linking group, R^{m11} is a substituent other than an iodine atom, Vb⁰¹ is a single bond, an alkylene group or a fluorinated alkylene group, R⁰¹ is a hydrogen atom, a fluorinated alkyl group having 1 to 5 carbon atoms or a fluorine atom, nb11 is an integer of 1 to 4, nb12 is an integer of 1 to 4, nb13 is an integer of 0 to 3, and nb11+nb12+nb13≤5, M^{m+} is an m-valent organic cation, and m is an integer of 1 or more, 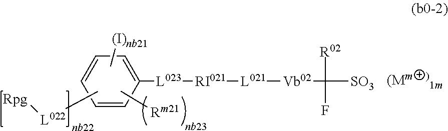
wherein Rpg is an acid-decomposable group, R1⁰²¹ is a cyclic hydrocarbon group which may have a substituent, L⁰²¹ is a divalent linking group or a single bond, L⁰²² and L⁰²³ are each independently a divalent linking group, Rⁿ¹²¹ is a substituent other than an iodine atom, Vb⁰² is a single bond, an alkylene group or a fluorinated alkylene group, R⁰² is a hydrogen atom, a fluorinated alkyl group having 1 to 5 carbon atoms or a fluorine atom, nb21 is an integer of 1 to 4, nb22 is an integer of 1 to 4, nb23 is an integer of 0 to 3, and nb21+nb22+nb23≤5, M^{m+} is an m-valent organic cation, and m is an integer of 1 or more.

3. The resist composition according to claim 1, wherein X0 in General Formula (f0) does not contain a cyclic group.

4. The resist composition according to claim 1, wherein the fluorine resin component (F0) further has a structural unit represented by the following General Formula (f1): 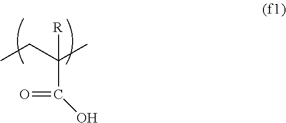
wherein R is a hydrogen atom, an alkyl group having 1 to 5 carbon atoms or a halogenated alkyl group having 1 to 5 carbon atoms.

5. The resist composition according to claim 1, wherein the fluorine resin component (F0) further has a structural unit having a lactone-containing cyclic group.

6. The resist composition according to claim 1, wherein the fluorine resin component (F0) further has a structural unit having an acid-dissociable group.

7. The resist composition according to claim 1, wherein the fluorine resin component (F0) further has a structural unit represented by the following General Formula (a8-1): 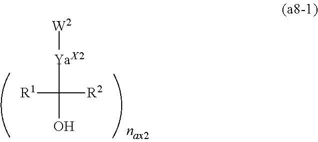
wherein W² is a polymerizable group-containing group, Ya^{x2} is a single bond or a (nₐₓ₂+1)-valent linking group, Ya^{x2} and W² may form a fused ring, R¹ is a fluorinated alkyl group having 1 to 12 carbon atoms, R² is an organic group having 1 to 12 carbon atoms, which may have a fluorine atom or a hydrogen atom, R² and Ya^{x2} may be bonded to each other to form a ring structure, and nₐₓ₂ is an integer of 1 to 3.

8. The resist composition according to claim 1, wherein X⁰ in General Formula (f0) is a linear aliphatic hydrocarbon group which may have a substituent or a branched aliphatic hydrocarbon group which may have a substituent.

9. A resist pattern formation method, comprising:
forming a resist film on a support using the resist composition according to claim 1 ;
exposing the resist film; and
developing the exposed resist film to form a resist pattern.
